SEQUENCE LISTING (152 sequences)

SEQ ID NO: 1 - protein, 6 aa, Artificial Sequence
  CDRL1
QSIGTS

SEQ ID NO: 2 - protein, 3 aa, Artificial Sequence
  CDRL2
YAS

SEQ ID NO: 3 - protein, 9 aa, Artificial Sequence
  CDRL3
QQSDSWPTT

SEQ ID NO: 4 - protein, 26 aa, Artificial Sequence
  FR1
DIVLTQTPAILSVSPGERVSFSCRAS

SEQ ID NO: 5 - protein, 17 aa, Artificial Sequence
  FR2
IHWYQQRTNGSPRLLIK

SEQ ID NO: 6 - protein, 36 aa, Artificial Sequence
  FR3
ESISGIPSRFSGSGSGTDFTLRINSVESEDLADYYC

SEQ ID NO: 7 - protein, 11 aa, Artificial Sequence
  FR4
FGGGTKLEIKR

SEQ ID NO: 8 - protein, 108 aa, Artificial Sequence
  Light chain variable (amino acid)
DIVLTQTPAILSVSPGERVSFSCRASQSIGTSIHWYQQRTNGSPRLLIKYASESISGIPSRFSGSGSGTDFTLRINSVESEDLADYYCQQSDSWPTTFGGGTKLEIKR

SEQ ID NO: 9 - DNA, 323 bp, Artificial Sequence
  Light chain variable (nucleotide sequence)
gacattgtgctgacccaaactccagccatcctgtctgtgagtccaggagaaagagtcagtttctcctgcagggccagtcagagcattggcacaagcatacactggtatcagcaaagaacaaatggttctccaaggcttctcataaaatatgcttctgagtctatctctgggatcccttccaggtttagtggcagtggatcagggacagattttactcttcgcatcaacagtgtggagtctgaggatcttgcagattattactgtcagcaaagtgatagctggccaaccacgttcggaggggggaccaagctggaaataaaacg

SEQ ID NO: 10 - protein, 8 aa, Artificial Sequence
  CDRH1
GYTFTSYY

SEQ ID NO: 11 - protein, 8 aa, Artificial Sequence
  CDRH2
IYPGNVNT

SEQ ID NO: 12 - protein, 14 aa, Artificial Sequence
  CDRH3
ARLGYYRYGGAMDY

SEQ ID NO: 13 - protein, 25 aa, Artificial Sequence
  FR1
EVQLQESGPELVKPGTSVRISCKAS

SEQ ID NO: 14 - protein, 17 aa, Artificial Sequence
  FR2
MHWVKQRPGQGLEWIGY

SEQ ID NO: 15 - protein, 38 aa, Artificial Sequence
  FR3
KYNEKFKGKATLTADKSSSTAYMQLSSLTSEDSAVYFC

SEQ ID NO: 16 - protein, 11 aa, Artificial Sequence
  FR4
WGQGTAVTVSS

SEQ ID NO: 17 - protein, 121 aa, Artificial Sequence
  Heavy chain variable (amino acid)
EVQLQESGPELVKPGTSVRISCKASGYTFTSYYMHWVKQRPGQGLEWIGYIYPGNVNTKYNEKFKGKATLTADKSSSTAYMQLSSLTSEDSAVYFCARLGYYRYGGAMDYWGQGTAVTVSS

SEQ ID NO: 18 - DNA, 363 bp, Artificial Sequence
  Heavy chain variable (nucleotide sequence)
gaggtacagctgcaggagtcaggacctgagctggtgaagcctgggacttcagtgaggatatcctgcaaggcttctggctacaccttcacaagctactatatgcattgggtgaagcagaggcctggacagggacttgagtggattggatacatttatcctggaaatgttaatactaaatacaatgagaagttcaagggcaaggccacactgactgcagacaaatcctccagcacagcctacatgcaactcagcagcctgacctctgaggactctgcggtctatttctgtgcaaggttgggctactataggtacgggggtgctatggactactggggtcaaggaaccgcagtcaccgtctcctca

SEQ ID NO: 19 - protein, 6 aa, Artificial Sequence
  CDRL1
QDISNY

SEQ ID NO: 20 - protein, 3 aa, Artificial Sequence
  CDRL2
YTS

SEQ ID NO: 21 - protein, 9 aa, Artificial Sequence
  CDRL3
QQYNKLPWT

SEQ ID NO: 22 - protein, 26 aa, Artificial Sequence
  FR1
DIVLTQTPSSLSASLGDRVTISCSAS

SEQ ID NO: 23 - protein, 17 aa, Artificial Sequence
  FR2
LNWYQQKPDGIVKLLIY

SEQ ID NO: 24 - protein, 36 aa, Artificial Sequence
  FR3
SLHSGVPSRFSGSGSGTDFSLTISNLEPEDIATYYC

SEQ ID NO: 25 - protein, 10 aa, Artificial Sequence
  FR4
FGGGTKLEIK

SEQ ID NO: 26 - protein, 107 aa, Artificial Sequence
  Light chain variable (amino acid)
DIVLTQTPSSLSASLGDRVTISCSASQDISNYLNWYQQKPDGIVKLLIYYTSSLHSGVPSRFSGSGSGTDFSLTISNLEPEDIATYYCQQYNKLPWTFGGGTKLEIK

SEQ ID NO: 27 - DNA, 322 bp, Artificial Sequence
  Light chain variable (nucleotide sequence)
gatattgtgctgacccagactccatcctccctgtctgcctctctgggagacagagtcaccatcagttgcagtgcaagtcaggacattagcaattatttaaactggtatcagcagaaaccagatggaattgttaaactcctgatctattacacatcaagtttacactcaggagtcccatcaaggttcagtggcagtgggtctgggacagatttttctctcaccatcagcaacctggaacctgaagatattgccacttactattgtcagcagtataataaacttccgtggacattcggtggaggcaccaagctggaaatcaaac

SEQ ID NO: 28 - protein, 9 aa, Artificial Sequence
  CDRH1
GYSITSGYY

SEQ ID NO: 29 - protein, 7 aa, Artificial Sequence
  CDRH2
MSYDGSN

SEQ ID NO: 30 - protein, 12 aa, Artificial Sequence
  CDRH3
SRALAYYGNFDY

SEQ ID NO: 31 - protein, 26 aa, Artificial Sequence
  FR1
LEVKLEESGPGLVKPSQSLSLTCSVT

SEQ ID NO: 32 - protein, 17 aa, Artificial Sequence
  FR2
WNWIRQFPGNKLEWMGF

SEQ ID NO: 33 - protein, 38 aa, Artificial Sequence
  FR3
NYNPSLKNRISITRDTSKNQFFLKLNSVTSEDTATYYC

SEQ ID NO: 34 - protein, 11 aa, Artificial Sequence
  FR4
WGQGTTLTVSS

SEQ ID NO: 35 - protein, 120 aa, Artificial Sequence
  Heavy chain variable (amino acid)
LEVKLEESGPGLVKPSQSLSLTCSVTGYSITSGYYWNWIRQFPGNKLEWMGFMSYDGSNNYNPSLKNRISITRDTSKNQFFLKLNSVTSEDTATYYCSRALAYYGNFDYWGQGTTLTVSS

SEQ ID NO: 36 - DNA, 360 bp, Artificial Sequence
  Heavy chain variable (nucleotide sequence)
cttgaggtaaagctggaggagtcaggacctggcctcgtgaaaccttctcagtctctgtctctcacctgctctgtcactggctactccatcaccagtggttattactggaactggatccggcaatttccaggaaacaaactggaatggatgggtttcatgagctacgacggtagcaataactacaacccatctctcaaaaatcgaatctccatcactcgtgacacatctaagaaccagtttttcctgaaattgaattctgtgacttctgaggacacagctacatattactgttcaagagctctagcctactatggtaactttgactactggggccaaggcaccactctcacagtctcctca

SEQ ID NO: 37 - protein, 6 aa, Artificial Sequence
  CDRL1
QNVNTN

SEQ ID NO: 38 - protein, 3 aa, Artificial Sequence
  CDRL2
SAS

SEQ ID NO: 39 - protein, 9 aa, Artificial Sequence
  CDRL3
QQYNTYPLT

SEQ ID NO: 40 - protein, 26 aa, Artificial Sequence
  FR1
DIVLTQTPKVMSTSVGDRVSVTCKAS

SEQ ID NO: 41 - protein, 17 aa, Artificial Sequence
  FR2
VAWYQQKPGQSPKALIY

SEQ ID NO: 42 - protein, 36 aa, Artificial Sequence
  FR3
SRYSGVPDRFTGSGSGTDFTLTISNVQSEDLADYFC

SEQ ID NO: 43 - protein, 10 aa, Artificial Sequence
  FR4
FAAGTKLELK

SEQ ID NO: 44 - protein, 107 aa, Artificial Sequence
  Light chain variable (amino acid)
DIVLTQTPKVMSTSVGDRVSVTCKASQNVNTNVAWYQQKPGQSPKALIYSASSRYSGVPDRFTGSGSGTDFTLTISNVQSEDLADYFCQQYNTYPLTFAAGTKLELK

SEQ ID NO: 45 - DNA, 322 bp, Artificial Sequence
  Light chain variable (nucleotide sequence)
gacattgtgctcacacaaactccaaaagtcatgtccacatcagtaggagacagggtcagcgtcacctgcaaggccagtcagaatgttaatactaatgtagcctggtatcaacagaaaccagggcaatctcctaaagcactcatttactcggcatcctcccggtacagtggagtccctgatcgcttcacaggcagtggatctgggacagatttcactctcaccatcagcaatgtgcagtctgaagacttggctgactatttctgtcagcaatataacacctatccgctcacgttcgctgctgggaccaagctggagctaaaac

SEQ ID NO: 46 - protein, 8 aa, Artificial Sequence
  CDRH1
GFTFSSFW

SEQ ID NO: 47 - protein, 10 aa, Artificial Sequence
  CDRH2
IRLKSNNYAT

SEQ ID NO: 48 - protein, 10 aa, Artificial Sequence
  CDRH3
IPPYYYTVDY

SEQ ID NO: 49 - protein, 25 aa, Artificial Sequence
  FR1
EVKLQESGGGLVQPGRSLKLSCVAS

SEQ ID NO: 50 - protein, 17 aa, Artificial Sequence
  FR2
MHWVRQSPEKGLEWVAE

SEQ ID NO: 51 - protein, 38 aa, Artificial Sequence
  FR3
HYAESVKGRFTISRDDSKSSVHLQMNNLRTEDTGIYYC

SEQ ID NO: 52 - protein, 11 aa, Artificial Sequence
  FR4
WGQGTSVTVSS

SEQ ID NO: 53 - protein, 119 aa, Artificial Sequence
  Heavy chain variable (amino acid)
EVKLQESGGGLVQPGRSLKLSCVASGFTFSSFWMHWVRQSPEKGLEWVAEIRLKSNNYATHYAESVKGRFTISRDDSKSSVHLQMNNLRTEDTGIYYCIPPYYYTVDYWGQGTSVTVSS

SEQ ID NO: 54 - DNA, 357 bp, Artificial Sequence
  Heavy chain variable (nucleotide sequence)
gaagtgaagctgcaggagtctggaggaggcttggtgcaacctggaagatccttgaaactctcctgtgttgcctctggattcactttcagtagtttctggatgcactgggtccgccagtctccagagaagggtcttgagtgggttgctgaaattcgattgaaatctaataattatgcaacacattatgcggagtctgtgaaagggaggttcaccatctcaagagatgattccaaaagtagcgtccacctgcaaatgaacaacttaagaactgaagacactggcatttattactgtatcccaccctattactatactgtggactactggggtcaaggaacctcagtcaccgtctcctca

SEQ ID NO: 55 - DNA, 323 bp, Artificial Sequence
  Light chain variable (nucleotide sequence)
gacattgtgatgacacaatctacagccatcctgtctgtgagtccaggagaaagagtcagtttctcctgcagggccagtcggagcattggcacaagcatacactggtatcagcaaagaacaaatggttctccaaggcttctcataaagtatgcttctgagtctatctctgggatcccttccaggtttagtggcagtggatcagggacagattttactcttcgcatcaacagtgtggagtctgaagatcttgcagattattactgtcaacaaagtgatagctggccaaccacgttcggaggggggaccaagctggaaataaaacg

SEQ ID NO: 56 - protein, 25 aa, Artificial Sequence
  FR1
QVKLQESGPELVKPGTSVRISCKAS

SEQ ID NO: 57 - protein, 17 aa, Artificial Sequence
  FR2
IHWVKQRPGQGLEWIGW

SEQ ID NO: 58 - protein, 121 aa, Artificial Sequence
  Heavy chain variable (amino acid)
QVKLQESGPELVKPGTSVRISCKASGYTFTSYYIHWVKQRPGQGLEWIGWIYPGNVNTKYNEKFKGKATLTADKSSSTAYMQLSSLTSEDSAVYFCARLGYYRYGGAMDYWGQGTSVTVSS

SEQ ID NO: 59 - DNA, 363 bp, Artificial Sequence
  Heavy chain variable (nucleotide sequence)
caggtgaagctgcaggagtcaggacctgagctggtgaagcctgggacttcagtgaggatatcctgcaaggcttctggctacaccttcacaagctactatatacattgggtgaagcagaggcctggacagggacttgagtggattggatggatttatcctggaaatgttaatactaagtacaatgagaagttcaagggcaaggccacactgactgcagacaaatcctccagcacagcctacatgcaactcagcagcctgacctctgaggactctgcggtctatttctgtgcaaggttgggctactataggtacgggggtgctatggactactggggtcaaggaacctcagtcaccgtctcctca

SEQ ID NO: 60 - protein, 6 aa, Artificial Sequence
  CDRL1
QDISKY

SEQ ID NO: 61 - protein, 9 aa, Artificial Sequence
  CDRL3
QQGNTLPRT

SEQ ID NO: 62 - protein, 26 aa, Artificial Sequence
  FR1
DIVLTQSPSSLSASLGDRVTITCRAS

SEQ ID NO: 63 - protein, 17 aa, Artificial Sequence
  FR2
LNWYQQKPDGTVKLLIY

SEQ ID NO: 64 - protein, 36 aa, Artificial Sequence
  FR3
TLHSGVPSRFSGSGSGTDFSLTITNLEQEDIATYFC

SEQ ID NO: 65 - protein, 107 aa, Artificial Sequence
  Light chain variable (amino acid)
DIVLTQSPSSLSASLGDRVTITCRASQDISKYLNWYQQKPDGTVKLLIYYTSTLHSGVPSRFSGSGSGTDFSLTITNLEQEDIATYFCQQGNTLPRTFGGGTKLEIK

SEQ ID NO: 66 - DNA, 322 bp, Artificial Sequence
  Light chain variable (nucleotide sequence)
gatattgtgctcacccaatctccatcctccctgtctgcctctctgggagacagagtcaccatcacttgcagggcaagtcaggacattagcaaatacttaaactggtatcaacagaaaccagatggaactgttaaactcctgatctactacacatcaacattacattcaggagtcccatcaaggttcagtggcagtgggtctgggacagatttttctctcaccattaccaacctggagcaagaagatattgccacttacttttgccaacagggcaatactcttcctcggacgttcggtggaggcaccaagctggaaatcaaac

SEQ ID NO: 67 - protein, 9 aa, Artificial Sequence
  CDRH1
GSSITSDYA

SEQ ID NO: 68 - protein, 7 aa, Artificial Sequence
  CDRH2
IGYSVGT

SEQ ID NO: 69 - protein, 13 aa, Artificial Sequence
  CDRH3
ARRPLYFYYAMDF

SEQ ID NO: 70 - protein, 26 aa, Artificial Sequence
  FR1
LEVKLEESGPGLLKPSQSLSLTCTVT

SEQ ID NO: 71 - protein, 17 aa, Artificial Sequence
  FR2
WNWIRQFPGNKLEWMGY

SEQ ID NO: 72 - protein, 38 aa, Artificial Sequence
  FR3
SHNPSLKGRISITRDTSKNQFFLHLNSVTTEDTAIYYC

SEQ ID NO: 73 - protein, 121 aa, Artificial Sequence
  Heavy chain variable (amino acid)
LEVKLEESGPGLLKPSQSLSLTCTVTGSSITSDYAWNWIRQFPGNKLEWMGYIGYSVGTSHNPSLKGRISITRDTSKNQFFLHLNSVTTEDTAIYYCARRPLYFYYAMDFWGQGTSVTVSS

SEQ ID NO: 74 - DNA, 363 bp, Artificial Sequence
  Heavy chain variable (nucleotide sequence)
cttgaggtgaagctggaggagtctggacctggcctgttgaaaccttctcagtctctgtccctcacctgcactgtcactggctcctcaatcaccagtgattatgcctggaactggatccggcagtttccaggaaacaaactggagtggatgggctacataggttacagtgttggcactagccacaacccatctctcaaaggtcgaatctctatcactcgagacacatccaagaaccagttcttcctgcacttgaattctgtgactactgaggacacagccatatattactgtgcaagacgccctctctacttctactatgctatggacttctggggtcaaggaacctcagtcaccgtctcctca

SEQ ID NO: 75 - protein, 10 aa, Artificial Sequence
  CDRL1
KSVSTSAYNF

SEQ ID NO: 76 - protein, 3 aa, Artificial Sequence
  CDRL2
LAS

SEQ ID NO: 77 - protein, 9 aa, Artificial Sequence
  CDRL3
QHSRELPYT

SEQ ID NO: 78 - protein, 26 aa, Artificial Sequence
  FR1
DIVLTQSPVSLAVSLGQRATISCRAS

SEQ ID NO: 79 - protein, 17 aa, Artificial Sequence
  FR2
MHWYQQKPGQPPKLLIY

SEQ ID NO: 80 - protein, 36 aa, Artificial Sequence
  FR3
NLESGVPARLSGSGSGTDFTLNIHPVEEEDAATYYC

SEQ ID NO: 81 - protein, 112 aa, Artificial Sequence
  Light chain variable (amino acid)
DIVLTQSPVSLAVSLGQRATISCRASKSVSTSAYNFMHWYQQKPGQPPKLLIYLASNLESGVPARLSGSGSGTDFTLNIHPVEEEDAATYYCQHSRELPYTFGGGTKLEIKR

SEQ ID NO: 82 - DNA, 335 bp, Artificial Sequence
  Light chain variable (nucleotide sequence)
gacattgtgctgacacagtctcctgtttctttagctgtatctctggggcagagggccaccatctcatgcagggccagcaaaagtgtcagtacctctgcctataattttatgcactggtaccagcagaaaccaggacagccacccaaactcctcatctatcttgcatccaacctagaatctggggtccctgccaggctcagtggcagtgggtctgggacagacttcaccctcaacatccatcctgtggaggaggaggatgctgcaacctattactgtcagcacagtagggagcttccgtacacgttcggaggggggaccaagctggaaataaaacg

SEQ ID NO: 83 - protein, 8 aa, Artificial Sequence
  CDRH1
GYTFTDYN

SEQ ID NO: 84 - protein, 8 aa, Artificial Sequence
  CDRH2
IYPYNGNS

SEQ ID NO: 85 - protein, 11 aa, Artificial Sequence
  CRH3
ASLYYLGYFDV

SEQ ID NO: 86 - protein, 25 aa, Artificial Sequence
  FR1
EVKLEQSGPELVKPGASVKISCKAS

SEQ ID NO: 87 - protein, 17 aa, Artificial Sequence
  FR2
MHWVRQPPGQSLEWIGF

SEQ ID NO: 88 - protein, 38 aa, Artificial Sequence
  FR3
GYNPNFKTKATLTMDISSSTAYMEVRNLTSEDSATYFC

SEQ ID NO: 89 - protein, 11 aa, Artificial Sequence
  FR4
WGAGTTVTVSS

SEQ ID NO: 90 - protein, 118 aa, Artificial Sequence
  Heavy chain variable (amino acid)
EVKLEQSGPELVKPGASVKISCKASGYTFTDYNMHWVRQPPGQSLEWIGFIYPYNGNSGYNPNFKTKATLTMDISSSTAYMEVRNLTSEDSATYFCASLYYLGYFDVWGAGTTVTVSS

SEQ ID NO: 91 - DNA, 354 bp, Artificial Sequence
  Heavy chain variable (nucleotide sequence)
gaagtgaagctggagcagtctggacctgagctggtgaaacctggggcctcagtgaagatatcctgcaaggcttctggatatacattcactgactacaacatgcactgggtgaggcagccccctggacagagccttgagtggattggatttatttatccttacaatggtaattctggctacaatccgaacttcaagaccaaggccacattgaccatggacatttcctccagcacagcctacatggaggtccgcaacctgacatctgaggattctgcgacctatttctgtgcaagtctttattacctcggatacttcgatgtgtggggcgcagggaccacggtcaccgtctcctca

SEQ ID NO: 92 - protein, 26 aa, Artificial Sequence
  FR1
DIVLTQSTAILSVSPGERVSFSCRAS

SEQ ID NO: 93 - protein, 108 aa, Artificial Sequence
  Light chain variable (amino acid)
DIVLTQSTAILSVSPGERVSFSCRASQSIGTSIHWYQQRTNGSPRLLIKYASESISGIPSRFSGSGSGTDFTLRINSVESEDLADYYCQQSDSWPTTFGGGTKLEIKR

SEQ ID NO: 94 - DNA, 323 bp, Artificial Sequence
  Light chain variable (nucleotide sequence)
gacattgtgctgacccagtctacagccatcctgtctgtgagtccaggagaaagagtcagtttctcctgcagggccagtcagagcattggcacaagcatacactggtatcagcaaagaacaaatggttctccaaggcttctcataaaatatgcttctgagtctatctctgggatcccttccaggtttagtggcagtggatcagggacagattttactcttcgcatcaacagtgtggagtctgaggatcttgcagattattactgtcagcaaagtgatagctggccaaccacgttcggaggggggaccaagctggaaataaaacg

SEQ ID NO: 95 - protein, 25 aa, Artificial Sequence
  FR1
EVQLEESGPELVKPGTSVRISCKAS

SEQ ID NO: 96 - protein, 121 aa, Artificial Sequence
  Heavy chain variable (amino acid)
EVQLEESGPELVKPGTSVRISCKASGYTFTSYYMHWVKQRPGQGLEWIGYIYPGNVNTKYNEKFKGKATLTADKSSSTAYMQLSSLTSEDSAVYFCARLGYYRYGGAMDYWGQGTSVTVSS

SEQ ID NO: 97 - DNA, 363 bp, Artificial Sequence
  Heavy chain variable (nucleotide sequence)
gaggttcagctggaggagtctggacctgagctggtgaagcctgggacttcagtgaggatatcctgcaaggcttctggctacaccttcacaagctactatatgcattgggtgaagcagaggcctggacagggacttgagtggattggatacatttatcctggaaatgttaatactaaatacaatgagaagttcaagggcaaggccacactgactgcagacaaatcctccagcacagcctacatgcaactcagcagcctgacctctgaggactctgcggtctatttctgtgcaaggttgggctactataggtacgggggtgctatggactactggggtcaaggaacctcagtcaccgtctcctca

SEQ ID NO: 98 - protein, 5 aa, Artificial Sequence
  CDRL1
SSVSY

SEQ ID NO: 99 - protein, 3 aa, Artificial Sequence
  CDRL2
DTS

SEQ ID NO: 100 - protein, 9 aa, Artificial Sequence
  CDRL3
FQGNKYPLT

SEQ ID NO: 101 - protein, 26 aa, Artificial Sequence
  FR1
DIVLTQTTAIMSASPGEKVTMTCSAS

SEQ ID NO: 102 - protein, 17 aa, Artificial Sequence
  FR2
IHWYQQKSSTSPKLWIY

SEQ ID NO: 103 - protein, 36 aa, Artificial Sequence
  FR3
KLASGVPGRFSGSGSGNSYSLTISSMEAEDVATYYC

SEQ ID NO: 104 - protein, 10 aa, Artificial Sequence
  FR4
FGAGTKLELK

SEQ ID NO: 105 - protein, 106 aa, Artificial Sequence
  Light chain variable (amino acid)
DIVLTQTTAIMSASPGEKVTMTCSASSSVSYIHWYQQKSSTSPKLWIYDTSKLASGVPGRFSGSGSGNSYSLTISSMEAEDVATYYCFQGNKYPLTFGAGTKLELK

SEQ ID NO: 106 - DNA, 319 bp, Artificial Sequence
  Light chain variable (nucleotide sequence)
gacattgtgctgacacaaactacagcaatcatgtctgcatctccaggggaaaaggtcaccatgacctgcagtgccagctcaagtgtaagttacatacactggtaccagcagaagtcaagcacctcccccaaactctggatttatgacacatccaaactggcttctggagtcccaggtcgcttcagtggcagtgggtctggaaactcttactctctcacgatcagcagcatggaggctgaagatgttgccacttactactgttttcaggggaataagtacccgctcacgttcggtgctgggaccaagttggagctgaaac

SEQ ID NO: 107 - protein, 8 aa, Artificial Sequence
  CDRH1
GFSLTSYG

SEQ ID NO: 108 - protein, 7 aa, Artificial Sequence
  CDRH2
IWAGGTT

SEQ ID NO: 109 - protein, 11 aa, Artificial Sequence
  CDRH3
ASYDYDGYFDV

SEQ ID NO: 110 - protein, 25 aa, Artificial Sequence
  FR1
QVKLQQSGPGLVAPSQSLSITCTVS

SEQ ID NO: 111 - protein, 17 aa, Artificial Sequence
  FR2
VHWVRQPPGKGLEWLGV

SEQ ID NO: 112 - protein, 38 aa, Artificial Sequence
  FR3
NYNSALMSRLSISKDNSKSQVFLKMNSLQTDDTAMYYC

SEQ ID NO: 113 - protein, 117 aa, Artificial Sequence
  Heavy chain variable (amino acid)
QVKLQQSGPGLVAPSQSLSITCTVSGFSLTSYGVHWVRQPPGKGLEWLGVIWAGGTTNYNSALMSRLSISKDNSKSQVFLKMNSLQTDDTAMYYCASYDYDGYFDVWGAGTTVTVSS

SEQ ID NO: 114 - DNA, 351 bp, Artificial Sequence
  Heavy chain variable (nucleotide sequence)
caggtcaagctgcagcagtcaggacctggcctggtggcgccctcacagagcctgtccatcacttgcactgtctctggcttttcattaaccagctatggtgtacactgggttcgccagcctccaggaaagggtctggagtggctgggagtcatatgggctggtggaaccacaaattataattcggctctcatgtctagactgagcatcagcaaagacaattccaagagccaagttttcttaaaaatgaacagtctgcaaactgatgacacagccatgtactactgtgccagttatgattacgacgggtacttcgatgtctggggcgcagggaccacggtcaccgtctcctca

SEQ ID NO: 115 - protein, 6 aa, Artificial Sequence
  CDRL1
QDIGSN

SEQ ID NO: 116 - protein, 3 aa, Artificial Sequence
  CDRL2
ATS

SEQ ID NO: 117 - protein, 9 aa, Artificial Sequence
  CDRL3
LQYISSPFT

SEQ ID NO: 118 - protein, 26 aa, Artificial Sequence
  FR1
DIVLTQTPSSLSASLGERVSLTCRAS

SEQ ID NO: 119 - protein, 17 aa, Artificial Sequence
  FR2
LNWLQQEPDGTIKRLIY

SEQ ID NO: 120 - protein, 36 aa, Artificial Sequence
  FR3
SLDPGVPKRFSGSRSGSDYSLTISSLESEDFVDYYC

SEQ ID NO: 121 - protein, 10 aa, Artificial Sequence
  FR4
FGSGTKLEIK

SEQ ID NO: 122 - protein, 107 aa, Artificial Sequence
  Light chain variable (amino acid)
DIVLTQTPSSLSASLGERVSLTCRASQDIGSNLNWLQQEPDGTIKRLIYATSSLDPGVPKRFSGSRSGSDYSLTISSLESEDFVDYYCLQYISSPFTFGSGTKLEIK

SEQ ID NO: 123 - protein, 322 aa, Artificial Sequence
  Light chain variable (nucleotide sequence)
GACATTGTGCTCACCCAGACTCCATCCTCCTTATCTGCCTCTCTGGGAGAAAGAGTCAGTCTCACTTGTCGGGCAAGTCAGGACATTGGTAGTAACTTAAACTGGCTTCAGCAGGAACCAGATGGAACTATTAAACGCCTGATCTACGCCACATCCAGTTTAGATCCAGGTGTCCCCAAAAGGTTCAGTGGCAGTAGGTCTGGGTCAGATTATTCTCTCACCATCAGCAGCCTTGAGTCTGAAGATTTTGTAGACTATTACTGTCTACAATATATTAGTTCTCCATTCACGTTCGGCTCGGGGACAAAGTTGGAAATAAAAC

SEQ ID NO: 124 - protein, 8 aa, Artificial Sequence
  CDRH1
GYSFTGYY

SEQ ID NO: 125 - protein, 8 aa, Artificial Sequence
  CDRH2
ISCYNGAT

SEQ ID NO: 126 - protein, 12 aa, Artificial Sequence
  CDRH3
ARGGTTATPFDY

SEQ ID NO: 127 - protein, 25 aa, Artificial Sequence
  FR1
QVKLEESGPELVKTGASVKISCKAS

SEQ ID NO: 128 - protein, 17 aa, Artificial Sequence
  FR2
IHWVKQSHGQSLEWIGY

SEQ ID NO: 129 - protein, 38 aa, Artificial Sequence
  FR3
RYNQKFKGKAAFTVDTSSSTAYLQFNSLTSEDSAIYYC

SEQ ID NO: 130 - protein, 119 aa, Artificial Sequence
  Heavy chain variable (amino acid)
QVKLEESGPELVKTGASVKISCKASGYSFTGYYIHWVKQSHGQSLEWIGYISCYNGATRYNQKFKGKAAFTVDTSSSTAYLQFNSLTSEDSAIYYCARGGTTATPFDYWGQGTTLTVSS

SEQ ID NO: 131 - DNA, 357 bp, Artificial Sequence
  Heavy chain variable (nucleotide sequence)
caggttaagctggaggagtcaggacctgagctagtgaagactggggcttcagtgaagatatcctgcaaggcttctggttactcattcactggttactacatacactgggtcaagcagagccatggacagagccttgagtggattggatatattagctgttacaacggtgctactagatataaccagaagttcaagggcaaggccgcatttactgtagacacatcctccagcacagcctacttacagttcaacagcctgacatctgaagactctgcgatctattactgtgcaagagggggtactacggctacgccctttgactactggggccaaggcaccactctcacagtctcctca

SEQ ID NO: 132 - protein, 6 aa, Artificial Sequence
  CDRL1
QDINNY

SEQ ID NO: 133 - protein, 3 aa, Artificial Sequence
  CDRL2
HAD

SEQ ID NO: 134 - protein, 10 aa, Artificial Sequence
  CDRL3
LQYDDFPPFT

SEQ ID NO: 135 - protein, 26 aa, Artificial Sequence
  FR1
DIVLTQTPSSMYAFLGERVTIICKAS

SEQ ID NO: 136 - protein, 17 aa, Artificial Sequence
  FR2
LNWVQQKPGKAPKTLIY

SEQ ID NO: 137 - protein, 36 aa, Artificial Sequence
  FR3
RLADGVPSRFSGSGSGQDYSLTISSLEYEDMGIYYC

SEQ ID NO: 138 - protein, 108 aa, Artificial Sequence
  Light chain variable (amino acid)
DIVLTQTPSSMYAFLGERVTIICKASQDINNYLNWVQQKPGKAPKTLIYHADRLADGVPSRFSGSGSGQDYSLTISSLEYEDMGIYYCLQYDDFPPFTFGSGTKLEIK

SEQ ID NO: 139 - DNA, 325 bp, Artificial Sequence
  Light chain variable (nucleotide sequence)
gacattgtgctgacccaaactccatcttccatgtatgcatttctaggagagagagtcaccatcatttgcaaggcgagtcaggacatcaataactatttaaactgggtccagcagaaaccagggaaagctcctaagaccctgatctatcatgcagacagattggcagatggggtcccatcaaggttcagtggcagtgggtctgggcaagattattctctcaccatcagcagcctggaatatgaagatatgggaatttattattgtctacagtatgatgactttcctccattcacgttcggctcggggacaaagttggaaataaaac

SEQ ID NO: 140 - protein, 8 aa, Artificial Sequence
  CDRH1
GFTFSSFG

SEQ ID NO: 141 - protein, 8 aa, Artificial Sequence
  CDRH2
ISGGSGTF

SEQ ID NO: 142 - protein, 9 aa, Artificial Sequence
  CDRH3
ARSTTVLDY

SEQ ID NO: 143 - protein, 25 aa, Artificial Sequence
  FR1
EVKLEQSGGGLVQPGGSRKLSCAAS

SEQ ID NO: 144 - protein, 17 aa, Artificial Sequence
  FR2
MHWVRQAPEKGLEWVAY

SEQ ID NO: 145 - protein, 38 aa, Artificial Sequence
  FR3
YYADTVKGRFTISRDNPKNTLFLQMTSLRSEDTAIYYC

SEQ ID NO: 146 - protein, 116 aa, Artificial Sequence
  Heavy chain variable (amino acid)
EVKLEQSGGGLVQPGGSRKLSCAASGFTFSSFGMHWVRQAPEKGLEWVAYISGGSGTFYYADTVKGRFTISRDNPKNTLFLQMTSLRSEDTAIYYCARSTTVLDYWGQGTTLTVSS

SEQ ID NO: 147 - DNA, 348 bp, Artificial Sequence
  Heavy chain variable (nucleotide sequence)
gaagttaagctggagcagtctgggggaggcttagtgcagcctggagggtcccggaaactctcctgtgcagcctctggattcactttcagtagctttgggatgcactgggttcgtcaggctccagagaaggggctggagtgggtcgcatatattagtggtggcagtggtaccttctactatgcagacacagtgaagggccgattcaccatctccagagacaatcccaagaacaccctattcctgcaaatgaccagtctaaggtctgaggacacggccatttattactgtgcaagatcgactacggttcttgactattggggccaaggcaccactctcacagtctcctca

SEQ ID NO: 148 - protein, 1400 aa, Artificial Sequence
  Full length RON protein amino acid sequence
MELLPPLPQSFLLLLLLPAKPAAGEDWQCPRTPYAASRDFDVKYVVPSFSAGGLVQAMVTYEGDRNESAVFVAIRNRLHVLGPDLKSVQSLATGPAGDPGCQTCAACGPGPHGPPGDTDTKVLVLDPALPALVSCGSSLQGRCFLHDLEPQGTAVHLAAPACLFSAHHNRPDDCPDCVASPLGTRVTVVEQGQASYFYVASSLDAAVAASFSPRSVSIRRLKADASGFAPGFVALSVLPKHLVSYSIEYVHSFHTGAFVYFLTVQPASVTDDPSALHTRLARLSATEPELGDYRELVLDCRFAPKRRRRGAPEGGQPYPVLRVAHSAPVGAQLATELSIAEGQEVLFGVFVTGKDGGPGVGPNSVVCAFPIDLLDTLIDEGVERCCESPVHPGLRRGLDFFQSPSFCPNPPGLEALSPNTSCRHFPLLVSSSFSRVDLFNGLLGPVQVTALYVTRLDNVTVAHMGTMDGRILQVELVRSLNYLLYVSNFSLGDSGQPVQRDVSRLGDHLLFASGDQVFQVPIQGPGCRHFLTCGRCLRAWHFMGCGWCGNMCGQQKECPGSWQQDHCPPKLTEFHPHSGPLRGSTRLTLCGSNFYLHPSGLVPEGTHQVTVGQSPCRPLPKDSSKLRPVPRKDFVEEFECELEPLGTQAVGPTNVSLTVTNMPPGKHFRVDGTSVLRGFSFMEPVLIAVQPLFGPRAGGTCLTLEGQSLSVGTSRAVLVNGTECLLARVSEGQLLCATPPGATVASVPLSLQVGGAQVPGSWTFQYREDPVVLSISPNCGYINSHITICGQHLTSAWHLVLSFHDGLRAVESRCERQLPEQQLCRLPEYVVRDPQGWVAGNLSARGDGAAGFTLPGFRFLPPPHPPSANLVPLKPEEHAIKFEYIGLGAVADCVGINVTVGGESCQHEFRGDMVVCPLPPSLQLGQDGAPLQVCVDGECHILGRVVRPGPDGVPQSTLLGILLPLLLLVAALATALVFSYWWRRKQLVLPPNLNDLASLDQTAGATPLPILYSGSDYRSGLALPAIDGLDSTTCVHGASFSDSEDESCVPLLRKESIQLRDLDSALLAEVKDVLIPHERVVTHSDRVIGKGHFGVVYHGEYIDQAQNRIQCAIKSLSRITEMQQVEAFLREGLLMRGLNHPNVLALIGIMLPPEGLPHVLLPYMCHGDLLQFIRSPQRNPTVKDLISFGLQVARGMEYLAEQKFVHRDLAARNCMLDESFTVKVADFGLARDILDREYYSVQQHRHARLPVKWMALESLQTYRFTTKSDVWSFGVLLWELLTRGAPPYRHIDPFDLTHFLAQGRRLPQPEYCPDSLYQVMQQCWEADPAVRPTFRVLVGEVEQIVSALLGDHYVQLPATYMNLGPSTSHEMNVRPEQPQFSPMPGNVRRPRPLSEPPRPT

SEQ ID NO: 149 - protein, 933 aa, Artificial Sequence
  Full length RON extracellular domain (ECD) amino acid sequence
EDWQCPRTPYAASRDFDVKYVVPSFSAGGLVQAMVTYEGDRNESAVFVAIRNRLHVLGPDLKSVQSLATGPAGDPGCQTCAACGPGPHGPPGDTDTKVLVLDPALPALVSCGSSLQGRCFLHDLEPQGTAVHLAAPACLFSAHHNRPDDCPDCVASPLGTRVTVVEQGQASYFYVASSLDAAVAASFSPRSVSIRRLKADASGFAPGFVALSVLPKHLVSYSIEYVHSFHTGAFVYFLTVQPASVTDDPSALHTRLARLSATEPELGDYRELVLDCRFAPKRRRRGAPEGGQPYPVLRVAHSAPVGAQLATELSIAEGQEVLFGVFVTGKDGGPGVGPNSVVCAFPIDLLDTLIDEGVERCCESPVHPGLRRGLDFFQSPSFCPNPPGLEALSPNTSCRHFPLLVSSSFSRVDLFNGLLGPVQVTALYVTRLDNVTVAHMGTMDGRILQVELVRSLNYLLYVSNFSLGDSGQPVQRDVSRLGDHLLFASGDQVFQVPIQGPGCRHFLTCGRCLRAWHFMGCGWCGNMCGQQKECPGSWQQDHCPPKLTEFHPHSGPLRGSTRLTLCGSNFYLHPSGLVPEGTHQVTVGQSPCRPLPKDSSKLRPVPRKDFVEEFECELEPLGTQAVGPTNVSLTVTNMPPGKHFRVDGTSVLRGFSFMEPVLIAVQPLFGPRAGGTCLTLEGQSLSVGTSRAVLVNGTECLLARVSEGQLLCATPPGATVASVPLSLQVGGAQVPGSWTFQYREDPVVLSISPNCGYINSHITICGQHLTSAWHLVLSFHDGLRAVESRCERQLPEQQLCRLPEYVVRDPQGWVAGNLSARGDGAAGFTLPGFRFLPPPHPPSANLVPLKPEEHAIKFEYIGLGAVADCVGINVTVGGESCQHEFRGDMVVCPLPPSLQLGQDGAPLQVCVDGECHILGRVVRPGPDGVPQST

SEQ ID NO: 150 - DNA, 18 bp, Artificial Sequence
  RON KO sequencing primer (Forward)
ggtccgctatcttggggc

SEQ ID NO: 151 - DNA, 20 bp, Artificial Sequence
  RON KO sequencing primer (Reverse)
ctgggcaccacgtacttcac

SEQ ID NO: 152 - DNA, 19 bp, Artificial Sequence
  RON KO gRNA spacer
ggcgggaggagctccatcg

(19) United States
(12) Patent Application Publication
(10) Pub. No.: US 2022/0372125 A1
(43) Pub. Date: Nov. 24, 2022

(54) Antigen Specific Binding Domains and Antibody Molecules

(71) Applicants: Aslan Pharmaceuticals Pte. Ltd., Singapore (SG); Agency for Science, Technology and Research, Singapore (SG)

(72) Inventors: Xin Yu Koh, Singapore (SG); David Philip Lane, Singapore (SG)

(21) Appl. No.: 17/766,131

(22) PCT Filed: Oct. 2, 2020
(86) PCT No.: PCT/SG2020/050557

§ 371 Date: Apr. 1, 2022

(30) Foreign Application Priority Data
Oct. 2, 2019 (SG) 10201909236R

(51) Int. Cl. C07K 16/22; A61K 47/68; A61P 35/00; A61K 51/10

(52) U.S. Cl. CPC C07K 16/22; A61K 47/6845; A61P 35/00; A61K 51/1021; A61K 2039/505

(57) ABSTRACT

The present disclosure relates to an antigen specific binding domain which binds to RON (Macrophage Stimulating Protein Receptor or Recepteur d'Origine Nantais). The disclosure also extends to chimeric antigen receptors (and a cell expressing the same), antibody molecules (including full length antibodies and fragments thereof, as well as antibody conjugates) containing the antigen binding domains disclosed herein. Also disclosed herein are pharmaceutical compositions comprising the cells, antibody molecules as disclosed herein. The present disclosure also refers to the use of the antigen binding domains, the chimeric antigen receptors, the antibody molecules and the pharmaceutical compositions disclosed herein in therapy, more particularly in treating cancer. Also disclosed herein are radiolabelled antibody conjugates comprising the antigen binding domains as disclosed, and their use in methods of treatment or diagnosis.

### CROSS-REFERENCE TO RELATED APPLICATIONS

This application claims the benefit of priority of Singapore patent application No. 10201909236R, filed on 2 Oct. 2019, the contents of it being hereby incorporated by reference in its entirety for all purposes.

### FIELD OF THE INVENTION

The present invention relates to the field of biotechnology. In particular, the present invention refers to antigen binding domains that bind to RON (Macrophage stimulating protein receptor or Recepteur d'Origine Nantais), and antibody molecules comprising the same. The present invention also refers to methods of treating, diagnosing or evaluating diseases using the antigen binding domains disclosed herein.

### BACKGROUND

The RON receptor tyrosine kinase (also known as Macrophage Stimulating Protein Receptor or Recepteur d'Origine Nantais) is expressed at high levels at the surface on many tumor cells of epithelial origin and antibodies to RON have shown therapeutic potential in preclinical models both as intact antibodies and as antibody drug conjugates. RON is a member of the MET receptor family and its sole ligand is the macrophage stimulating protein (MSP). Overexpressed RON is an oncogenic driver and small molecule inhibitors of the RON kinase and antibodies to the extracellular domain of RON have shown anti-tumor activity in a variety of pre-clinical models. RON is expressed on myeloid cells and recent studies have shown that the anti-tumor activity of anti-CTLA4 antibodies is enhanced in RON knock out mice suggesting that inhibition of RON may enhance the activity of anti-CTLA4 treatment in enhancing host anti-tumor immunity. Narnatumab, a humanized monoclonal to RON entered phase I clinical trials but failed for lack of efficacy in part because the antibody could not be given at high doses due to solubility issues. There is therefore a need to provide anti-RON antibodies with improved binding affinities or biochemical properties for use in cancer therapy.

A key criterion in the development of antibody therapeutics for the treatment of cancer is to show preclinical evidence that the antibody therapeutic drug has remarkable anti-tumour efficacies with little toxicities. A novel image guided approach to monitor potential unspecific binding caused by the antibody drug would be through the use of companion diagnostics. When labelled with a radiolabel, the antibody PET (positron emission tomography) imaging agent can act as a tool to allow global tracking of the antigen expression in tumours or normal tissues in vivo, both as a predictor of therapeutic drug responses and any on-target off-tumour toxicities. In addition, in comparison to the traditional way of using immunohistochemistry staining of selected tissue biopsies as an indication of local tumour antigen expression, immunoPET companion diagnostic agents allow for a more complete and reliable view of antigen expression across the tumours in vivo, taking into considering any heterogeneity that might often be present in tumours. Companion diagnostics can also be used as a predictor of uptake of antibodies into tumour tissues or organs as well as to detect possible differential antigen expression in metastases. They are often used to provide valuable information on the pharmacokinetics and clearance of the antibodies in vivo. Thus, there is also a need to provide anti-RON antibodies with binding affinities for generating useful immunoPET agents for detecting/monitoring RON expression in vivo.

### SUMMARY

The present invention addresses the above mentioned needs by providing novel antigen binding domains having binding affinities for RON, which further enables the generation of chimeric antigen receptors, and anti-RON antibodies. The antibodies are useful as therapeutic or diagnostic agents for cancer.

In one aspect, the present disclosure refers to an antigen specific binding domain which binds to RON (Macrophage stimulating protein receptor or Recepteur d'Origine Nantais), comprising a light chain variable region (VL) and a heavy chain variable region (VH), wherein the heavy chain variable region (VH) comprises Complementarity Determining Regions (CDRs) CDRH1, CDRH2, CDRH3, and the light chain variable region comprises CDRs CDRL1, CDRL2, and CDRL3; wherein:
i. CDRH1 is selected from the group consisting of SEQ ID NO: 140, 10, 28, 46, 67, 83, 107, 124, and a CDRH1 differing from any one of the same in that 1 or 2 amino acids are replaced, deleted or added;
ii. CDRH2 is selected from the group consisting of SEQ ID NO: 141, 11, 29, 47, 68, 84, 108, 125, and a CDRH2 differing from any one of the same in that 1 or 2 amino acids are replaced, deleted or added;
iii. CDRH3 is selected from the group consisting of SEQ ID NO: 142, 12, 30, 48, 69, 85, 109, 126, and a CDRH3 differing from any one of the same in that 1 or 2 amino acids are replaced, deleted or added;
iv. CDRL1 is selected from the group consisting of SEQ ID NO: 132, 1, 19, 37, 60, 75, 98, 115, and a CDRL1 differing from any one of the same in that 1 or 2 amino acids are replaced, deleted or added;
v. CDRL2 is selected from the group consisting of SEQ ID NO: 133, 2, 20, 38, 76, 99, 116, and a CDRL2 differing from any one of the same in that 1 or 2 amino acids are replaced, deleted or added; and
vi. CDRL3 is selected from the group consisting of SEQ ID NO: 134, 3, 21, 39, 61, 77, 100, 117, and a CDRL3 differing from any one of the same in that 1 or 2 amino acids are replaced, deleted or added.

In another aspect, the present disclosure refers to a chimeric antigen receptor comprising the antigen specific binding domain as disclosed herein.

In another aspect, the present disclosure refers to a cell expressing the chimeric antigen receptor as disclosed herein, optionally wherein the cell is selected from a T cell (such as a cytotoxic T cell), an Natural Killer (NK) cell and a Natural Killer T (NKT) cell.

In another aspect, the present disclosure refers to an antibody molecule comprising the antigen specific binding domain as disclosed herein.

In another aspect, the present disclosure refers to a polynucleotide encoding the antigen specific binding domain, the chimeric antigen receptor, or the antibody molecule as disclosed herein.

In a further aspect, the present disclosure refers to a pharmaceutical composition comprising the antigen specific binding domain, the chimeric antigen receptor, the cell, the antibody molecule, the polynucleotide as disclosed herein, optionally wherein the pharmaceutical composition further comprises an excipient, diluent and/or carrier.

In a further aspect, the present disclosure refers to the antigen specific binding domain, the chimeric antigen receptor, the cell, the antibody molecule, the polynucleotide, or the pharmaceutical composition as disclosed herein for use in therapy.

In yet another aspect, the present disclosure refers to the use of the antigen specific binding domain, the chimeric antigen receptor, the cell, the antibody molecule, the polynucleotide, or the pharmaceutical composition as disclosed herein in the manufacture of a medicament for the treatment of cancer.

In still another aspect, the present disclosure refers to a method of treating a patient for cancer comprising administering a therapeutically effective amount of the antigen specific binding domain, the chimeric antigen receptor, the cell, the antibody molecule, the polynucleotide, or the pharmaceutical composition as disclosed herein.

In a further aspect, the present disclosure refers to a radiolabelled antibody conjugate comprising an antibody or antigen binding fragment thereof that binds RON (Macrophage-stimulating protein receptor, or Recepteur d'Origine Nantais), and a positron emitter, wherein the antibody or antigen binding fragment thereof comprises an antigen specific binding domain as disclosed herein.

In another aspect, the present disclosure refers to a method of imaging a tissue that expresses RON (Macrophage-stimulating protein receptor, or Recepteur d'Origine Nantais), comprising administering a radiolabelled antibody conjugate as disclosed herein; and visualizing RON expression by positron emission tomography (PET) imaging.

In yet another aspect, the present disclosure refers to method for treating a tumour comprising:

(a) selecting or providing a subject with a solid tumour;
(b) determining that the solid tumour is RON-positive; and
(c) administering one or more doses of a RON inhibitor to the subject;
wherein step (b) comprises: (i) administering a radiolabelled antibody conjugate as disclosed herein to the subject in need thereof; and (ii) imaging localization of the radiolabelled antibody conjugate in the tumour by positron emission tomography (PET) imaging, wherein presence of the radiolabelled antibody conjugate in the tumour indicates that the tumour is RON-positive.

In another aspect, the present disclosure refers to an antibody molecule which cross-blocks or binds the same epitope as an antibody molecule comprising a VH/VL pair selected from SEQ ID NO: 8 and 25, SEQ ID NO: 16 and 25, SEQ ID NO: 34 and 43, SEQ ID NO: 51 and 54, SEQ ID NO: 63 and 71, SEQ ID NO: 79 and 87, SEQ ID NO: 95 and 98, SEQ ID NO: 106 and 114, SEQ ID NO: 121 and 130 or SEQ ID NO: 142 and 150.

In another aspect, the present disclosure refers to a method of monitoring a cancer patient using an antibody molecule as disclosed herein, wherein the method comprises the steps of:
i. using a labelled form of the antibody molecule to access the cancer, in particular a tumour, at a first time point,
ii. using a labelled form of the antibody molecule to access the cancer, in particular a tumour, at least a second time point, and
iii. comparing the results from the two or more time points to evaluate the status of the cancer.

### BRIEF DESCRIPTION OF THE FIGURES

FIG. 1 Shows schematic representations of RON protein domains. Schematic Representations of RON structure including some “isoforms” are shown. General structures of RON are illustrated on the left. The α- and β-chains are indicated. Deleted regions in individual variants are marked with arrows. TM, transmembrane segment; MRS, maturation-related sequences; and TK, tyrosine kinase domain. The RON extracellular sequences contain several functional motifs including a sema domain followed by a cysteine-rich hinge (“plexin-semaphorin-integrin”, or “PSI”) and three immunoglobulin-plexin-transcription (IPT) units. The sema domain stretches in both α and β chains and is known to contain high affinity binding site for MSP (macrophage-stimulating protein). The antibodies disclosed in the present application target the α chain of the RON protein. This figure is extracted from Yao, H., Zhou, Y., Ma, Q. et al. Mol Cancer 10, 82 (2011).

FIG. 2 Shows a schematic representation of the antigen used for immunization. The antigen corresponds to the extracellular domain of the RON protein, which extends from amino acid Gly25 to Thr 957, and covers the SEMA, PSI and IPT units. The antigen is expressed in, and purified from mammalian cells.

FIG. 3 Shows the IC₅₀ results for the anti-RON antibodies of the present disclosure (A) Antibody 3G4 (B) Antibody 3F8 (C) Antibody 6A9 (D) Antibody 7H11 (E) Antibody 9F2 (F) Antibody 10G1.

FIG. 4 Shows the binding affinities of the anti-RON antibodies of the present disclosure as determined by KinExA.

FIG. 5 Shows the screening and characterization of RON antibodies via ELISA, cell staining, immunoprecipitation and flow cytometry. A) A total of eight antibody clones were identified from the ELISA screen. Mammalian expressed extracellular RON antigen (His-tag) was coated on ELISA plates and His-tagged protein was used as a negative control Immunized mouse serum (1:1000), 1B5A9 (commercial anti-RON antibody) and anti-His (commercial) was used as positive control antibodies and mIgG was used as a negative control. Absorbance was read at 650 nm using Envision plate reader (Perkin Elmer). B) Confocal images showing immunofluorescence staining of HCT116 cells and T47D cells using RON antibodies. Cells were stained with DAPI (blue) for nuclei visualisation and antibodies (green) using anti-mouse secondary antibody conjugated with Alexa Fluor 488. Polyclonal mouse IgG was used as a negative control. Scale bar: 50 μm C) Endogenous RON was immunoprecipitated using anti-RON antibodies. Narnatumab was used as a positive control and mouse IgG was used as a negative control. Eluted protein was resolved on an SDS page gel and homemade anti-alpha RON antibody 6E6 was used to probe for immunoprecipitated RON. D) Anti-RON antibodies were used to stain live cells and fixed T47D cells (lighter peak) and T47D RON KO cells (darker peak) in flow cytometry. Anti-mouse secondary antibody conjugated with Alexa Fluor 488 (Invitrogen) was used for visualization of antibody staining.

FIG. 6 Shows the KinExA determined binding affinities of anti-RON antibodies on mammalian expressed extracellular RON antigens. Titration curves were generated using indicated fixed concentrations of extracellular RON antigens measured by calibration free concentration analysis (CFCA). Increasing concentrations of RON antibodies were titrated for measurement of binding. Apparent Kd values were determined from an average of the two curves and plotted in table shown.

FIG. 7 Shows that Anti-RON antibodies exerts antagonistic effects on MSP induced increase in levels of phosphorylated ERK. A) RON expressing cell lines were probed for levels of phosphorylated ERK following stimulation by MSP. 293FT cell line was used as a negative control. Phospho-p44/42 MAPK (Erk1/2) (Thr202/Tyr204) Antibody #9101 (Cell signalling technologies) was used as a primary antibody. Secondary anti-rabbit-HRP (Jackson Laboratories) was used to visualize western binding. β-actin served as a loading control B) T47D cells and T47D RON knockout cells were treated with 10 μg/ml anti-RON antibodies for an hour followed by MSP for half an hour. Cells were harvested and probed for p-ERK levels with AlphaLISA surefire Ultra p-ERK Assay Kit (Perkin Elmer). C) T47D cells were treated with increasing concentrations (333 nM to 4 fM) anti-RON antibodies for an hour followed by MSP for half an hour. Cells were harvested and probed for p-ERK levels with AlphaLISA surefire Ultra p-ERK Assay Kit (Perkin Elmer). IC50 titration curves were plotted and calculated IC50 values were drawn in the table below. Wortmannin was used as a positive control and mouse IgG was used as negative control. Mouse serum refers to the polyclonal serum from mice immunized with human RON ECD antigen. D) EC50 values and ranking of the antibodies in pERK inhibition assays. E) T47D cells were treated with increasing concentrations of 10G1 and Narnatumab antibodies for an hour followed by MSP for half an hour. p-ERK levels were probed and EC50 values were calculated in the same ways as in panel C).

FIG. 8 Shows the antibody dependent cellular cytotoxicity activities of anti-RON antibodies tested in vitro. A) All eight anti-RON antibodies (chimeric mouse VH with human Fc) were tested for their abilities to induce antibody dependent cellular cytotoxicity (ADCC). Humanized Trastuzumab was used as a positive control. Target T47D cancer cells were seeded overnight for baseline measurement. Purified human NK were chosen as effector cells an E:T ratio of 10:1. Varying concentrations of antibodies (0.1 pg/ml to 1 μg/ml) were tested on target cells. Impedance based assay xCELLigence (Acea Bio) was used for quantification of ADCC activities. Percentage of cytolysis plot was calculated and plotted by xCELLigence Immunotherapy software (Acea Bio). Data plotted starts from addition of effector cells and antibodies. Representative graphs of 3G4, 3F8 and 10G1 are shown. B) Normalized EC₅₀ titration curve data of percentage cytolysis of individual antibodies and Trastuzumab at 24 hours following addition of effector cells and antibodies. EC₅₀ values were noted and summarized in (C).

FIG. 9 Shows that RON antibodies selectively targets RON-positive HT29 tumor xenografts. A) Whole-body coronal and axial small-animal PET/CT images of RON positive HT29 (right flank) and RON negative HCT116 (left flank) tumor-bearing mice at 24, 48, and 72 h after intravenous injection of ⁸⁹Zr-labelled 10G1 and 3F8. B) Standardized uptake (SUV) in RON positive and RON negative tumors of ⁸⁹Zr-10G1 and ⁸⁹Zr-3F8 over 72 h (n=2, error bars=SD). C) Comparison of ⁸⁹Zr-10G1 and ⁸⁹Zr-3F8 uptake in RON positive and RON negative tumors, presented as percent injected dose per gram tumor (n=4, error bars=SD). D)⁸⁹Zr-10G1 and ⁸⁹Zr-3F8 uptake in tumors and major organs 72 h p.i., presented as percent injected dose per gram of tissue (n=4, error bars=SD). E) In-vitro assessment of ⁸⁹Zr-10G1 and ⁸⁹Zr-3F8 on RON positive and RON negative cell lines in ELISA (n=2-3, error bars=SD).

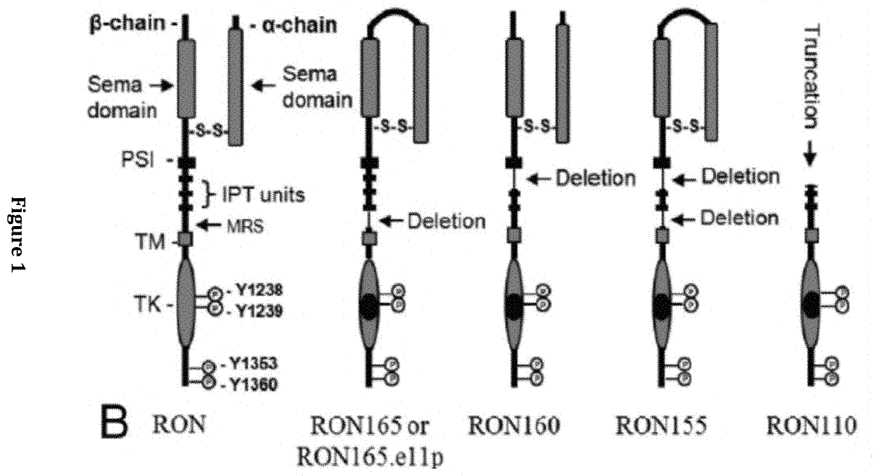

### DETAILED DESCRIPTION

Macrophage stimulating protein receptor (RON) is a c-MET-related tyrosine kinase receptor which transduces signals from the extracellular matrix into the cytoplasm when engaged by the ligand MSP. This signalling stimulates the intracellular domain of RON and provides active sites for engaging downstream signalling molecules, such as PIK3R1, PLCG1 or GAB1. Thus RON is part of the signalling cascade. The human RON receptor tyrosine kinase has the UniProt number Q04912 and the murine protein has the number is Q62190. The receptor is encoded in humans by gene MST1R.

RON is expressed at high levels at the surface on many tumor cells of epithelial origin and antibodies to RON have shown therapeutic potential both as intact antibodies and as antibody drug conjugates. Antigen binding domains that show high specificity and affinity towards RON would be highly useful in the generation and engineering of antibodies, antibody fragments, chimeric antigen receptors, antibody conjugates, and diagnostic/imaging reagents.

Therefore, in a first aspect, the present disclosure provides an antigen specific binding domain which binds to RON (Macrophage stimulating protein receptor or Recepteur d'Origine Nantais), comprising a light chain variable region (VL) and a heavy chain variable region (VH), wherein the heavy chain variable region (VH) comprises Complementarity Determining Regions (CDRs) CDRH1, CDRH2, CDRH3, and the light chain variable region comprises CDRs CDRL1, CDRL2, and CDRL3; wherein:

i. CDRH1 includes, but is not limited to any one of SEQ ID NO: 140, 10, 28, 46, 67, 83, 107, 124, or an amino acid sequence differing from any one of the same in that 1 or 2 amino acids are replaced, deleted or added;
ii. CDRH2 includes, but is not limited to any one of SEQ ID NO: 141, 11, 29, 47, 68, 84, 108, 125, or an amino acid sequence differing from any one of the same in that 1 or 2 amino acids are replaced, deleted or added;
iii. CDRH3 includes, but is not limited to any one of SEQ ID NO: 142, 12, 30, 48, 69, 85, 109, 126, or an amino acid sequence differing from any one of the same in that 1 or 2 amino acids are replaced, deleted or added;
iv. CDRL1 includes, but is not limited to any one of SEQ ID NO: 132, 1, 19, 37, 60, 75, 98, 115, and or an amino acid sequence differing from any one of the same in that 1 or 2 amino acids are replaced, deleted or added;
v. CDRL2 includes, but is not limited to any one of SEQ ID NO: 133, 2, 20, 38, 76, 99, 116, or an amino acid sequence differing from any one of the same in that 1 or 2 amino acids are replaced, deleted or added; and
vi. CDRL3 includes, but is not limited to any one of SEQ ID NO: 134, 3, 21, 39, 61, 77, 100, 117, or an amino acid sequence differing from any one of the same in that 1 or 2 amino acids are replaced, deleted or added.

In one example, the antigen specific binding domain binds with the extracellular RON protein. In one example, the antigen specific binding domain binds with the extracellular human RON protein.

As used herein, the term “antigen specific binding domain” refers to a binding domain that can bind to an antigen with a degree of specificity. The antigen specific binding domain can be part of or taken from an antibody (such as an immunoglobulin) or a non-antibody (such as a chimeric antigen receptor). In some examples, the antigen specific binding domain is part of a Fab (Fragment antigen-binding) of an antibody. In some other examples, the antigen specific binding domain is part of an extracellular antigen binding domain of a chimeric antigen receptor or a T-cell receptor. In some examples, the antigen specific binding domain is part of an antibody, wherein the antigen specific binding domain is formed the light chain variable region (VL) and a heavy chain variable region (VH). In some examples, the antigen specific binding domain is formed by a single peptide chain, for example in the case of a single chain variable fragment (scFv). It should be noted that even in such case wherein only a single peptide or peptide chain is involved in forming the antigen specific binding domain, the terms “VL” and “VH” may still be used to refer to the regions corresponding to (or derived from) the VL and VH of an antibody.

The term “an antigen specific binding domain which binds to RON (Macrophage stimulating protein receptor or Recepteur d'Origine Nantais)” may also be referred to as “a RON specific binding domain”.

CDRs as referred to herein are defined by the VBASE2 software: Retter I, Althaus H H, Munch R, Müller W: VBASE2, an integrative V gene database. Nucleic Acids Res. 2005 Jan. 1; 33 (Database issue):D671-4. In a specific example, the CDRs are defined by Kabat numbering as defined below.

CDRs of the heavy chain variable region are located at residues 31-35 (CDR-H1), residues 50-65 (CDR-H2) and residues 95-102 (CDR-H3) according to the Kabat numbering system. However, according to Chothia (Chothia, C. and Lesk, A. M. J. Mol. Biol., 196, 901-917 (1987)), the loop equivalent to CDR-H1 extends from residue 26 to residue 32. Thus unless indicated otherwise ‘CDR-H1’ as employed herein is intended to refer to residues 26 to 35, as described by a combination of the Kabat numbering system and Chothia's topological loop definition.

The CDRs of the light chain variable domain are located at residues 24-34 (CDR-L1), residues 50-56 (CDR-L2) and residues 89-97 (CDR-L3) according to the Kabat numbering system.

The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence.

In a specific example of the antigen specific binding domain,

i. CDRH1 includes, but is not limited to any one of SEQ ID NO: 140, 10, 28, 46, 67, 83, 107, and 124;
ii. CDRH2 includes, but is not limited to any one of SEQ ID NO: 141, 11, 29, 47, 68, 84, 108, and 125;
iii. CDRH3 includes, but is not limited to any one of SEQ ID NO: 142, 12, 30, 48, 69, 85, 109, and 126;
iv. CDRL1 includes, but is not limited to any one of SEQ ID NO: 132, 1, 19, 37, 60, 75, 98, and 115;
v. CDRL2 includes, but is not limited to any one of SEQ ID NO: 133, 2, 20, 38, 76, 99, and 116; and
vi. CDRL3 includes, but is not limited to any one of SEQ ID NO: 134, 3, 21, 39, 61, 77, 100, and 117.

In another example of the antigen binding domain, the CDRs of the light chain variable region of the antigen specific binding domain as disclosed herein include, but are not limited to any one of

### (a) the CDRL1 is SEQ ID NO: 132, the CDRL2 is SEQ ID NO: 133, the CDRL3 is SEQ ID NO: 134;

### (b) the CDRL1 is SEQ ID NO: 1, the CDRL2 is SEQ ID NO: 2, the CDRL3 is SEQ ID NO: 3;

### (c) the CDRL1 is SEQ ID NO: 19, the CDRL2 is SEQ ID NO: 20, the CDRL3 is SEQ ID NO: 21;

### (d) the CDRL1 is SEQ ID NO: 37, the CDRL2 is SEQ ID NO: 38, the CDRL3 is SEQ ID NO: 39;

### (e) the CDRL1 is SEQ ID NO: 60, the CDRL2 is SEQ ID NO: 20, the CDRL3 is SEQ ID NO: 61;

### (f) the CDRL1 is SEQ ID NO: 75, the CDRL2 is SEQ ID NO: 76, the CDRL3 is SEQ ID NO: 77;

### (g) the CDRL1 is SEQ ID NO: 98, the CDRL2 is SEQ ID NO: 99, the CDRL3 is SEQ ID NO: 100; and

### (h) the CDRL1 is SEQ ID NO: 115, the CDRL2 is SEQ ID NO: 116, the CDRL3 is SEQ ID NO: 117;

In another example, the CDRs of the heavy chain variable region of the antigen specific binding domain include, but are not limited to any one of:

### (a) the CDRH1 is SEQ ID NO: 140, the CRDH2 is SEQ ID NO: 141, the CRDH3 is SEQ ID NO: 142;

### (b) the CDRH1 is SEQ ID NO: 10, the CRDH2 is SEQ ID NO: 11, the CRDH3 is SEQ ID NO: 12;

### (c) the CDRH1 is SEQ ID NO: 28, the CRDH2 is SEQ ID NO: 29, the CRDH3 is SEQ ID NO: 30;

### (d) the CDRH1 is SEQ ID NO: 46, the CRDH2 is SEQ ID NO: 47, the CRDH3 is SEQ ID NO: 48;

### (e) the CDRH1 is SEQ ID NO: 67, the CRDH2 is SEQ ID NO: 68, the CRDH3 is SEQ ID NO: 69;

### (f) the CDRH1 is SEQ ID NO: 83, the CRDH2 is SEQ ID NO: 84, the CRDH3 is SEQ ID NO: 85;

### (g) the CDRH1 is SEQ ID NO: 107, the CRDH2 is SEQ ID NO: 108, the CRDH3 is SEQ ID NO: 109;

and

### (h) the CDRH1 is SEQ ID NO: 124, the CRDH2 is SEQ ID NO: 125, the CRDH3 is SEQ ID NO: 126.

In another example, the CDRs of the antigen specific binding domain include, but are not limited to any one of:

### (a) the CDRL1 is SEQ ID NO: 132, the CDRL2 is SEQ ID NO: 133, the CDRL3 is SEQ ID NO: 134, the CDRH1 is SEQ ID NO: 140, the CRDH2 is SEQ ID NO: 141, and the CRDH3 is SEQ ID NO: 142;

### (b) the CDRL1 is SEQ ID NO: 1, the CDRL2 is SEQ ID NO: 2, the CDRL3 is SEQ ID NO: 3, the CDRH1 is SEQ ID NO: 10, the CRDH2 is SEQ ID NO: 11, and the CRDH3 is SEQ ID NO: 12;

### (c) the CDRL1 is SEQ ID NO: 19, the CDRL2 is SEQ ID NO: 20, the CDRL3 is SEQ ID NO: 21 the CDRH1 is SEQ ID NO: 28, the CRDH2 is SEQ ID NO: 29, the CRDH3 is SEQ ID NO: 30;

### (d) the CDRL1 is SEQ ID NO: 37, the CDRL2 is SEQ ID NO: 38, the CDRL3 is SEQ ID NO: 39, the CDRH1 is SEQ ID NO: 46, the CRDH2 is SEQ ID NO: 47, and the CRDH3 is SEQ ID NO: 48;

### (e) the CDRL1 is SEQ ID NO: 60, the CDRL2 is SEQ ID NO: 20, the CDRL3 is SEQ ID NO: 61, the CDRH1 is SEQ ID NO: 67, the CRDH2 is SEQ ID NO: 68, and the CRDH3 is SEQ ID NO: 69;

### (f) the CDRL1 is SEQ ID NO: 75, the CDRL2 is SEQ ID NO: 76, the CDRL3 is SEQ ID NO: 77, the CDRH1 is SEQ ID NO: 83, the CRDH2 is SEQ ID NO: 84, and the CRDH3 is SEQ ID NO: 85;

### (g) the CDRL1 is SEQ ID NO: 98, the CDRL2 is SEQ ID NO: 99, the CDRL3 is SEQ ID NO: 100, the CDRH1 is SEQ ID NO: 107, the CRDH2 is SEQ ID NO: 108, and the CRDH3 is SEQ ID NO: 109;

### (h) the CDRL1 is SEQ ID NO: 115, the CDRL2 is SEQ ID NO: 116, the CDRL3 is SEQ ID NO: 117, the CDRH1 is SEQ ID NO: 124, the CRDH2 is SEQ ID NO: 125, and the CRDH3 is SEQ ID NO: 126.

In another example, the heavy chain variable region of the antigen specific binding domain of the first aspect comprises a sequence which include, but are not limited to:

### (i) SEQ ID NO: 146;

### (ii) SEQ ID NO: 130;

(iii) SEQ ID NO: 113;

### (iv) SEQ ID NO: 96;

### (v) SEQ ID NO: 90;

### (vi) SEQ ID NO: 73;

(vii) SEQ ID NO: 58;
(viii) SEQ ID NO: 53;

### (ix) SEQ ID NO: 35; and

### (x) SEQ ID NO: 17.

In another example, the light chain variable region of the antigen specific binding domain of the first aspect comprises a sequence which include, but are not limited to:

### (i) SEQ ID NO: 138;

### (ii) SEQ ID NO: 122;

(iii) SEQ ID NO: 105

### (iv) SEQ ID NO: 93;

### (v) SEQ ID NO: 81

### (vi) SEQ ID NO: 65;

(vii) SEQ ID NO: 8;
(viii) SEQ ID NO: 44;

### (ix) SEQ ID NO: 26; and

### (x) SEQ ID NO: 8.

In another example, the antigen specific binding domain of the first aspect comprises a VH/VL pair which include, but are not limited to any one of:

### (i) SEQ ID NO: 146 and SEQ ID NO: 138;

### (ii) SEQ ID NO: 130 and SEQ ID NO: 122;

(iii) SEQ ID NO: 113 and SEQ ID NO: 105;

### (iv) SEQ ID NO: 96 and SEQ ID NO: 93;

### (v) SEQ ID NO: 90 and SEQ ID NO: 81;

### (vi) SEQ ID NO: 73 and SEQ ID NO: 65;

(vii) SEQ ID NO: 58 and SEQ ID NO: 8;
(viii) SEQ ID NO: 53 and SEQ ID NO: 44

### (ix) SEQ ID NO: 35 and SEQ ID NO: 26; and

### (x) SEQ ID NO: 17 and SEQ ID NO: 8.

In a second aspect, the present disclosure provides a chimeric antigen receptor comprising the antigen specific binding domain of the first aspect.

As used herein, the term “chimeric antigen receptor” or “CAR” in the short form refers to molecules that combine antibody-based specificity for an antigen with cell receptor-activating intracellular domains with specific cellular immune activity. In some examples, a CAR comprises at least an extracellular antigen binding domain, a transmembrane domain, and a cytoplasmic signaling domain (also referred to herein as “an intracellular signaling domain”) comprising a functional signaling domain derived from a stimulatory molecule and/or costimulatory molecule. In some examples, the intracellular signaling domain is CD3-zeta, for from any of the intracellular signaling domains as described for example in WO2013/040557. In some examples, the chimeric antigen receptor comprises a co-stimulator domain independently selected from CD28, 4-1BB or OX40, and combinations thereof, such as CD28 and 4-1BB or CD28 and OX40.

In some examples, the chimeric antigen receptor may combine the antigen specific binding domain as described herein, with a cell receptor-activating intracellular domain (e.g. T cell receptor-activating intracellular domain) with specific cellular immune activity. The chimeric antigen receptor would then allow an immune cell to achieve MHC-independent primary activation through a single chain Fv (scFv) antigen-specific extracellular region fused to intracellular domains that provide T cell activation and co-stimulatory signals.

Therefore, in one example, the present disclosure provides a chimeric antigen receptor comprising an extracellular antigen binding domain, wherein the extracellular antigen binding domain comprises the antigen specific binding domain disclosed herein. In this example, the chimeric antigen receptor selectively binds with RON.

Therefore, in a third aspect, the present disclosure provides a cell expressing the chimeric antigen receptor as escribed above. In some examples, cell is selected from a T cell (such as a cytotoxic T cell), a Natural Killer (NK) cell and a Natural Killer T (NKT) cell.

Also disclosed herein are antibodies which bind specifically to RON, or the extracellular portion of the human RON protein. Therefore, in a fourth aspect, the present disclosure provides an antibody molecule comprising the antigen specific binding domain according to the first aspect.

The term “antibody molecule” as used herein can refer to an antibody or a fragment thereof. In one example, the fragment is an antigen binding fragment. The antibody may have full length heavy and light chains and which has an immunoglobulin Fc region. Variable regions of the heavy and ligjht chains generally exhibit the same overall structure, comprising relatively conserved framework regions (FR) joined by three hypervariable regions, more often called “complementarity determining regions” or CDRs. The CDRs from the two chains of each heavy chain/light chain pair mentioned above typically are aligned by the framework regions to form a structure that binds specifically with a specific epitope on the target protein (e.g., RON). From N-terminal to C-terminal, naturally-occurring light and heavy chain variable regions both typically conform with the following order of these elements: FR1, CDR1, FR2, CDR2, FR3, CDR3 and FR4. A numbering system has been devised for assigning numbers to amino acids that occupy positions in each of these domains. This numbering system is defined in Kabat Sequences of Proteins of Immunological Interest (1987 and 1991, NIH, Bethesda, Md.), or Chothia & Lesk, 1987, J. Mol. Biol. 196:901-917; Chothia et al., 1989, Nature 342:878-883.

The antigen binding fragment includes, but is not limited to Fab, modified Fab, Fab′, modified Fab′, F(ab′)2, Fv, Fab-Fv, Fab-dsFv, scFv, and a minibody. As used herein, a minibody refers to a single chain polypeptide that comprises a secretion signal, a variable heavy chain fragment (VH), a variable light chain fragment (VL) and a constant chain fragment (CH3).

A “binding fragment” as employed herein refers to an antibody fragment capable of binding a target peptide or antigen with sufficient specificity to characterise the fragment as specific for the peptide or antigen. “Specific” as employed herein refers to an antibody molecule that only recognises the antigen to which it is specific or an antibody molecule that has significantly higher binding affinity for the antigen to which it is specific compared to the binding affinity for antigens to which it is non-specific, for example 5, 6, 7, 8, 9, 10 or more times higher binding affinity. The term “Fab fragment” as used herein refers to an antibody fragment comprising a light chain fragment comprising a V_{L} (variable light) domain and a constant domain of a light chain (C_{L}), and a V_{H} (variable heavy) domain and a first constant domain (CH₁) of a heavy chain. Fv refers to two variable domains, for example co-operative variable domains, such as a cognate pair or affinity matured variable domains, such as a V_{H} and V_{L} pair. Co-operative variable domains as employed herein are variable domains that complement each other and/or both contribute to antigen binding to render the Fv (V_{H}/V_{L} pair) specific for the antigen in question. Binding domain as employed herein refers to two co-operative variable regions, such as a VH and VL each comprising 3 CDRs, wherein the binding domain is specific to a particular (target) antigen.

In some examples, the antibody molecule is a multispecific antibody. In some examples, the multispecific antibody a bi-specific antibody. In some examples, the multispecific antibody is a bi, tri or tetra-specific antibody. The multispecific antibodies disclosed herein also include other antibody fragments/fusions such as Bis-scFv, diabodies, triabodies, tetrabodies and other epitope-binding fragments.

The antibody molecule as employed herein may comprise multiple specificities e.g. bispecific. Bispecific and multispecific antibody variants are especially relevant as the role of the therapeutic molecules of the present disclosure in one example as the therapeutic objective is to inhibit two independent target proteins, namely RON receptor tyrosine kinase and for example second therapeutic target, such as a protein in the PD-1 pathway (in particular PD-1 or PD-L1). Therefore, in one example, the antibody molecule comprises a first antigen binding domain specifically binding to RON, and a second antigen binding domain specifically binding to another protein target. In a specific example, the protein target is an immune checkpoint point, for example PD-1, PD-L1 or CTLA-4.

In some examples, the antibody molecule is a bi-specific antibody targeting two distinct epitopes of RON. In this example, the antibody molecule comprises a first antigen binding domain specifically binding to a first RON epitope, and a second antigen binding domain specifically binding to a second RON epitope which is different from the first RON epitope. In some examples, each of the first and second antigen binding domains is an antigen specific binding domain according to the first aspect of the disclosure, wherein the first and second antigen binding domains are different.

In the context of the antibodies specifically referred to herein it will be clear to the skilled person that the disclosure includes use of the variable domains, in particular as a pair from the said antibody in any antibody or fragment format, including a multispecific antibody. In one embodiment 6 CDRs are employed from an antibody disclosed herein in combination with an alternative framework, such as a human framework.

“Multispecific” as employed herein refers to the ability to specifically bind at least two distinct epitopes, which can be on the same or different antigens. Multi-valent antibodies may comprise multiple specificities e.g. bispecific or may be monospecific.

The antibody molecules of the present disclosure have high affinity towards RON. Binding affinity (affinity) may be measured by a number of standard assays, for example surface plasmon resonance, such as BIAcore or Kinetic Exclusion Assay (such as KinExA). In one example, the antibody molecule binds with RON with a Kd of 15 nM or less, a Kd of 10 nM or less, a Kd of 5 nM or less, a Kd of 1 nM or less, a Kd of 0.6 nM or less, a Kd of 0.3 nM or less, a Kd of 0.15 nM or less, a Kd of 0.1 nM or less, a Kd of 0.03 nM or less, or a Kd of 0.029 nM or less, or a Kd of 0.02 nM or less, or a Kd of 0.01 nM or less. In one example, the Kd is determined using a kinetic exclusion assay (such as KinExA).

The affinity of the original antibody may be increased by employing an affinity maturation protocols including mutating the CDRs, chain shuffling, use of mutator strains of E. coli, DNA shuffling, phage display and sexual PCR. Increased affinity as employed herein in this context refers to an improvement over the starting molecule.

In another example, the antibody molecule as disclosed herein inhibits macrophage stimulating protein (MSP) induced pERK activation with an IC₅₀ of 100 nM or less, or with an IC50 of 70 nM or less, or with an IC₅₀ of 31 nM or less, or with an IC₅₀ of 20 nM or less, or with an IC₅₀ of 14.8 nM or less, or with an IC₅₀ of 13.9 nM or less, or with an IC₅₀ of 13.3 nM or less, or with an IC₅₀ of 8 nM or less, or with an IC₅₀ of 5 nM or less, or with an IC₅₀ of 3 nM or less, or with an IC₅₀ of 2.8 nM or less. In one example, the p-ERK level analyses is performed using the AlphaLISA SureFire Ultra p-ERK1/2 (Thr202/Tyr204) Assay Kit (Perkin Elmer) as per manufacturer's protocol.

In some examples, the antibody molecules as disclosed herein exhibits antibody dependent cellular cytotoxicity against cancer cells. In one example the antibody molecules as disclosed herein exhibits antibody dependent cellular cytotoxicity against breast cancer cell, or specifically T47D breast cancer cells. In one example, the antibody molecules as disclosed herein exhibits antibody dependent cellular cytotoxicity against cancer cells in vitro with an EC₅₀ of 500 pM or less, or with an EC50 of 400 pM or less, or with an EC₅₀ of 200 pM or less, or with an EC₅₀ of 108 pM or less, or with an EC₅₀ of 22 pM or less, or with an EC₅₀ of 13 pM or less, or with an EC₅₀ of 3 pM or less.

In one example, an antibody molecule according to the present disclosure is chimeric. In one example, the antibody molecule disclosed herein is a chimeric antibody, or a mouse/human chimeric antibody. In one example, wherein the antibody molecule is a chimeric antibody, the chimeric antibody comprises a Fragment crystallisable region (Fc) of human immunoglobulin or a backbone thereof. In one example, the chimeric antibody comprises a Fragment antigen binding (Fab) of a mouse antibody.

In one example, an antibody molecule according to the present disclosure is humanized, or specifically a humanized antibody.

In one example, an antibody molecule according to the present disclosure is a monoclonal antibody.

In one example the antibody molecule of the present disclosure comprises an amino acid sequence at least 95% identical to a sequence disclosed herein. This embodiment also extends to sequences 96, 97, 98 or 99% to a given sequence disclosed herein.

For example, the present disclosure provides an antibody molecule comprising:

(i) a heavy chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 146 and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 138;
(ii) a heavy chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 130 and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 122;
(iii) a heavy chain variable region comprising the sequence of SEQ ID NO: 113 and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 105;
(iv) a heavy chain variable region comprising the sequence of SEQ ID NO: 96 and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 93;
(v) a heavy chain variable region comprising the sequence of SEQ ID NO: 90 and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 81;
(vi) a heavy chain variable region comprising the sequence of SEQ ID NO: 73 and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 65;
(vii) a heavy chain variable region comprising an amino acid sequence having a sequence identity of at least 85%, or at least 90%, or at least 95%, or at least 96%, or at least 97%, or at least 98%, or at least 99%, or at least 99.95%, or 100% of ID NO: 58 and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 8;
(viii) a heavy chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 53 and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 44
(ix) a heavy chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 35 and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 26; or
(x) a heavy chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 17 and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 8.

In another example, the present disclosure provides an antibody molecule comprising:

(i) a heavy chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 146 in regions excluding the CDRs, and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 138 in regions excluding the CDRs;
(ii) a heavy chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 130 in regions excluding the CDRs, and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 122 in regions excluding the CDR;
(iii) a heavy chain variable region comprising an amino acid sequence having a sequence identity of SEQ ID NO: 113 in regions excluding the CDRs, and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 105 in regions excluding the CDR;
(iv) a heavy chain variable region comprising an amino acid sequence having a sequence identity of SEQ ID NO: 96 in regions excluding the CDRs, and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 93 in regions excluding the CDR;
(v) a heavy chain variable region comprising an amino acid sequence having a sequence identity of SEQ ID NO: 90 in regions excluding the CDRs, and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 81 in regions excluding the CDR;
(vi) a heavy chain variable region comprising an amino acid sequence having a sequence identity of SEQ ID NO: 73 in regions excluding the CDRs, and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 65 in regions excluding the CDR;
(vii) a heavy chain variable region comprising an amino acid sequence having a sequence identity of at least 85%, or at least 90%, or at least 95%, or at least 96%, or at least 97%, or at least 98%, or at least 99%, or at least 99.95%, or 100% of ID NO: 58 in regions excluding the CDRs, and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 8 in regions excluding the CDR;
(viii) a heavy chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 53 in regions excluding the CDRs, and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 44 in regions excluding the CDRs
(ix) a heavy chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 35 in regions excluding the CDRs, and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 26 in regions excluding the CDRs; or
(x) a heavy chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 17 in regions excluding the CDRs, and a light chain variable region comprising an amino acid sequence having a sequence identity of at least 85%, or at least 90%, or at least 95%, or at least 96%, or at least 97%, or at least 98%, or at least 99%, or at least 99.95%, or 100% of SEQ ID NO: 8 in regions excluding the CDRs.

The methods for creating and manufacturing these antibodies and antibody fragments are well known in the art. Antibodies for use in the present disclosure may be obtained using any suitable method known in the art. The polypeptide/protein including: fusion proteins, for example polypeptide-Fc fusions proteins; or cells, recombinantly or naturally, expressing the polypeptide (as activated T cells), can be used to, for example immunise a host and produce antibodies which specifically recognise the target polypeptide/protein. The polypeptide may be the full length polypeptide or a biologically active fragment or derivative thereof.

Polypeptides may be prepared by processes well known in the art from genetically engineered host cells comprising expression systems or they may be recovered from natural biological sources. In the present application, the term “polypeptides” includes peptides, polypeptides and proteins. These are used interchangeably unless otherwise specified. The antigen polypeptide may in some instances be part of a larger protein such as a fusion protein for example fused to an affinity tag.

Antibodies generated against the antigen polypeptide may be obtained, where immunisation of an animal is necessary, by administering the polypeptides to an animal, preferably a non-human animal, using well-known and routine protocols. Many warm-blooded animals, such as rabbits, mice, rats, sheep, cows, camels or pigs may be immunized. However, mice, rabbits, pigs and rats are generally most suitable.

Monoclonal antibodies may be prepared by any method known in the art such as the hybridoma technique, the trioma technique, the human B-cell hybridoma technique and the EBV-hybridoma technique.

Antibodies for use in the disclosure may also be generated using single lymphocyte antibody methods by cloning and expressing immunoglobulin variable region cDNAs generated from single lymphocytes selected for the production of specific antibodies.

Humanized antibodies (which include CDR-grafted antibodies) are antibody molecules having one or more complementarity determining regions (CDRs) from a non-human species and a framework region from a human immunoglobulin molecule. It will be appreciated that it may only be necessary to transfer the specificity determining residues of the CDRs rather than the entire CDR. Humanised antibodies may optionally further comprise one or more framework residues derived from the non-human species from which the CDRs were derived.

As used herein, the term ‘humanized antibody molecule’ refers to an antibody molecule wherein the heavy and/or light chain contains one or more CDRs (including, if desired, one or more modified CDRs) from a donor antibody (e.g. a murine monoclonal antibody) grafted into a heavy and/or light chain variable region framework of an acceptor antibody (e.g. a human antibody). In one embodiment rather than the entire CDR being transferred, only one or more of the specificity determining residues from any one of the CDRs described herein above are transferred to the human antibody framework. In one embodiment only the specificity determining residues from one or more of the CDRs described herein above are transferred to the human antibody framework. In another embodiment only the specificity determining residues from each of the CDRs described herein above are transferred to the human antibody framework.

When the CDRs or specificity determining residues are grafted, any appropriate acceptor variable region framework sequence may be used having regard to the class/type of the donor antibody from which the CDRs are derived, including mouse, primate and human framework regions. Suitably, the humanized antibody according to the present invention has a variable domain comprising human acceptor framework regions as well as one or more of the CDRs provided herein.

In some examples, the present disclosure provides an antibody molecule comprising a light chain variable region (VL) and a heavy chain variable region (VH), wherein the heavy chain variable region (VH) comprises Complementarity Determining Regions (CDRs) CDRH1, CDRH2, CDRH3, and the light chain variable region comprises CDRs CDRL1, CDRL2, and CDRL3; wherein:

### (a) the CDRL1 is SEQ ID NO: 132, the CDRL2 is SEQ ID NO: 133, the CDRL3 is SEQ ID NO: 134, the CDRH1 is SEQ ID NO: 140, the CRDH2 is SEQ ID NO: 141, and the CRDH3 is SEQ ID NO: 142;

### (b) the CDRL1 is SEQ ID NO: 1, the CDRL2 is SEQ ID NO: 2, the CDRL3 is SEQ ID NO: 3, the CDRH1 is SEQ ID NO: 10, the CRDH2 is SEQ ID NO: 11, and the CRDH3 is SEQ ID NO: 12;

### (c) the CDRL1 is SEQ ID NO: 19, the CDRL2 is SEQ ID NO: 20, the CDRL3 is SEQ ID NO: 21 the CDRH1 is SEQ ID NO: 28, the CRDH2 is SEQ ID NO: 29, the CRDH3 is SEQ ID NO: 30;

### (d) the CDRL1 is SEQ ID NO: 37, the CDRL2 is SEQ ID NO: 38, the CDRL3 is SEQ ID NO: 39, the CDRH1 is SEQ ID NO: 46, the CRDH2 is SEQ ID NO: 47, and the CRDH3 is SEQ ID NO: 48;

### (e) the CDRL1 is SEQ ID NO: 60, the CDRL2 is SEQ ID NO: 20, the CDRL3 is SEQ ID NO: 61, the CDRH1 is SEQ ID NO: 67, the CRDH2 is SEQ ID NO: 68, and the CRDH3 is SEQ ID NO: 69;

### (f) the CDRL1 is SEQ ID NO: 75, the CDRL2 is SEQ ID NO: 76, the CDRL3 is SEQ ID NO: 77, the CDRH1 is SEQ ID NO: 83, the CRDH2 is SEQ ID NO: 84, and the CRDH3 is SEQ ID NO: 85;

### (g) the CDRL1 is SEQ ID NO: 98, the CDRL2 is SEQ ID NO: 99, the CDRL3 is SEQ ID NO: 100, the CDRH1 is SEQ ID NO: 107, the CRDH2 is SEQ ID NO: 108, and the CRDH3 is SEQ ID NO: 109; or

### (h) the CDRL1 is SEQ ID NO: 115, the CDRL2 is SEQ ID NO: 116, the CDRL3 is SEQ ID NO: 117, the CDRH1 is SEQ ID NO: 124, the CRDH2 is SEQ ID NO: 125, and the CRDH3 is SEQ ID NO: 126.

Also disclosed herein is an antibody molecule which cross-blocks or binds the same epitope as an antibody molecule comprising a VH of SEQ ID NO: 17, SEQ ID NO: 35, SEQ ID NO: 53, SEQ ID NO: 51, SEQ ID NO: 63, SEQ ID NO: 79, SEQ ID NO: 95, SEQ ID NO: 106, SEQ ID NO: 121, SEQ ID NO: 130, SEQ ID NO: 133 or 142.

Also disclosed herein is an antibody molecule which cross-blocks or binds the same epitope as an antibody molecule comprising a VH/VL pair selected from SEQ ID NO: 8 and 25, SEQ ID NO: 16 and 25, SEQ ID NO: 34 and 43, SEQ ID NO: 51 and 54, SEQ ID NO: 63 and 71, SEQ ID NO: 79 and 87, SEQ ID NO: 95 and 98, SEQ ID NO: 106 and 114, SEQ ID NO: 121 and 130 or SEQ ID NO: 142 and 150.

In another example, also disclosed herein is an antibody which cross-blocks or binds the same epitope as the antibody molecule as disclosed herein. The term “cross-block,” as used herein refers to the ability of an antibody to interfere with the binding of other antibodies or binding fragments to the antigen (e.g. the human RON protein or the extracellular domain thereof) or the epitope. The extent to which an antibody or binding fragment is able to interfere with the binding of another to the RON protein, and therefore whether it can be said to cross-block, can be determined using competition binding assays. In some embodiments, a cross-blocking antibody or binding fragment thereof reduces human RON binding of an antibody as disclosed herein between about 40% and 100%, such as about 60% and about 100%, specifically between about 70% and 100%, and more specifically between about 80% and 100%. A particularly suitable quantitative assay for detecting cross-blocking uses a Biacore machine which measures the extent of interactions using surface plasmon resonance technology. Another suitable quantitative cross-blocking assay uses a FACS-based approach to measure competition between antibodies in terms of their binding to the human RON protein.

In one example, an antibody molecule according to the present disclosure may be conjugated to at least one payload. It will be appreciated that the payload may comprise a single molecule or two or more such molecules so linked as to form a single moiety that can be attached to the antibodies of the present invention. Where it is desired to obtain an antibody fragment linked to a molecule, this may be prepared by standard chemical or recombinant DNA procedures in which the antibody fragment is linked either directly or via a coupling agent to the effector molecule. Techniques for conjugating such effector molecules to antibodies are well known in the art. Alternatively, where the molecule is a protein or polypeptide the linkage may be achieved using recombinant DNA procedures.

The term payloads as used herein includes, for example, biologically active proteins, for example enzymes, other antibody or antibody fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

In a fifth aspect, the present disclosure provides a polynucleotide encoding the antigen specific binding domain of the first aspect, the chimeric antigen receptor of the second aspect, or the antibody molecule of the fourth aspect.

### Pharmaceutical Composition

In a sixth aspect, the present disclosure provides a pharmaceutical formulation or composition according to the invention comprises an antibody molecule according to the present disclosure and a pharmaceutically acceptable excipient, diluent and/or carrier.

Such carriers enable the pharmaceutical compositions to be formulated as tablets, pills, dragees, capsules, liquids, gels, syrups, slurries and suspensions, for ingestion by the patient.

Pharmaceutically acceptable carriers in therapeutic compositions may additionally contain liquids such as water, saline, glycerol and ethanol. Additionally, auxiliary substances, such as wetting or emulsifying agents or pH buffering substances, may be present in such compositions.

The pharmaceutical compositions of this invention may be administered by any number of routes including, but not limited to, oral, intravenous, intramuscular, intra-arterial, intramedullary, intrathecal, intraventricular, transdermal, transcutaneous, subcutaneous, intraperitoneal, intranasal, enteral, topical, sublingual, intravaginal or rectal routes. Hyposprays may also be used to administer the pharmaceutical compositions of the invention. Typically, the therapeutic compositions may be prepared as injectables, either as liquid solutions or suspensions. Solid forms suitable for solution in, or suspension in, liquid vehicles prior to injection may also be prepared.

Suitable forms for administration include forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilising and/or dispersing agents. Alternatively, the antibody molecule may be in dry form (such as lyophilised), for reconstitution before use with an appropriate sterile liquid, such as glycose, saline, water for injection or a combination of two or more of the same.

Thus in one example the formulation is provided for parenteral administration, in particular for intravenous or subcutaneous injection.

Once formulated, the compositions of the invention can be administered directly to the subject. The subjects to be treated can be animals. However, in one or more embodiments the compositions are adapted for administration to human subjects.

Suitably in formulations according to the present disclosure, the pH of the final formulation is not similar to the value of the isoelectric point of the antibody or fragment, for example if the pH of the formulation is 7 then a pI of from 8-9 or above may be appropriate. Whilst not wishing to be bound by theory it is thought that this may ultimately provide a final formulation with improved stability, for example the antibody or fragment remains in solution.

In one example the pH of a liquid formulation according to the present disclosure is in range pH 5.5 to 8, such as pH 6, 6.5, 7, 7.1, 7.2, 7.3, 7.4, 7.5, 7.6, 7.7, 7.8 or 7.9.

In one example the formulation is provide is isotonic or is isotonic after constitution.

In one example the composition or formulation of the present disclosure comprises 1-200 mg/mL of an antibody molecule according to the present disclosure, for example 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68. 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198 or 199 mg/ml.

### Treatment

In a seventh aspect, the present disclosure provides the antigen specific binding domain of the first aspect, cell of the third aspect, the antibody molecule of the fourth aspect, the polynucleotide of the fifth aspect, or the pharmaceutical composition of the sixth aspect for use in therapy, for example for use in treating cancer.

In an eighth aspect, the present disclosure refers to the use of the antigen specific binding domain of the first aspect, cell of the third aspect, the antibody molecule of the fourth aspect, the polynucleotide of the fifth aspect, or the pharmaceutical composition of the sixth aspect in the manufacture of a medicament for the treatment of cancer.

In a ninth aspect, the present disclosure refers to a method of treating a patient for cancer comprising administering a therapeutically effective amount of the antigen specific binding domain of the first aspect, cell of the third aspect, the antibody molecule of the fourth aspect, the polynucleotide of the fifth aspect, or the pharmaceutical composition of the sixth aspect.

In one example, the cancer to be treated is selected from liver cancer (such as hepatocellular carcinoma), biliary duct cancer, breast cancer (such as non ER+ breast cancer), prostate cancer, colorectal cancer, ovarian cancer, endometrial cancer, cervical cancer, lung cancer, gastric cancer, oesophageal cancer, pancreatic, bone cancer, bladder cancer, head and neck cancer, thyroid cancer, skin cancer, renal cancer, and oesophagus cancer and combinations thereof, for example gastric cancer.

In one example, the cancer is selected from selected from the group comprising hepatocellular carcinoma, biliary duct cancer, breast cancer, prostate cancer, colorectal cancer, ovarian cancer, lung cancer, gastric cancer, pancreatic and oesophagus cancer.

In one example, the biliary duct cancer is in a location selected from intrahepatic bile ducts, left hepatic duct, right hepatic duct, common hepatic duct, cystic duct, common bile duct, Ampulla of Vater and combinations thereof.

In one example, the biliary duct cancer is in an intrahepatic bile duct. In one embodiment the biliary duct cancer is in a left hepatic duct. In one embodiment the biliary duct cancer is in a right hepatic duct. In one embodiment the biliary duct cancer is in a common hepatic duct. In one embodiment the biliary duct cancer is in a cystic duct. In one example the biliary duct cancer is in a common bile duct. In one example the biliary duct cancer is in an Ampulla of Vater.

In one example the epithelial cancer is a carcinoma.

In one example the cancer is tumour, for example a solid tumour, a liquid tumour or a combination of the same.

In one example the treatment according to the disclosure is adjuvant therapy, for example after surgery.

In one example the therapy according to the disclosure is neoadjuvant treatment, for example to shrink a tumour before surgery.

In one example the tumour is a solid tumour. In one example the cancer is a primary cancer, secondary cancer, metastasis or combination thereof. In one example the treatment according to the present disclosure is suitable for the treatment of secondary tumours. In one example the cancer is metastatic cancer. In one example the treatment according to the present disclosure is suitable for the treatment of primary cancer and metastases. In one example the treatment according to the present disclosure is suitable for the treatment of secondary cancer and metastases. In one example the treatment according to the present disclosure is suitable for the treatment of primary cancer, secondary cancer and metastases.

In one example the treatment according to the present disclosure is suitable for the treatment of cancerous cells in a lymph node, for a cancer of the present disclosure.

In one example the cancer is RON positive. In one example the cancer is Met positive. In one example the cancer is RON positive and Met positive.

In one example the cancer is refractory or resistant to one or more available cancer treatments.

In one example the therapy of the present disclosure is first line therapy. In one example the therapy according to the present disclosure is second line or subsequent line therapy.

In one example the antibody molecule according to the present disclosure is employed in a combination therapy.

In one example the combination therapy comprises a checkpoint inhibitor, such as a CTLA4 inhibitor, a PD-1 inhibitor or a PD-L1 inhibitor, in particular an antibody or binding fragment thereof.

In one example the combination therapy of the present disclosure comprises or further comprises a chemotherapeutic agent.

In one example the combination therapy comprises a HER inhibitor, for example herceptin or the pan-HER inhibitor varlitinib [(R)—N4-[3-Chloro-4-(thiazol-2-ylmethoxy)-phenyl]-N6-(4-methyl-4,5,-dihydro-oxazol-2-yl)-quinazoline-4,6-diamine (Varlitinib Example 52 disclosed in WO2005/016346)] for example administered once or twice daily at a dose in the range 100 mg to 500 mg, such as 200 mg, 300 mg or 400 mg.

Chemotherapeutic agent and chemotherapy or cytotoxic agent are employed interchangeably herein unless the context indicates otherwise.

Chemotherapy as employed herein is intended to refer to specific antineoplastic chemical agents or drugs that are “selectively” destructive to malignant cells and tissues, for example alkylating agents, antimetabolites including thymidylate synthase inhibitors, anthracyclines, anti-microtubule agents including plant alkaloids, taxanes, topoisomerase inhibitors, parp inhibitors and other antitumour agents. Selectively in this context is used loosely because of course many of these agents have serious side effects.

The preferred dose may be chosen by the practitioner, based on the nature of the cancer being treated.

Examples of alkylating agents, which may be employed in the method of the present disclosure include an alkylating agent, nitrogen mustards, nitrosoureas, tetrazines, aziridines, platins and derivatives, and non-classical alkylating agents.

Examples of platinum containing chemotherapeutic agents (also referred to as platins), include cisplatin, carboplatin, oxaliplatin, satraplatin, picoplatin, nedaplatin, triplatin and lipoplatin (a liposomal version of cisplatin), in particular cisplatin, carboplatin and oxaliplatin.

The dose for cisplatin ranges from about 20 to about 270 mg/m² depending on the exact cancer. Often the dose is in the range about 70 to about 100 mg/m².

Nitrogen mustards include mechlorethamine, cyclophosphamide, melphalan, chlorambucil, ifosfamide and busulfan.

Nitrosoureas include N-Nitroso-N-methylurea (MNU), carmustine (BCNU), lomustine (CCNU) and semustine (MeCCNU), fotemustine and streptozotocin. Tetrazines include dacarbazine, mitozolomide and temozolomide.

Aziridines include thiotepa, mytomycin and diaziquone (AZQ).

Examples of antimetabolites, which may be employed in the method of the present disclosure, include anti-folates (for example methotrexate and pemetrexed), purine analogues (for example thiopurines, such as azathiopurine, mercaptopurine, thiopurine, fludarabine (including the phosphate form), pentostatin and cladribine), pyrimidine analogues (for example fluoropyrimidines, such as 5-fluorouracil (5-FU) and prodrugs thereof such as capecitabine [Xeloda®]), floxuridine, gemcitabine, cytarabine, decitabine, raltitrexed (tomudex) hydrochloride, cladribine and 6-azauracil.

Examples of anthracyclines, which may be employed in the method of the present disclosure, include daunorubicin (Daunomycin), daunorubicin (liposomal), doxorubicin (Adriamycin), doxorubicin (liposomal), epirubicin, idarubicin, valrubicin currently are used only to treat bladder cancer and mitoxantrone an anthracycline analog, in particular doxorubicin.

Examples of anti-microtubule agents, which may be employed in the method of the present disclosure, include include vinca alkaloids and taxanes.

Vinca alkaloids include completely natural chemicals for example vincristine and vinblastine and also semi-synthetic vinca alkaloids, for example vinorelbine, vindesine, and vinflunine.

Taxanes include paclitaxel, docetaxel, abraxane, carbazitaxel and derivatives of thereof. Derivatives of taxanes as employed herein includes reformulations of taxanes like taxol, for example in a micelluar formulaitons, derivatives also include chemical derivatives wherein synthetic chemistry is employed to modify a starting material which is a taxane.

Topoisomerase inhibitors, which may be employed in a method of the present disclosure include type I topoisomerase inhibitors, type II topoisomerase inhibitors and type II topoisomerase poisons. Type I inhibitors include topotecan, irinotecan, indotecan and indimitecan. Type II inhibitors include genistein and ICRF 193 which has the following structure:

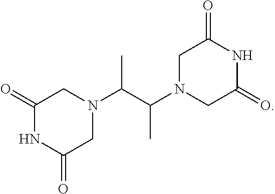 [SMILES: CC(C(C)N1CC(=O)NC(=O)C1)N1CC(=O)NC(=O)C1]

Type II poisons include amsacrine, etoposide, etoposide phosphate, teniposide and doxorubicin and fluoroquinolones.

In one example the chemotherapeutic is a PARP inhibitor.

In one example a combination of chemotherapeutic agents employed is, for example a platin and 5-FU or a prodrug thereof, for example cisplatin or oxaplatin and capecitabine or gemcitabine, such as FOLFOX.

In one example the chemotherapy comprises a combination of chemotherapy agents, in particular cytotoxic chemotherapeutic agents. In one example the chemotherapy combination comprises a platin, such as cisplatin and fluorouracil or capecitabine. In one example the chemotherapy combination is capecitabine and oxaliplatin (XELOX). In one example the chemotherapy is a combination of folinic acid and 5-FU, optionally in combination with oxaliplatin (FOLFOX). In one example the chemotherapy is a combination of folinic acid, 5-FU and irinotecan (FOLFIRI), optionally in combination with oxaliplatin (FOLFIRINOX). The regimen, for example includes: irinotecan (180 mg/m² IV over 90 minutes) concurrently with folinic acid (400 mg/m² [or 2×250 mg/m²] IV over 120 minutes); followed by fluorouracil (400-500 mg/m² IV bolus) then fluorouracil (2400-3000 mg/m² intravenous infusion over 46 hours). This cycle is typically repeated every two weeks. The dosages shown above may vary from cycle to cycle. In one example the chemotherapy combination employs a microtubule inhibitor, for example vincristine sulphate, epothilone A, N-[2-[(4-Hydroxyphenyl)amino]-3-pyridinyl]-4-methoxybenzenesulfonamide (ABT-751), a taxol derived chemotherapeutic agent, for example paclitaxel, abraxane, or docetaxel or a combination thereof. In one example the chemotherapy combination employs an mTor inhibitor. Examples of mTor inhibitors include: everolimus (RAD001), WYE-354, KU-0063794, papamycin (Sirolimus), Temsirolimus, Deforolimus (MK-8669), AZD8055 and BEZ235 (NVP-BEZ235). In one example the chemotherapy combination employs a MEK inhibitor. Examples of MEK inhibitors include: AS703026, CI-1040 (PD184352), AZD6244 (Selumetinib), PD318088, PD0325901, AZD8330, PD98059, U0126-EtOH, BIX 02189 or BIX 02188. In one example the chemotherapy combination employs an AKT inhibitor. Examples of AKT inhibitors include: MK-2206 and AT7867. In one example the combination employs an aurora kinase inhibitor. Examples of aurora kinase inhibitors include: Aurora A Inhibitor I, VX-680, AZD1152-HQPA (Barasertib), SNS-314 Mesylate, PHA-680632, ZM-447439, CCT129202 and Hesperadin. In one example the chemotherapy combination employs a p38 inhibitor, such as N-[4-({4-[3-(3-tert-Butyl-1-p-tolyl-1H-pyrazol-5-yl)ureido]naphthalen-1-yloxy}methyl)pyridin-2-yl]-2-methoxyacetamide. In one example the combination employs a Bcl-2 inhibitor. Examples of Bcl-2 inhibitors include: obatoclax mesylate, ABT-737, ABT-263 (navitoclax) and TW-37. In one example the chemotherapy combination comprises an antimetabolite such as capecitabine (xeloda), fludarabine phosphate, fludarabine (fludara), decitabine, raltitrexed (tomudex), gemcitabine hydrochloride and/or cladribine. In one example the chemotherapy combination comprises ganciclovir, which may assist in controlling immune responses and/or tumour vasculation.

“Administering a combination therapy” as employed herein does not require the therapies employed in the combination to be administered at the same time. Combination therapy as employed herein refers to two or more modes of therapy being employing over the same treatment period, i.e. the opposite of sequential therapy. Two or more modes of therapy as employed herein refers to at least two therapies which have different modes of action and/or different activities and/or different routes of administration.

Terms such as “treating” or “treatment” or “to treat” as employed herein refers to therapeutic measures that: cure, slow down, ameliorate symptoms of, and/or halt progression of a diagnosed pathologic condition or disorder; or prophylactic or preventative measures that prevent and/or slow the development of a targeted pathologic condition or disorder. Thus, those in need of treatment include those already with the disorder; those prone to have the disorder; and those in whom the disorder is to be prevented and those in whom reoccurrence of the disorder needs to be prevented (e.g. those in remission). In certain aspects, a subject is successfully “treated” for a disease or condition, for example cancer if the patient shows, e.g. total, partial, or transient remission of the disease or condition.

A therapeutically effective amount as employed herein refers to an amount suitable to elicit the requisite therapeutic effect. In the combination therapy of the present disclosure the RON inhibitor may be employed at a dose which is the same or lower than the monotherapy dose with said inhibitor. In one example the dose of the PD-1 pathway inhibitor employed is the same or lower than the monotherapy dose with said inhibitor. In one example the dose on the RON inhibitor is the same as the dose employed in monotherapy and the dose of the PD-1 pathway inhibitor is the same as the dose employed in monotherapy. In one example the dose on the RON inhibitor is lower than the dose employed in monotherapy and the dose of the PD-1 pathway inhibitor is the same as the dose employed in monotherapy. In one embodiment the dose on the RON inhibitor is the same as the dose employed in monotherapy and the dose of the PD-1 pathway inhibitor is lower than the dose employed in monotherapy. In one embodiment the dose on the RON inhibitor is lower the dose employed in monotherapy and the dose of the PD-1 pathway inhibitor is lower than the dose employed in monotherapy. A suitable dose can be established by those skilled in the art.

### Non-Limiting Examples of Cancers

Common Epithelial Tumors—Epithelial ovarian tumors develop from the cells that cover the outer surface of the ovary. Most epithelial ovarian tumors are benign (noncancerous). There are several types of benign epithelial tumors, including serous adenomas, mucinous adenomas, and Brenner tumors. Cancerous epithelial tumors are carcinomas—meaning they begin in the tissue that lines the ovaries. These are the most common and most dangerous of all types of ovarian cancers. Unfortunately, almost 70 percent of women with the common epithelial ovarian cancer are not diagnosed until the disease is advanced in stage.

There are some ovarian epithelial tumors whose appearance under the microscope does not clearly identify them as cancerous. These are called borderline tumors or tumors of low malignant potential (LMP tumors). The method of the present disclosure includes treatment of the latter.

Germ Cell Tumors—Ovarian germ cell tumors develop from the cells that produce the ova or eggs. Most germ cell tumors are benign (non-cancerous), although some are cancerous and may be life threatening. The most common germ cell malignancies are maturing teratomas, dysgerminomas, and endodermal sinus tumors. Germ cell malignancies occur most often in teenagers and women in their twenties. Today, 90 percent of patients with ovarian germ cell malignancies can be cured and their fertility preserved.

Stromal Tumors—Ovarian stromal tumors are a rare class of tumors that develop from connective tissue cells that hold the ovary together and those that produce the female hormones, estrogen and progesterone. The most common types are granulosa-theca tumors and Sertoli-Leydig cell tumors. These tumors are quite rare and are usually considered low-grade cancers, with approximately 70 percent presenting as Stage I disease (cancer is limited to one or both ovaries).

Primary Peritoneal Carcinoma—The removal of one's ovaries eliminates the risk for ovarian cancer, but not the risk for a less common cancer called Primary Peritoneal Carcinoma. Primary Peritoneal Carcinoma is closely rated to epithelial ovarian cancer (most common type). It develops in cells from the peritoneum (abdominal lining) and looks the same under a microscope. It is similar in symptoms, spread and treatment.

In some examples, the breast cancer is one selected from the group comprising ductal carcinoma in situ, lobular carcinoma in situ, invasive breast cancer, invasive lobular breast cancer, Paget's disease, angiosarcoma of the breast, medulllary breast cancer, mucinous breast cancer, tubular breast cancer, adenoid cystic carcinoma of the breast, metaplastic breast cancer, lymphoma of the breast, basal type breast cancer, phyllodes or cystosarcoma phyllodes and papillary breast cancer.

In some examples, embodiment the prostate cancer is selected from the group comprising ductal adenocarcinoma, transitional cell (urothelial) cancer, squamous cell cancer, carcinoid, small cell cancer, sarcomas and sarcomatoid cancers.

### Radiolabelled Antibodies

The use of radiolabelled antibodies both for imaging as well as therapeutic radiopharmaceuticals is gaining increased interest. As RON is expressed/overexpression in many tumors, the antigen specific binding domains or the antibody molecules as disclosed in the present disclosure are also useful in generating reagents for use in diagnostic imaging.

Therefore in a tenth aspect, the present disclosure refers to a radiolabelled antibody conjugate comprising an antibody or antigen binding fragment thereof that binds RON, wherein the antibody or antigen binding fragment thereof comprises an antigen specific binding domain of the first aspect of the present disclosure. The radiolabelled antibody conjugate comprises a positron emitter, which is optionally a radio-metal such as ⁸⁹Zr (Zirconium-89). In some examples, the positron emitter may be a Fluorine-18, Gallium-68, Copper-64, Yttrium-86, Bromine-76, or Iodine-124

In some examples, the radiolabelled antibody conjugate further comprises a chelating moiety. In one example, the chelating moiety is Deferoxamine (DFO), or specifically p-SCN-Bn-Deferoxamine (DFO).

The radiolabelled antibody conjugate as disclosed herein is helpful as a positron emission tomography (PET) imaging agent. In one example of the disclosure, the radiolabelled antibody conjugate (which can be classified as an immuno-PET agent) as disclosed herein is for use as a biomarker to measure target expression (in this case RON expression) and verify optimal delivery of these agents to tumors. Antibody-drug conjugates (ADCs) combine the high affinity and specificity of mAbs with the potency of cytotoxic drugs to target tumor-expressing antigen and destroy cancer cells. In another example, the radiolabelled antibody conjugate as disclosed herein for use in immuno-PET to study the whole-body biodistribution, pharmacokinetics, and tumor targeting of anti-RON antibodies and ADCs to predict toxicity and efficacy Immuno-PET imaging using the radiolabelled antibody conjugate as disclosed herein is also useful to stratify patients who might respond or benefit from RON inhibitors or RON-targeted therapies.

In an eleventh aspect, the present disclosure also refers to a method of imaging a tissue that expresses RON, said method comprises administering a radiolabelled antibody conjugate as disclosed herein to the tissue, and visualizing RON expression by positron emission tomography (PET) imaging. In one example, the tissue is a tumour tissue, or specifically a malignant tumour tissue.

In a twelfth aspect, the present disclosure also refers to a method for treating a tumour comprising:

(a) selecting or providing a subject with a solid tumour;

(b) determining that the solid tumour is RON-positive; and

(c) administering one or more doses of a RON inhibitor to the subject;

wherein step (b) comprises: (i) administering a radiolabelled antibody conjugate as disclosed herein to the subject in need thereof; and (ii) imaging localization of the radiolabelled antibody conjugate in the tumour by positron emission tomography (PET) imaging, wherein presence of the radiolabelled antibody conjugate in the tumour indicates that the tumour is RON-positive.

In a thirteenth aspect, the present disclosure refers to a method of monitoring a cancer patient using an antibody molecule as disclosed herein, wherein the method comprises the steps of:

i. using a labelled form of the antibody molecule to access the cancer, in particular a tumour, at a first time point,

ii. using a labelled form of the antibody molecule to access the cancer, in particular a tumour, at least a second time point, and

iii. comparing the results from the two or more time points to evaluate the status of the cancer.

In some examples of the method, the labelled form of the antibody molecule is a radiolabelled antibody conjugate according to the tenth aspect of the disclosure.

In some examples of the method, wherein a decrease in RON expression by the tumour between the first time point and a subsequent time point correlates with an improved prognosis.

TABLE 1
Summary of RON antibody sequences

| | |
|---|---|
| SEQ | |
| ID | |
| NO: | Sequence |

3G4 (Kappa/light chain)

| | | |
|---|---|---|
| CDRL1 | 1 | QSIGTS |
| CDRL2 | 2 | YAS |
| CDRL3 | 3 | QQSDSWPTT |
| FR1 | 4 | DIVLTQTPAILSVSPGERVSFSCRAS |
| FR2 | 5 | IHWYQQRTNGSPRLLIK |
| FR3 | 6 | ESISGIPSRFSGSGSGTDFTLRINSVESEDLADYYC |
| FR4 | 7 | FGGGTKLEIKR |
| Light chain | 8 | DIVLTQTPAILSVSPGERVSFSCRASQSIGTSIHWYQQRTNGSPRLLI |
| variable | | KYASESISGIPSRFSGSGSGTDFTLRINSVESEDLADYYCQQSDSWP |
| (amino acid) | | TTFGGGTKLEIKR |
| Light chain | 9 | GACATTGTGCTGACCCAAACTCCAGCCATCCTGTCTGTGAGTC |
| variable | | CAGGAGAAAGAGTCAGTTTCTCCTGCAGGGCCAGTCAGAGCAT |
| (nucleotide | | TGGCACAAGCATACACTGGTATCAGCAAAGAACAAATGGTTCT |
| sequence) | | CCAAGGCTTCTCATAAAATATGCTTCTGAGTCTATCTCTGGGAT |
| | | CCCTTCCAGGTTTAGTGGCAGTGGATCAGGGACAGATTTTACT |
| | | CTTCGCATCAACAGTGTGGAGTCTGAGGATCTTGCAGATTATT |
| | | ACTGTCAGCAAAGTGATAGCTGGCCAACCACGTTCGGAGGGG |
| | | GGACCAAGCTGGAAATAAAACG |

3G4 (Heavy chain)

| | | |
|---|---|---|
| CDRH1 | 10 | GYTFTSYY |
| CDRH2 | 11 | IYPGNVNT |
| CDRH3 | 12 | ARLGYYRYGGAMDY |
| FR1 | 13 | EVQLQESGPELVKPGTSVRISCKAS |
| FR2 | 14 | MHWVKQRPGQGLEWIGY |
| FR3 | 15 | KYNEKFKGKATLTADKSSSTAYMQLSSLTSEDSAVYFC |
| FR4 | 16 | WGQGTAVTVSS |
| Heavy chain | 17 | EVQLQESGPELVKPGTSVRISCKASGYTFTSYYMHWVKQRPGQG |
| variable | | LEWIGYIYPGNVNTKYNEKFKGKATLTADKSSSTAYMQLSSLTSE |
| (amino acid) | | DSAVYFCARLGYYRYGGAMDYWGQGTAVTVSS |
| Heavy chain | 18 | GAGGTACAGCTGCAGGAGTCAGGACCTGAGCTGGTGAAGCCT |
| variable | | GGGACTTCAGTGAGGATATCCTGCAAGGCTTCTGGCTACACCT |
| (nucleotide | | TCACAAGCTACTATATGCATTGGGTGAAGCAGAGGCCTGGACA |
| sequence) | | GGGACTTGAGTGGATTGGATACATTTATCCTGGAAATGTTAAT |
| | | ACTAAATACAATGAGAAGTTCAAGGGCAAGGCCACACTGACT |
| | | GCAGACAAATCCTCCAGCACAGCCTACATGCAACTCAGCAGCC |
| | | TGACCTCTGAGGACTCTGCGGTCTATTTCTGTGCAAGGTTGGGC |
| | | TACTATAGGTACGGGGGTGCTATGGACTACTGGGGTCAAGGAA |
| | | CCGCAGTCACCGTCTCCTCA |

3F6 (Kappa/light chain)

| | | |
|---|---|---|
| CDRL1 | 19 | QDISNY |
| CDRL2 | 20 | YTS |
| CDRL3 | 21 | QQYNKLPWT |
| FR1 | 22 | DIVLTQTPSSLSASLGDRVTISCSAS |
| FR2 | 23 | LNWYQQKPDGIVKLLIY |
| FR3 | 24 | SLHSGVPSRFSGSGSGTDFSLTISNLEPEDIATYYC |
| FR4 | 25 | FGGGTKLEIK |
| Light chain | 26 | DIVLTQTPSSLSASLGDRVTISCSASQDISNYLNWYQQKPDGIVKL |
| variable | | LIYYTSSLHSGVPSRFSGSGSGTDFSLTISNLEPEDIATYYCQQYNK |
| (amino acid) | | LPWTFGGGTKLEIK |
| Light chain | 27 | GATATTGTGCTGACCCAGACTCCATCCTCCCTGTCTGCCTCTCT |
| variable | | GGGAGACAGAGTCACCATCAGTTGCAGTGCAAGTCAGGACATT |
| (nucleotide | | AGCAATTATTTAAACTGGTATCAGCAGAAACCAGATGGAATTG |
| sequence) | | TTAAACTCCTGATCTATTACACATCAAGTTTACACTCAGGAGTC |
| | | CCATCAAGGTTCAGTGGCAGTGGGTCTGGGACAGATTTTTCTC |
| | | TCACCATCAGCAACCTGGAACCTGAAGATATTGCCACTTACTA |
| | | TTGTCAGCAGTATAATAAACTTCCGTGGACATTCGGTGGAGGC |
| | | ACCAAGCTGGAAATCAAAC |

3F6 (Heavy chain)

| | | |
|---|---|---|
| CDRH1 | 28 | GYSITSGYY |
| CDRH2 | 29 | MSYDGSN |
| CDRH3 | 30 | SRALAYYGNFDY |
| FR1 | 31 | LEVKLEESGPGLVKPSQSLSLTCSVT |
| FR2 | 32 | WNWIRQFPGNKLEWMGF |
| FR3 | 33 | NYNPSLKNRISITRDTSKNQFFLKLNSVTSEDTATYYC |
| FR4 | 34 | WGQGTTLTVSS |
| Heavy chain | 35 | LEVKLEESGPGLVKPSQSLSLTCSVTGYSITSGYYWNWIRQFPGN |
| variable | | KLEWMGFMSYDGSNNYNPSLKNRISITRDTSKNQFFLKLNSVTSE |
| (amino acid) | | DTATYYCSRALAYYGNFDYWGQGTTLTVSS |
| Heavy chain | 36 | CTTGAGGTAAAGCTGGAGGAGTCAGGACCTGGCCTCGTGAAAC |
| variable | | CTTCTCAGTCTCTGTCTCTCACCTGCTCTGTCACTGGCTACTCC |
| (nucleotide | | ATCACCAGTGGTTATTACTGGAACTGGATCCGGCAATTTCCAG |
| sequence) | | GAAACAAACTGGAATGGATGGGTTTCATGAGCTACGACGGTA |
| | | GCAATAACTACAACCCATCTCTCAAAAATCGAATCTCCATCAC |
| | | TCGTGACACATCTAAGAACCAGTTTTTCCTGAAATTGAATTCTG |
| | | TGACTTCTGAGGACACAGCTACATATTACTGTTCAAGAGCTCT |
| | | AGCCTACTATGGTAACTTTGACTACTGGGGCCAAGGCACCACT |
| | | CTCACAGTCTCCTCA |

3F8 (Kappa/light chain)

| | | |
|---|---|---|
| CDRL1 | 37 | QNVNTN |
| CDRL2 | 38 | SAS |
| CDRL3 | 39 | QQYNTYPLT |
| FR1 | 40 | DIVLTQTPKVMSTSVGDRVSVTCKAS |
| FR2 | 41 | VAWYQQKPGQSPKALIY |
| FR3 | 42 | SRYSGVPDRFTGSGSGTDFTLTISNVQSEDLADYFC |
| FR4 | 43 | FAAGTKLELK |
| Light chain | 44 | DIVLTQTPKVMSTSVGDRVSVTCKASQNVNTNVAWYQQKPGQSP |
| variable | | KALIYSASSRYSGVPDRFTGSGSGTDFTLTISNVQSEDLADYFCQQ |
| (amino acid) | | YNTYPLTFAAGTKLELK |
| Light chain | 45 | GACATTGTGCTCACACAAACTCCAAAAGTCATGTCCACATCAG |
| variable | | TAGGAGACAGGGTCAGCGTCACCTGCAAGGCCAGTCAGAATG |
| (nucleotide | | TTAATACTAATGTAGCCTGGTATCAACAGAAACCAGGGCAATC |
| sequence) | | TCCTAAAGCACTCATTTACTCGGCATCCTCCCGGTACAGTGGA |
| | | GTCCCTGATCGCTTCACAGGCAGTGGATCTGGGACAGATTTCA |
| | | CTCTCACCATCAGCAATGTGCAGTCTGAAGACTTGGCTGACTA |
| | | TTTCTGTCAGCAATATAACACCTATCCGCTCACGTTCGCTGCTG |
| | | GGACCAAGCTGGAGCTAAAAC |

3F8 (Heavy chain)

| | | |
|---|---|---|
| CDRH1 | 46 | GFTFSSFW |
| CDRH2 | 47 | IRLKSNNYAT |
| CDRH3 | 48 | IPPYYYTVDY |
| FR1 | 49 | EVKLQESGGGLVQPGRSLKLSCVAS |
| FR2 | 50 | MHWVRQSPEKGLEWVAE |
| FR3 | 51 | HYAESVKGRFTISRDDSKSSVHLQMNNLRTEDTGIYYC |
| FR4 | 52 | WGQGTSVTVSS |
| Heavy chain | 53 | EVKLQESGGGLVQPGRSLKLSCVASGFTFSSFWMHWVRQSPEKG |
| variable | | LEWVAEIRLKSNNYATHYAESVKGRFTISRDDSKSSVHLQMNNL |
| (amino acid) | | RTEDTGIYYCIPPYYYTVDYWGQGTSVTVSS |
| Heavy chain | 54 | GAAGTGAAGCTGCAGGAGTCTGGAGGAGGCTTGGTGCAACCT |
| variable | | GGAAGATCCTTGAAACTCTCCTGTGTTGCCTCTGGATTCACTTT |
| (nucleotide | | CAGTAGTTTCTGGATGCACTGGGTCCGCCAGTCTCCAGAGAAG |
| sequence) | | GGTCTTGAGTGGGTTGCTGAAATTCGATTGAAATCTAATAATT |
| | | ATGCAACACATTATGCGGAGTCTGTGAAAGGGAGGTTCACCAT |
| | | CTCAAGAGATGATTCCAAAAGTAGCGTCCACCTGCAAATGAAC |
| | | AACTTAAGAACTGAAGACACTGGCATTTATTACTGTATCCCAC |
| | | CCTATTACTATACTGTGGACTACTGGGGTCAAGGAACCTCAGT |
| | | CACCGTCTCCTCA |

4A7 (Kappa/light chain)

| | | |
|---|---|---|
| CDRL1 | 1 | QSIGTS |
| CDRL2 | 2 | YAS |
| CDRL3 | 3 | QQSDSWPTT |
| FR1 | 4 | DIVLTQTPAILSVSPGERVSFSCRAS |
| FR2 | 5 | IHWYQQRTNGSPRLLIK |
| FR3 | 6 | ESISGIPSRFSGSGSGTDFTLRINSVESEDLADYYC |
| FR4 | 7 | FGGGTKLEIKR |
| Light chain | 8 | DIVLTQTPAILSVSPGERVSFSCRASQSIGTSIHWYQQRTNGSPRLLI |
| variable | | KYASESISGIPSRFSGSGSGTDFTLRINSVESEDLADYYCQQSDSWP |
| (amino acid) | | TTFGGGTKLEIKR |
| Light chain | 55 | GACATTGTGATGACACAATCTACAGCCATCCTGTCTGTGAGTC |
| variable | | CAGGAGAAAGAGTCAGTTTCTCCTGCAGGGCCAGTCGGAGCAT |
| (nucleotide | | TGGCACAAGCATACACTGGTATCAGCAAAGAACAAATGGTTCT |
| sequence) | | CCAAGGCTTCTCATAAAGTATGCTTCTGAGTCTATCTCTGGGAT |
| | | CCCTTCCAGGTTTAGTGGCAGTGGATCAGGGACAGATTTTACT |
| | | CTTCGCATCAACAGTGTGGAGTCTGAAGATCTTGCAGATTATT |
| | | ACTGTCAACAAAGTGATAGCTGGCCAACCACGTTCGGAGGGG |
| | | GGACCAAGCTGGAAATAAAACG |

4A7 (Heavy chain)

| | | |
|---|---|---|
| CDRH1 | 10 | GYTFTSYY |
| CDRH2 | 11 | IYPGNVNT |
| CDRH3 | 12 | ARLGYYRYGGAMDY |
| FR1 | 56 | QVKLQESGPELVKPGTSVRISCKAS |
| FR2 | 57 | IHWVKQRPGQGLEWIGW |
| FR3 | 15 | KYNEKFKGKATLTADKSSSTAYMQLSSLTSEDSAVYFC |
| FR4 | 52 | WGQGTSVTVSS |
| Heavy chain | 58 | QVKLQESGPELVKPGTSVRISCKASGYTFTSYYIHWVKQRPGQGL |
| variable | | EWIGWIYPGNVNTKYNEKFKGKATLTADKSSSTAYMQLSSLTSE |
| (amino acid) | | DSAVYFCARLGYYRYGGAMDYWGQGTSVTVSS |
| Heavy chain | 59 | CAGGTGAAGCTGCAGGAGTCAGGACCTGAGCTGGTGAAGCCT |
| variable | | GGGACTTCAGTGAGGATATCCTGCAAGGCTTCTGGCTACACCT |
| (nucleotide | | TCACAAGCTACTATATACATTGGGTGAAGCAGAGGCCTGGACA |
| sequence) | | GGGACTTGAGTGGATTGGATGGATTTATCCTGGAAATGTTAAT |
| | | ACTAAGTACAATGAGAAGTTCAAGGGCAAGGCCACACTGACT |
| | | GCAGACAAATCCTCCAGCACAGCCTACATGCAACTCAGCAGCC |
| | | TGACCTCTGAGGACTCTGCGGTCTATTTCTGTGCAAGGTTGGGC |
| | | TACTATAGGTACGGGGGTGCTATGGACTACTGGGGTCAAGGAA |
| | | CCTCAGTCACCGTCTCCTCA |

6A9 (Kappa/light chain)

| | | |
|---|---|---|
| CDRL1 | 60 | QDISKY |
| CDRL2 | 20 | YTS |
| CDRL3 | 61 | QQGNTLPRT |
| FR1 | 62 | DIVLTQSPSSLSASLGDRVTITCRAS |
| FR2 | 63 | LNWYQQKPDGTVKLLIY |
| FR3 | 64 | TLHSGVPSRFSGSGSGTDFSLTITNLEQEDIATYFC |
| FR4 | 25 | FGGGTKLEIK |
| Light chain | 65 | DIVLTQSPSSLSASLGDRVTITCRASQDISKYLNWYQQKPDGTVKL |
| variable | | LIYYTSTLHSGVPSRFSGSGSGTDFSLTITNLEQEDIATYFCQQGNT |
| (amino acid) | | LPRTFGGGTKLEIK |
| Light chain | 66 | GATATTGTGCTCACCCAATCTCCATCCTCCCTGTCTGCCTCTCT |
| variable | | GGGAGACAGAGTCACCATCACTTGCAGGGCAAGTCAGGACAT |
| (nucleotide | | TAGCAAATACTTAAACTGGTATCAACAGAAACCAGATGGAACT |
| sequence) | | GTTAAACTCCTGATCTACTACACATCAACATTACATTCAGGAG |
| | | TCCCATCAAGGTTCAGTGGCAGTGGGTCTGGGACAGATTTTTC |
| | | TCTCACCATTACCAACCTGGAGCAAGAAGATATTGCCACTTAC |
| | | TTTTGCCAACAGGGCAATACTCTTCCTCGGACGTTCGGTGGAG |
| | | GCACCAAGCTGGAAATCAAAC |

6A9 (Heavy chain)

| | | |
|---|---|---|
| CDRH1 | 67 | GSSITSDYA |
| CDRH2 | 68 | IGYSVGT |
| CDRH3 | 69 | ARRPLYFYYAMDF |
| FR1 | 70 | LEVKLEESGPGLLKPSQSLSLTCTVT |
| FR2 | 71 | WNWIRQFPGNKLEWMGY |
| FR3 | 72 | SHNPSLKGRISITRDTSKNQFFLHLNSVTTEDTAIYYC |
| FR4 | 52 | WGQGTSVTVSS |
| Heavy chain | 73 | LEVKLEESGPGLLKPSQSLSLTCTVTGSSITSDYAWNWIRQFPGNK |
| variable | | LEWMGYIGYSVGTSHNPSLKGRISITRDTSKNQFFLHLNSVTTEDT |
| (amino acid) | | AIYYCARRPLYFYYAMDFWGQGTSVTVSS |
| Heavy chain | 74 | CTTGAGGTGAAGCTGGAGGAGTCTGGACCTGGCCTGTTGAAAC |
| variable | | CTTCTCAGTCTCTGTCCCTCACCTGCACTGTCACTGGCTCCTCA |
| (nucleotide | | ATCACCAGTGATTATGCCTGGAACTGGATCCGGCAGTTTCCAG |
| sequence) | | GAAACAAACTGGAGTGGATGGGCTACATAGGTTACAGTGTTGG |
| | | CACTAGCCACAACCCATCTCTCAAAGGTCGAATCTCTATCACT |
| | | CGAGACACATCCAAGAACCAGTTCTTCCTGCACTTGAATTCTG |
| | | TGACTACTGAGGACACAGCCATATATTACTGTGCAAGACGCCC |
| | | TCTCTACTTCTACTATGCTATGGACTTCTGGGGTCAAGGAACCT |
| | | CAGTCACCGTCTCCTCA |

7H11 (Kappa/light chain)

| | | |
|---|---|---|
| CDRL1 | 75 | KSVSTSAYNF |
| CDRL2 | 76 | LAS |
| CDRL3 | 77 | QHSRELPYT |
| FR1 | 78 | DIVLTQSPVSLAVSLGQRATISCRAS |
| FR2 | 79 | MHWYQQKPGQPPKLLIY |
| FR3 | 80 | NLESGVPARLSGSGSGTDFTLNIHPVEEEDAATYYC |
| FR4 | 7 | FGGGTKLEIKR |
| Light chain | 81 | DIVLTQSPVSLAVSLGQRATISCRASKSVSTSAYNFMHWYQQKPG |
| variable | | QPPKLLIYLASNLESGVPARLSGSGSGTDFTLNIHPVEEEDAATY |
| (amino acid) | | CYQHSRELPYTFGGGTKLEIKR |
| Light chain | 82 | GACATTGTGCTGACACAGTCTCCTGTTTCTTTAGCTGTATCTC |
| variable | | TGGGGCAGAGGGCCACCATCTCATGCAGGGCCAGCAAAAGTGT |
| (nucleotide | | CAGTACCTCTGCCTATAATTTTATGCACTGGTACCAGCAGAAA |
| sequence) | | CCAGGACAGCCACCCAAACTCCTCATCTATCTTGCATCCAACC |
| | | TAGAATCTGGGGTCCCTGCCAGGCTCAGTGGCAGTGGGTCTGG |
| | | GACAGACTTCACCCTCAACATCCATCCTGTGGAGGAGGAGGAT |
| | | GCTGCAACCTATTACTGTCAGCACAGTAGGGAGCTTCCGTACA |
| | | CGTTCGGAGGGGGGACCAAGCTGGAAATAAAACG |

7H11 (Heavy chain)

| | | |
|---|---|---|
| CDRH1 | 83 | GYTFTDYN |
| CDRH2 | 84 | IYPYNGNS |
| CDRH3 | 85 | ASLYYLGYFDV |
| FR1 | 86 | EVKLEQSGPELVKPGASVKISCKAS |
| FR2 | 87 | MHWVRQPPGQSLEWIGF |
| FR3 | 88 | GYNPNFKTKATLTMDISSSTAYMEVRNLTSEDSATYFC |
| FR4 | 89 | WGAGTTVTVSS |
| Heavy chain | 90 | EVKLEQSGPELVKPGASVKISCKASGYTFTDYNMHWVRQPPGQS |
| variable | | LEWIGFIYPYNGNSGYNPNFKTKATLTMDISSSTAYMEVRNLTSE |
| (amino acid) | | DSATYFCASLYYLGYFDVWGAGTTVTVSS |
| Heavy chain | 91 | GAAGTGAAGCTGGAGCAGTCTGGACCTGAGCTGGTGAAACCT |
| variable | | GGGGCCTCAGTGAAGATATCCTGCAAGGCTTCTGGATATACAT |
| (nucleotide | | TCACTGACTACAACATGCACTGGGTGAGGCAGCCCCCTGGACA |
| sequence) | | GAGCCTTGAGTGGATTGGATTTATTTATCCTTACAATGGTAATT |
| | | CTGGCTACAATCCGAACTTCAAGACCAAGGCCACATTGACCAT |
| | | GGACATTTCCTCCAGCACAGCCTACATGGAGGTCCGCAACCTG |
| | | ACATCTGAGGATTCTGCGACCTATTTCTGTGCAAGTCTTTATTA |
| | | CCTCGGATACTTCGATGTGTGGGGCGCAGGGACCACGGTCACC |
| | | GTCTCCTCA |

8B7 (Kappa/light chain)

| | | |
|---|---|---|
| CDRL1 | 1 | QSIGTS |
| CDRL2 | 2 | YAS |
| CDRL3 | 3 | QQSDSWPTT |
| FR1 | 92 | DIVLTQSTAILSVSPGERVSFSCRAS |
| FR2 | 5 | IHWYQQRTNGSPRLLIK |
| FR3 | 6 | ESISGIPSRFSGSGSGTDFTLRINSVESEDLADYYC |
| FR4 | 7 | FGGGTKLEIKR |
| Light chain | 93 | DIVLTQSTAILSVSPGERVSFSCRASQSIGTSIHWYQQRTNGSPRLLI |
| variable | | KYASESISGIPSRFSGSGSGTDFTLRINSVESEDLADYYCQQSDSWP |
| (amino acid) | | TTFGGGTKLEIKR |
| Light chain | 94 | GACATTGTGCTGACCCAGTCTACAGCCATCCTGTCTGTGAGTCC |
| variable | | AGGAGAAAGAGTCAGTTTCTCCTGCAGGGCCAGTCAGAGCATT |
| (nucleotide | | GGCACAAGCATACACTGGTATCAGCAAAGAACAAATGGTTCTC |
| sequence) | | CAAGGCTTCTCATAAAATATGCTTCTGAGTCTATCTCTGGGATC |
| | | CCTTCCAGGTTTAGTGGCAGTGGATCAGGGACAGATTTTACTC |
| | | TTCGCATCAACAGTGTGGAGTCTGAGGATCTTGCAGATTATTA |
| | | CTGTCAGCAAAGTGATAGCTGGCCAACCACGTTCGGAGGGGG |
| | | GACCAAGCTGGAAATAAAACG |

8B7 (Heavy chain)

| | | |
|---|---|---|
| CDRH1 | 10 | GYTFTSYY |
| CDRH2 | 11 | IYPGNVNT |
| CDRH3 | 12 | ARLGYYRYGGAMDY |
| FR1 | 95 | EVQLEESGPELVKPGTSVRISCKAS |
| FR2 | 14 | MHWVKQRPGQGLEWIGY |
| FR3 | 15 | KYNEKFKGKATLTADKSSSTAYMQLSSLTSEDSAVYFC |
| FR4 | 52 | WGQGTSVTVSS |
| Heavy chain | 96 | EVQLEESGPELVKPGTSVRISCKASGYTFTSYYMHWVKQRPGQG |
| variable | | LEWIGYIYPGNVNTKYNEKFKGKATLTADKSSSTAYMQLSSLTSE |
| (amino acid) | | DSAVYFCARLGYYRYGGAMDYWGQGTSVTVSS |
| Heavy chain | | |
| variable | | GGACTTCAGTGAGGATATCCTGCAAGGCTTCTGGCTACACCTT |
| (nucleotide | | CACAAGCTACTATATGCATTGGGTGAAGCAGAGGCCTGGACAG |
| sequence) | | GGACTTGAGTGGATTGGATACATTTATCCTGGAAATGTTAATA |
| | | CTAAATACAATGAGAAGTTCAAGGGCAAGGCCACACTGACTG |
| | | CAGACAAATCCTCCAGCACAGCCTACATGCAACTCAGCAGCCT |
| | | GACCTCTGAGGACTCTGCGGTCTATTTCTGTGCAAGGTTGGGCT |
| | | ACTATAGGTACGGGGGTGCTATGGACTACTGGGGTCAAGGAAC |
| | | CTCAGTCACCGTCTCCTCA |

9F2 (Kappa/light chain)

| | | |
|---|---|---|
| CDRL1 | 98 | SSVSY |
| CDRL2 | 99 | DTS |
| CDRL3 | 100 | FQGNKYPLT |
| FR1 | 101 | DIVLTQTTAIMSASPGEKVTMTCSAS |
| FR2 | 102 | IHWYQQKSSTSPKLWIY |
| FR3 | 103 | KLASGVPGRFSGSGSGNSYSLTISSMEAEDVATYYC |
| FR4 | 104 | FGAGTKLELK |
| Light chain | 105 | DIVLTQTTAIMSASPGEKVTMTCSASSSVSYIHWYQQKSSTSPKL |
| variable | | WIYDTSKLASGVPGRFSGSGSGNSYSLTISSMEAEDVATYYCFQG |
| (amino acid) | | NKYPLTFGAGTKLELK |
| Light chain | 106 | GACATTGTGCTGACACAAACTACAGCAATCATGTCTGCATCTC |
| variable | | CAGGGGAAAAGGTCACCATGACCTGCAGTGCCAGCTCAAGTGT |
| (nucleotide | | AAGTTACATACACTGGTACCAGCAGAAGTCAAGCACCTCCCCC |
| sequence) | | AAACTCTGGATTTATGACACATCCAAACTGGCTTCTGGAGTCC |
| | | CAGGTCGCTTCAGTGGCAGTGGGTCTGGAAACTCTTACTCTCTC |
| | | ACGATCAGCAGCATGGAGGCTGAAGATGTTGCCACTTACTACT |
| | | GTTTTCAGGGGAATAAGTACCCGCTCACGTTCGGTGCTGGGAC |
| | | CAAGTTGGAGCTGAAAC |

9F2 (Heavy chain)

| | | |
|---|---|---|
| CDRH1 | 107 | GFSLTSYG |
| CDRH2 | 108 | IWAGGTT |
| CDRH3 | 109 | ASYDYDGYFDV |
| FR1 | 110 | QVKLQQSGPGLVAPSQSLSITCTVS |
| FR2 | ill | VHWVRQPPGKGLEWLGV |
| FR3 | 112 | NYNSALMSRLSISKDNSKSQVFLKMNSLQTDDTAMYYC |
| FR4 | 89 | WGAGTTVTVSS |
| Heavy chain | 113 | QVKLQQSGPGLVAPSQSLSITCTVSGFSLTSYGVHWVRQPPGKGL |
| variable | | EWLGVIWAGGTTNYNSALMSRLSISKDNSKSQVFLKMNSLQTDD |
| (amino acid) | | TAMYYCASYDYDGYFDVWGAGTTVTVSS |
| Heavy chain | 114 | CAGGTCAAGCTGCAGCAGTCAGGACCTGGCCTGGTGGCGCCCT |
| variable | | CACAGAGCCTGTCCATCACTTGCACTGTCTCTGGCTTTTCATTA |
| (nucleotide | | ACCAGCTATGGTGTACACTGGGTTCGCCAGCCTCCAGGAAAGG |
| sequence) | | GTCTGGAGTGGCTGGGAGTCATATGGGCTGGTGGAACCACAAA |
| | | TTATAATTCGGCTCTCATGTCTAGACTGAGCATCAGCAAAGAC |
| | | AATTCCAAGAGCCAAGTTTTCTTAAAAATGAACAGTCTGCAAA |
| | | CTGATGACACAGCCATGTACTACTGTGCCAGTTATGATTACGA |
| | | CGGGTACTTCGATGTCTGGGGCGCAGGGACCACGGTCACCGTC |
| | | TCCTCA |

9F6 (Kappa/light chain)

| | | |
|---|---|---|
| CDRL1 | 115 | QDIGSN |
| CDRL2 | 116 | ATS |
| CDRL3 | 117 | LQYISSPFT |
| FR1 | 118 | DIVLTQTPSSLSASLGERVSLTCRAS |
| FR2 | 119 | LNWLQQEPDGTIKRLIY |
| FR3 | 120 | SLDPGVPKRFSGSRSGSDYSLTISSLESEDFVDYYC |
| FR4 | 121 | FGSGTKLEIK |
| Light chain | 122 | DIVLTQTPSSLSASLGERVSLTCRASQDIGSNLNWLQQEPDGTIKR |
| variable | | LIYATSSLDPGVPKRFSGSRSGSDYSLTISSLESEDFVDYYCLQYI |
| (amino acid) | | SSPFTFGSGTKLEIK |
| Light chain | 123 | GACATTGTGCTCACCCAGACTCCATCCTCCTTATCTGCCTCTCT |
| variable | | GGGAGAAAGAGTCAGTCTCACTTGTCGGGCAAGTCAGGACATT |
| (nucleotide | | GGTAGTAACTTAAACTGGCTTCAGCAGGAACCAGATGGAACTA |
| sequence) | | TTAAACGCCTGATCTACGCCACATCCAGTTTAGATCCAGGTGT |
| | | CCCCAAAAGGTTCAGTGGCAGTAGGTCTGGGTCAGATTATTCT |
| | | CTCACCATCAGCAGCCTTGAGTCTGAAGATTTTGTAGACTATTA |
| | | CTGTCTACAATATATTAGTTCTCCATTCACGTTCGGCTCGGGGA |
| | | CAAAGTTGGAAATAAAAC |

9F6 (Heavy chain)

| | | |
|---|---|---|
| CDRH1 | 124 | GYSFTGYY |
| CDRH2 | 125 | ISCYNGAT |
| CDRH3 | 126 | ARGGTTATPFDY |
| FR1 | 127 | QVKLEESGPELVKTGASVKISCKAS |
| FR2 | 128 | IHWVKQSHGQSLEWIGY |
| FR3 | 129 | RYNQKFKGKAAFTVDTSSSTAYLQFNSLTSEDSAIYYC |
| FR4 | 34 | WGQGTTLTVSS |
| Heavy chain | 130 | QVKLEESGPELVKTGASVKISCKASGYSFTGYYIHWVKQSHGQSL |
| variable | | EWIGYISCYNGATRYNQKFKGKAAFTVDTSSSTAYLQFNSLTSED |
| (amino acid) | | SAIYYCARGGTTATPFDYWGQGTTLTVSS |
| Heavy chain | 131 | CAGGTTAAGCTGGAGGAGTCAGGACCTGAGCTAGTGAAGACT |
| variable | | GGGGCTTCAGTGAAGATATCCTGCAAGGCTTCTGGTTACTCAT |
| (nucleotide | | TCACTGGTTACTACATACACTGGGTCAAGCAGAGCCATGGACA |
| sequence) | | GAGCCTTGAGTGGATTGGATATATTAGCTGTTACAACGGTGCT |
| | | ACTAGATATAACCAGAAGTTCAAGGGCAAGGCCGCATTTACTG |
| | | TAGACACATCCTCCAGCACAGCCTACTTACAGTTCAACAGCCT |
| | | GACATCTGAAGACTCTGCGATCTATTACTGTGCAAGAGGGGGT |
| | | ACTACGGCTACGCCCTTTGACTACTGGGGCCAAGGCACCACTC |
| | | TCACAGTCTCCTCA |

10G1 (Kappa/light chain)

| | | |
|---|---|---|
| CDRL1 | 132 | QDINNY |
| CDRL2 | 133 | HAD |
| CDRL3 | 134 | LQYDDFPPFT |
| FR1 | 135 | DIVLTQTPSSMYAFLGERVTIICKAS |
| FR2 | 136 | LNWVQQKPGKAPKTLIY |
| FR3 | 137 | RLADGVPSRFSGSGSGQDYSLTISSLEYEDMGIYYC |
| FR4 | 121 | FGSGTKLEIK |
| Light chain | 138 | DIVLTQTPSSMYAFLGERVTIICKASQDINNYLNWVQQKPGKAPK |
| variable | | TLIYHADRLADGVPSRFSGSGSGQDYSLTISSLEYEDMGIYYCLQ |
| (amino acid) | | YDDFPPFTFGSGTKLEIK |
| Light chain | 139 | GACATTGTGCTGACCCAAACTCCATCTTCCATGTATGCATTTCT |
| variable | | AGGAGAGAGAGTCACCATCATTTGCAAGGCGAGTCAGGACAT |
| (nucleotide | | CAATAACTATTTAAACTGGGTCCAGCAGAAACCAGGGAAAGCT |
| sequence) | | CCTAAGACCCTGATCTATCATGCAGACAGATTGGCAGATGGGG |
| | | TCCCATCAAGGTTCAGTGGCAGTGGGTCTGGGCAAGATTATTC |
| | | TCTCACCATCAGCAGCCTGGAATATGAAGATATGGGAATTTAT |
| | | TATTGTCTACAGTATGATGACTTTCCTCCATTCACGTTCGGCTC |
| | | GGGGACAAAGTTGGAAATAAAAC |

10G1 (Heavy chain)

| | | |
|---|---|---|
| CDRH1 | 140 | GFTFSSFG |
| CDRH2 | 141 | ISGGSGTF |
| CDRH3 | 142 | ARSTTVLDY |
| FR1 | 143 | EVKLEQSGGGLVQPGGSRKLSCAAS |
| FR2 | 144 | MHWVRQAPEKGLEWVAY |
| FR3 | 145 | YYADTVKGRFTISRDNPKNTLFLQMTSLRSEDTAIYYC |
| FR4 | 34 | WGQGTTLTVSS |
| Heavy chain | 146 | EVKLEQSGGGLVQPGGSRKLSCAASGFTFSSFGMHWVRQAPEKG |
| variable | | LEWVAYISGGSGTFYYADTVKGRFTISRDNPKNTLFLQMTSLRS |
| (amino acid) | | DETAIYYCARSTTVLDYWGQGTTLTVSS |
| Heavy chain | 147 | GAAGTTAAGCTGGAGCAGTCTGGGGGAGGCTTAGTGCAGCCTG |
| variable | | GAGGGTCCCGGAAACTCTCCTGTGCAGCCTCTGGATTCACTTTC |
| (nucleotide | | AGTAGCTTTGGGATGCACTGGGTTCGTCAGGCTCCAGAGAAGG |
| sequence) | | GGCTGGAGTGGGTCGCATATATTAGTGGTGGCAGTGGTACCTT |
| | | CTACTATGCAGACACAGTGAAGGGCCGATTCACCATCTCCAGA |
| | | GACAATCCCAAGAACACCCTATTCCTGCAAATGACCAGTCTAA |
| | | GGTCTGAGGACACGGCCATTTATTACTGTGCAAGATCGACTAC |
| | | GGTTCTTGACTATTGGGGCCAAGGCACCACTCTCACAGTCTCC |
| | | TCA |

SEQ ID NO: 148-
Full length RON protein amino acid sequence
MELLPPLPQSFLLLLLLPAKPAAGEDWQCPRTPYA
ASRDFDVKYVVPSFSAGGLVQAMVTYEGDRNESAV
FVAIRNRLHVLGPDLKSVQSLATGPAGDPGCQTCA
ACGPGPHGPPGDTDTKVLVLDPALPALVSCGSSLQ
GRCFLHDLEPQGTAVHLAAPACLFSAHHNRPDDCP
DCVASPLGTRVTVVEQGQASYFYVASSLDAAVAAS
FSPRSVSIRRLKADASGFAPGFVALSVLPKHLVSY
SIEYVHSFHTGAFVYFLTVQPASVTDDPSALHTRL
ARLSATEPELGDYRELVLDCRFAPKRRRRGAPEGG
QPYPVLRVAHSAPVGAQLATELSIAEGQEVLFGVF
VTGKDGGPGVGPNSVVCAFPIDLLDTLIDEGVERC
CESPVHPGLRRGLDFFQSPSFCPNPPGLEALSPNT
SCRHFPLLVSSSFSRVDLFNGLLGPVQVTALYVTR
LDNVTVAHMGTMDGRILQVELVRSLNYLLYVSNFS
LGDSGQPVQRDVSRLGDHLLFASGDQVFQVPIQGP
GCRHFLTCGRCLRAWHFMGCGWCGNMCGQQKECPG
SWQQDHCPPKLTEFHPHSGPLRGSTRLTLCGSNFY
LHPSGLVPEGTHQVTVGQSPCRPLPKDSSKLRPVP
RKDFVEEFECELEPLGTQAVGPTNVSLTVTNMPPG
KHFRVDGTSVLRGFSFMEPVLIAVQPLFGPRAGGT
CLTLEGQSLSVGTSRAVLVNGTECLLARVSEGQLL
CATPPGATVASVPLSLQVGGAQVPGSWTFQYREDP
VVLSISPNCGYINSHITICGQHLTSAWHLVLSFHD
GLRAVESRCERQLPEQQLCRLPEYVVRDPQGWVAG
NLSARGDGAAGFTLPGFRFLPPPHPPSANLVPLKP
EEHAIKFEYIGLGAVADCVGINVTVGGESCQHEFR
GDMVVCPLPPSLQLGQDGAPLQVCVDGECHILGRV
VRPGPDGVPQSTLLGILLPLLLLVAALATALVFSY
WWRRKQLVLPPNLNDLASLDQTAGATPLPILYSGS
DYRSGLALPAIDGLDSTTCVHGASFSDSEDESCVP
LLRKESIQLRDLDSALLAEVKDVLIPHERVVTHSD
RVIGKGHFGVVYHGEYIDQAQNRIQCAIKSLSRIT
EMQQVEAFLREGLLMRGLNHPNVLALIGIMLPPEG
LPHVLLPYMCHGDLLQFIRSPQRNPTVKDLISFGL
QVARGMEYLAEQKFVHRDLAARNCMLDESFTVKVA
DFGLARDILDREYYSVQQHRHARLPVKWMALESLQ
TYRFTTKSDVWSFGVLLWELLTRGAPPYRHIDPFD
LTHFLAQGRRLPQPEYCPDSLYQVMQQCWEADPAV
RPTFRVLVGEVEQIVSALLGDHYVQLPATYMNLGP
STSHEMNVRPEQPQFSPMPGNVRRPRPLSEPPRPT
SEQ ID NO: 149- Full length RON
extracellular domain (ECD) amino acid
sequence
(residues 25-957 of full-length RON
protein amino acid sequence, SEQ ID NO: 148)
EDWQCPRTPYAASRDFDVKYVVPSFSAGGLVQAMV
TYEGDRNESAVFVAIRNRLHVLGPDLKSVQSLATG
PAGDPGCQTCAACGPGPHGPPGDTDTKVLVLDPAL
PALVSCGSSLQGRCFLHDLEPQGTAVHLAAPACLF
SAHHNRPDDCPDCVASPLGTRVTVVEQGQASYFYV
ASSLDAAVAASFSPRSVSIRRLKADASGFAPGFVA
LSVLPKHLVSYSIEYVHSFHTGAFVYFLTVQPASV
TDDPSALHTRLARLSATEPELGDYRELVLDCRFAP
KRRRRGAPEGGQPYPVLRVAHSAPVGAQLATELSI
AEGQEVLFGVFVTGKDGGPGVGPNSVVCAFPIDLL
DTLIDEGVERCCESPVHPGLRRGLDFFQSPSFCPN
PPGLEALSPNTSCRHFPLLVSSSFSRVDLFNGLLG
PVQVTALYVTRLDNVTVAHMGTMDGRILQVELVRS
LNYLLYVSNFSLGDSGQPVQRDVSRLGDHLLFASG
DQVFQVPIQGPGCRHFLTCGRCLRAWHFMGCGWCG
NMCGQQKECPGSWQQDHCPPKLTEFHPHSGPLRGS
TRLTLCGSNFYLHPSGLVPEGTHQVTVGQSPCRPL
PKDSSKLRPVPRKDFVEEFECELEPLGTQAVGPTN
VSLTVTNMPPGKHFRVDGTSVLRGFSFMEPVLIAV
QPLFGPRAGGTCLTLEGQSLSVGTSRAVLVNGTEC
LLARVSEGQLLCATPPGATVASVPLSLQVGGAQVP
GSWTFQYREDPVVLSISPNCGYINSHITICGQHLT
SAWHLVLSFHDGLRAVESRCERQLPEQQLCRLPEY
VVRDPQGWVAGNLSARGDGAAGFTLPGFRFLPPPH
PPSANLVPLKPEEHAIKFEYIGLGAVADCVGINVT
VGGESCQHEFRGDMVVCPLPPSLQLGQDGAPLQVC
VDGECHILGRVVRPGPDGVPQST

TABLE 2
Summary of screening assays performed for the antibodies disclosed herein.

| | Clone name | | | | | | | |
|---|---|---|---|---|---|---|---|---|
| Assay performed | 3G4 | 3F6 | 3F8 | 6A9 | 7H11 | 9F2 | 9F6 | 10G1 |
| ELISA | +++ | +++ | +++ | +++ | +++ | +++ | ++ | +++ |
| WB- Trans | − | − | − | − | − | − | − | − |
| WB- Endo | − | − | − | − | − | − | − | − |
| Cell staining- Trans | +++ | +++ | +++ | +++ | +++ | +++ | ++ | +++ |
| Cell staining- Endo | +++ | +++ | +++ | +++ | +++ | +++ | +++ | +++ |
| | (Mem) | (Mem) | (Mem) | (Mem) | (Mem) | (Mem) | (Mem) | (Mem) |
| Cell staining- RON KO | − | − | − | − | − | − | − | − |
| FACS (T47D cells) | ++ | ++ | ++ | ++ | ++ | ++ | ++ | ++ |
| IP | +++ | +++ | +++ | +++ | +++ | +++ | +++ | +++ |

In Table 2, “Trans” stands for transfected cells, “Endo” stands for endogenous RON expressing cells, “RON KO” stands for RON knock-out cell HCT116 cells. “Mem” indicates membrane staining. “IP” stands for immunoprecipitation.

Other embodiments of the present disclosure are provided below:

1. An antigen specific binding domain which binds to RON (Macrophage stimulating protein receptor or Recepteur d'Origine Nantais), comprising a light chain variable region (VL) and a heavy chain variable region (VH), wherein the heavy chain variable region (VH) comprises Complementarity Determining Regions (CDRs) CDRH1, CDRH2, CDRH3, and the light chain variable region comprises CDRs CDRL1, CDRL2, and CDRL3; wherein:
i. CDRH1 is selected from the group consisting of SEQ ID NO: 10, 28, 46, 67, 83, 107, 124, 140, and a CDRH1 differing from any one of the same in that 1 or 2 amino acids are replaced, deleted or added;
ii. CDRH2 is selected from the group consisting of SEQ ID NO: 11, 29, 47, 68, 84, 108, 125, 141, and a CDRH2 differing from any one of the same in that 1 or 2 amino acids are replaced, deleted or added;
iii. CDRH3 is selected from the group consisting of SEQ ID NO: 12, 30, 48, 69, 85, 109, 126, 142, and a CDRH3 differing from any one of the same in that 1 or 2 amino acids are replaced, deleted or added;
iv. CDRL1 is selected from the group consisting of SEQ ID NO: 1, 19, 37, 60, 75, 98, 115, 132, and a CDRL1 differing from any one of the same in that 1 or 2 amino acids are replaced, deleted or added;
v. CDRL2 is selected from the group consisting of SEQ ID NO: 2, 20, 38, 76, 99, 116, 133, and a CDRL2 differing from any one of the same in that 1 or 2 amino acids are replaced, deleted or added; and
vi. CDRL3 is selected from the group consisting of SEQ ID NO: 3, 21, 39, 61, 77, 100, 117, 134, and a CDRL3 differing from any one of the same in that 1 or 2 amino acids are replaced, deleted or added.
   2. An antigen specific binding domain according to item 1, wherein CDRH1 is SEQ ID NO: 10 or a sequence differing therefrom in that 1 or 2 amino acids are independently replaced (with an alternative amino acid), deleted or added.
   3. An antigen specific binding domain according to any one of items 1 to 2, wherein CRDH2 is SEQ ID NO: 11 or a sequence differing therefrom in that 1 or 2 amino acids are independently replaced (with an alternative amino acid), deleted or added.
   4. An antigen specific binding domain according to any one of items 1 to 3, wherein CRDH3 is SEQ ID NO: 12 or a sequence differing therefrom in that 1 or 2 amino acids are independently replaced (with an alternative amino acid), deleted or added.
   5. An antigen specific binding domain according to any one of item 1 to 4, wherein the CDRL1 is SEQ ID NO: 1 or a sequence differing therefrom in that 1 or 2 amino acids are independently replaced (with an alternative amino acid), deleted or added.
   6. An antigen specific binding domain according to any one of item 1 to 5, wherein the CDRL2 is SEQ ID NO: 2 or a sequence differing therefrom in that 1 or 2 amino acids are independently replaced (with an alternative amino acid), deleted or added.
   7. An antigen specific binding domain according to any one of item 1 to 6, wherein the CDRL3 is SEQ ID NO: 3 or a sequence differing therefrom in that 1 or 2 amino acids are independently replaced (with an alternative amino acid), deleted or added.
   8. An antigen specific binding domain according to any one of item 1 to 7, wherein the variable heavy domain has a sequence:
as shown in SEQ ID NOs: 17, 58 or 96;
a sequence derived from SEQ ID NOs: 17, 58, or 96, wherein 1, 2, 3, 4, 5, 6, 7, 8, 9 or 10 amino acids are independently replaced (with an alternative amino acid), deleted or added; or
in an independent aspect of the disclosure there is provided a derivative of SEQ ID NOs: 17, 58, or 96 with at least 95% identity thereto.
   9. An antigen specific binding domain according to any one of item 1 to 8, wherein the variable light domain has a sequence:
as shown in SEQ ID NOs: 8 or 93;
a sequence derived from SEQ ID NO: 8 or 93, wherein 1, 2, 3, 4, 5, 6, 7, 8, 9 or 10 amino acids are independently replaced (with an alternative amino acid), deleted or added; or
in an independent aspect of the disclosure there is provided a derivative of SEQ ID NOs: 8 or 93 at least 95% identity thereto;
   10. An antigen specific binding domain according to item 1, wherein CDRH1 is SEQ ID NO: 28 or a sequence differing therefrom in that 1 or 2 amino acids are independently replaced (with an alternative amino acid), deleted or added.
   11. An antigen specific binding domain according to item 1 or 10, wherein CDRH2 is SEQ ID NO: 29 or a sequence differing therefrom in that 1 or 2 amino acids are independently replaced (with an alternative amino acid), deleted or added.
   12. An antigen specific binding domain according to any one of items 1, 10 or 11, wherein CDRH3 is SEQ ID NO: 30 or a sequence differing therefrom in that 1 or 2 amino acids are independently replaced (with an alternative amino acid), deleted or added.
   13. An antigen specific binding domain according any one of items 1 or 10 to 12, wherein CDRL1 is SEQ ID NO: 19 or a sequence differing therefrom in that 1 or 2 amino acids are independently replaced (with an alternative amino acid), deleted or added.
   14. An antigen specific binding domain according to item 1 or 10 to 13, wherein CDRL2 is SEQ ID NO: 20 or a sequence differing therefrom in that 1 or 2 amino acids are independently replaced (with an alternative amino acid), deleted or added.
   15. An antigen specific binding domain according to item 1 or items 10 to 14, wherein CDRL3 is SEQ ID NO: 21 or a sequence differing therefrom in that 1 or 2 amino acids are independently replaced (with an alternative amino acid), deleted or added.
   16. An antigen specific binding domain according to any one of items 10 to 15, wherein the heavy chain variable region has a sequence:
as shown in SEQ ID NO: 35;
a sequence derived from SEQ ID NO: 35 wherein 1, 2, 3, 4, 5, 6, 7, 8, 9 or 10 amino acids are independently replaced (with an alternative amino acid), deleted or added; or
in an independent aspect of the disclosure there is provided a derivative of SEQ ID NO; 35 at least 95% identical thereto.
   17. An antigen specific binding domain according to any one of items 10 to 16, wherein the light chain variable region has a sequence:
as shown in SEQ ID NO: 26;
a sequence derived from SEQ ID NO: 26 wherein 1, 2, 3, 4, 5, 6, 7, 8, 9 or 10 amino acids are independently replaced (with an alternative amino acid), deleted or added; or
in an independent aspect of the disclosure there is provided a derivative of SEQ ID NO; 26 at least 95% identical thereto.
   18. An antigen specific binding domain according item 1, wherein CDRH1 is SEQ ID NO: 46 or a sequence differing therefrom in that 1 or 2 amino acids are independently replaced (with an alternative amino acid), deleted or added.
   19. An antigen specific binding domain according to item 1 or 18, wherein CDRH2 is SEQ ID NO: 47 or a sequence differing therefrom in that 1 or 2 amino acids are independently replaced (with an alternative amino acid), deleted or added.
   20. An antigen specific binding domain according to any one of items 1, 18 or 19, wherein CDRH3 is SEQ ID NO: 48 or a sequence differing therefrom in that 1 or 2 amino acids are independently replaced (with an alternative amino acid), deleted or added.
   21. An antigen specific binding domain according to any one of items 1 or 18 to 20, wherein CDRL1 is SEQ ID NO: 37 or a sequence differing therefrom in that 1 or 2 amino acids are independently replaced (with an alternative amino acid), deleted or added.
   22. An antigen specific binding domain according to any one of items 1 or 18 to 21, wherein CDRL2 is SEQ ID NO: 38 or a sequence differing therefrom in that 1 or 2 amino acids are independently replaced (with an alternative amino acid), deleted or added.
   23. An antigen specific binding domain according to any one of items 1 or 18 to 22, wherein CDRL3 is SEQ ID NO: 39 or a sequence differing therefrom in that 1 or 2 amino acids are independently replaced (with an alternative amino acid), deleted or added.
   24. An antigen specific binding domain according to any one of items 18 to 23, wherein the heavy chain variable domain has a sequence:
as shown in SEQ ID NO: 53;
a sequence derived from SEQ ID NO: 53 wherein 1, 2, 3, 4, 5, 6, 7, 8, 9 or 10 amino acids are independently replaced (with an alternative amino acid), deleted or added; or
in an independent aspect of the disclosure there is provided a derivative of SEQ ID NO; 53 at least 95% identical thereto.
   25. An antigen specific binding domain according to any one of items 18 to 24, wherein the light chain variable domain has a sequence:
shown in SEQ ID NO: 54; or
a sequence derived from SEQ ID NO: 54 wherein 1, 2, 3, 4, 5, 6, 7, 8, 9 or 10 amino acids are independently replaced (with an alternative amino acid), deleted or added, or
in an independent aspect of the disclosure there is provided a derivative of SEQ ID NO; 54 at least 95% identical thereto.
   26. An antigen specific binding domain according to item 1, wherein CDRH1 is SEQ ID NO: 67 or a sequence differing therefrom in that 1 or 2 amino acids are independently replaced (with an alternative amino acid), deleted or added.
   27. An antigen specific binding domain according to items 1 or 26, wherein CDRH2 is SEQ ID NO: 68 or a sequence differing therefrom in that 1 or 2 amino acids are independently replaced (with an alternative amino acid), deleted or added.
   28. An antigen specific binding domain according to any one of items 1 to 27, wherein CDRH3 is SEQ ID NO: 69 or a sequence differing therefrom in that 1 or 2 amino acids are independently replaced (with an alternative amino acid), deleted or added.
   29. An antigen specific binding domain according to any one of items 1 or 26 to 28, wherein CDRL1 is SEQ ID NO: 60 or a sequence differing therefrom in that 1 or 2 amino acids are independently replaced (with an alternative amino acid), deleted or added.
   30. An antigen specific binding domain according to any one of items 1 or 26 to 29, wherein CDRL2 is SEQ ID NO: 20 or a sequence differing therefrom in that 1 or 2 amino acids are independently replaced (with an alternative amino acid), deleted or added.
   31. An antigen specific binding domain according to any one of items 1 or 26 to 34, wherein CDRL3 is a SEQ ID NO: 61 or a sequence differing therefrom in that 1 or 2 amino acids are independently replaced (with an alternative amino acid), deleted or added.
   32. An antigen specific binding domain according to any one of items 1 or 26 to 31, wherein the heavy chain variable domain has sequence:
shown in SEQ ID NO: 73;
a sequence derived from SEQ ID NO: 73 wherein 1, 2, 3, 4, 5, 6, 7, 8, 9 or 10 amino acids are independently replaced (with an alternative amino acid), deleted or added; or
in an independent aspect of the disclosure there is provided a derivative of SEQ ID NO: 73 at least 95% identical thereto.
   33. An antigen specific binding domain according to any one of items 1 or 26 to 32, wherein the light chain variable domain has a sequence:
show in SEQ ID NO: 65;
a sequence derived from SEQ ID NO: 65 wherein 1, 2, 3, 4, 5, 6, 7, 8, 9 or 10 amino acids are independently replaced (with an alternative amino acid), deleted or added; or
in an independent aspect of the disclosure there is provided a derivative of SEQ ID NO: 65 at least 95% identical thereto.
   34. An antigen specific binding domain according to item 1, wherein CDRH1 is SEQ ID NO: 83 or a sequence differing therefrom in that 1 or 2 amino acids are independently replaced (with an alternative amino acid), deleted or added.
   35. An antigen specific binding domain according to item 1 or 34, wherein CDRH2 is SEQ ID NO: 84 or a sequence differing therefrom in that 1 or 2 amino acids are independently replaced (with an alternative amino acid), deleted or added.
   36. An antigen specific binding domain according to any one of items 1, 34 or 35 wherein CDRH3 is SEQ ID NO: 85 or a sequence differing therefrom in that 1 or 2 amino acids are independently replaced (with an alternative amino acid), deleted or added.
   37. An antigen specific binding domain according to any one of items 1 or 34 to 36, wherein CDRL1 is SEQ ID NO: 75 or a sequence differing therefrom in that 1 or 2 amino acids are independently replaced (with an alternative amino acid), deleted or added.
   38. An antigen specific binding domain according to any one of items 1 or 34 to 37, wherein CDRL2 is SEQ ID NO: 76 or a sequence differing therefrom in that 1 or 2 amino acids are replaced (with an alternative amino acid), deleted or added.
   39. An antigen specific binding domain according to any one of items 1 or 34 to 37, wherein CDRL3 is SEQ ID NO: 77 or a sequence differing therefrom in that 1 or 2 amino acids are independently replaced (with an alternative amino acid), deleted or added.
   40. An antigen specific binding domain according to any one of items 1 or 34 to 39, wherein the heavy chain variable domain has sequence:
shown in SEQ ID NO: 90;
a sequence derived from SEQ ID NO: 79 wherein 1, 2, 3, 4, 5, 6, 7, 8, 9 or 10 amino acids are independently replaced (with an alternative amino acid), deleted or added; or
in an independent aspect of the disclosure there is provided a derivative of SEQ ID NO: 79 at least 95% identical thereto.
   41. An antigen specific binding domain according to any one of items 1 or 34 to 40, wherein the light chain variable domain has a sequence:
as show in SEQ ID NO: 81;
a sequence derived from SEQ ID NO: 81 wherein 1, 2, 3, 4, 5, 6, 7, 8, 9 or 10 amino acids are independently replaced (with an alternative amino acid), deleted or added; or
in an independent aspect of the disclosure there is provided a derivative of SEQ ID NO: 81 at least 95% identical thereto.
   42. An antigen specific binding domain according to item 1, wherein CDRH1 is SEQ ID NO: 107 or a sequence differing therefrom in that 1 or 2 amino acids are independently replaced (with an alternative amino acid), deleted or added.
   43. An antigen specific binding domain according to item 1 or 42, wherein CDRH2 is SEQ ID NO: 108 or a sequence differing therefrom in that 1 or 2 amino acids are independently replaced (with an alternative amino acid), deleted or added.
   44. An antigen specific binding domain according to item 1, 42 or 43, wherein CDRH3 is SEQ ID NO: 109 or a sequence differing therefrom in that 1 or 2 amino acids are independently replaced (with an alternative amino acid), deleted or added.
   45. An antigen specific binding domain according to any one of item 1 or 42 to 44, wherein CDRL1 is SEQ ID NO: 98 or a sequence differing therefrom in that 1 or 2 amino acids are independently replaced (with an alternative amino acid), deleted or added.
   46. An antigen specific binding domain according to any one of items 1 or 42 to 45, wherein CDRL2 is SEQ ID NO: 99 or a sequence differing therefrom in that 1 or 2 amino acids are independently replaced (with an alternative amino acid), deleted or added.
   47. An antigen specific binding domain according to any one of item 1 or 42 to 46, wherein CDRL3 is SEQ ID NO: 100 or a sequence differing therefrom in that 1 or 2 amino acids are independently replaced (with an alternative amino acid), deleted or added.
   48. An antigen specific binding domain according to any one of items 1 or 42 to 47, wherein the heavy chain variable domain has sequence:
as shown in SEQ ID NO: 113;
a sequence derived from SEQ ID NO: 113 wherein 1, 2, 3, 4, 5, 6, 7, 8, 9 or 10 amino acids are independently replaced (with an alternative amino acid), deleted or added, or
in an independent aspect of the disclosure there is provided a derivative of SEQ ID NO: 113 at least 95% identical thereto.
   49. An antigen specific binding domain according to any one of items 1 or 42 to 48, wherein the light chain variable domain has a sequence:
show in SEQ ID NO: 105;
a sequence derived from SEQ ID NO: 105 wherein 1, 2, 3, 4, 5, 6, 7, 8, 9 or 10 amino acids are independently replaced (with an alternative amino acid), deleted or added, or
in an independent aspect of the disclosure there is provided a derivative of SEQ ID NO: 98 at least 95% identical thereto.
   50. An antigen specific binding domain according to item 1, wherein CDRH1 is SEQ ID NO: 124 or a sequence differing therefrom in that 1 or 2 amino acids are independently replaced (with an alternative amino acid), deleted or added.
   51. An antigen specific binding domain according to item 1 or 50, wherein CDRH2 is SEQ ID NO: 125 or a sequence differing therefrom in that 1 or 2 amino acids are independently replaced (with an alternative amino acid), deleted or added.
   52. An antigen specific binding domain according to item 1, 50 or 51 wherein CDRH3 is SEQ ID NO: 126 or a sequence differing therefrom in that 1 or 2 amino acids are independently replaced (with an alternative amino acid), deleted or added.
   53. An antigen specific binding domain according to any one of items 1 or 50 to 52, wherein CDRL1 is SEQ ID NO: 115 or a sequence differing therefrom in that 1 or 2 amino acids are independently replaced (with an alternative amino acid), deleted or added.
   54. An antigen specific binding domain according to any one of items 1 or 50 to 53, wherein CDRL2 is SEQ ID NO: 116 or a sequence differing therefrom in that 1 or 2 amino acids are independently replaced (with an alternative amino acid), deleted or added.
   55. An antigen specific binding domain according to any one of items 1 or 50 to 54, wherein CDRL3 is SEQ ID NO: 117 or a sequence differing therefrom in that 1 or 2 amino acids are independently replaced (with an alternative amino acid), deleted or added.
   56. An antigen specific binding domain according to any one of items 1 or 50 to 55 wherein the heavy chain variable domain has sequence:
shown in SEQ ID NO: 130;
a sequence derived from SEQ ID NO: 130 wherein 1, 2, 3, 4, 5, 6, 7, 8, 9 or 10 amino acids are independently replaced (with an alternative amino acid), deleted or added; or
in an independent aspect of the disclosure there is provided a derivative of SEQ ID NO: 130 at least 95% identical thereto.
   57. An antigen specific binding domain according to any one of items 1 or 50 to 56, wherein the light chain variable domain has a sequence:
show in SEQ ID NO: 122;
a sequence derived from SEQ ID NO: 122 wherein 1, 2, 3, 4, 5, 6, 7, 8, 9 or 10 amino acids are independently replaced (with an alternative amino acid), deleted or added; or
in an independent aspect of the disclosure there is provided a derivative of SEQ ID NO: 122 at least 95% identical thereto.
   58. An antigen specific binding domain according to item 1, wherein CDRH1 is SEQ ID NO: 140 or a sequence differing therefrom in that 1 or 2 amino acids are independently replaced (with an alternative amino acid), deleted or added.
   59. An antigen specific binding domain according to items 1 or 58, wherein CDRH2 is SEQ ID NO: 141 or a sequence differing therefrom in that 1 or 2 amino acids are independently replaced (with an alternative amino acid), deleted or added.
   60. An antigen specific binding domain according to items 1, 58 or 59 wherein CDRH3 is SEQ ID NO: 142 or a sequence differing therefrom in that 1 or 2 amino acids are independently replaced (with an alternative amino acid), deleted or added.
   61. An antigen specific binding domain according to any one of items 1 or 58 to 60, wherein CDRL1 is SEQ ID NO: 132 or a sequence differing therefrom in that 1 or 2 amino acids are independently replaced (with an alternative amino acid), deleted or added.
   62. An antigen specific binding domain according to any one of items 1 or 58 to 61, wherein CDRL2 is SEQ ID NO: 133 or a sequence differing therefrom in that 1 or 2 amino acids are independently replaced (with an alternative amino acid), deleted or added.
   63. An antigen specific binding domain according to any one of items 1 or 58 to 62 wherein CDRL3 is SEQ ID NO: 134 or a sequence differing therefrom in that 1 or 2 amino acids are independently replaced (with an alternative amino acid), deleted or added.
   64. An antigen specific binding domain according to any one of item s 1 or 58 to 63, wherein the heavy chain variable domain has sequence:
as shown in SEQ ID NO: 146;
a sequence derived from SEQ ID NO: 146 wherein 1, 2, 3, 4, 5, 6, 7, 8, 9 or 10 amino acids are independently replaced (with an alternative amino acid), deleted or added; or
in an independent aspect of the disclosure there is provided a derivative of SEQ ID NO: 146 at least 95% identical thereto.
   65. An antigen specific binding domain according to any one of items 1 or 58 to 64, wherein the light chain variable domain has sequence:
as shown in SEQ ID NO: 138;
a sequence derived from SEQ ID NO: 138 wherein 1, 2, 3, 4, 5, 6, 7, 8, 9 or 10 amino acids are independently replaced (with an alternative amino acid), deleted or added, or
in an independent aspect of the disclosure there is provided a derivative of SEQ ID NO: 138 at least 95% identical thereto.
   66. A chimeric antigen receptor comprising a binding domain according to any one of items 1 to 65.
   67. A chimeric antigen receptor according to item 66, wherein the chimeric antigen receptor is expressed on a cell surface.
   68. A chimeric antigen receptor according to item 66 or 67, wherein the cell is selected from a T cell (such as a cytotoxic T cell), an NK cell and an NKT cell.
   69. A chimeric antigen receptor according to any one of items 66 to 68, which comprises an intracellular signalling domain.
   70. A chimeric antigen receptor according to item 69, wherein the intracellular signalling domain is from CD3-zeta, for example described in WO2013/040557 incorporated herein by reference.
   71. A chimeric antigen receptor according to any one of items 66 to 70, which further comprises a co-stimulator domain
   72. A chimeric antigen receptor according to item 71, wherein the co-stimulator domain is independently selected from CD28, 4-1BB or OX40, and combinations thereof, such as CD28 and 4-1BB or CD28 and OX40.
   73. A composition for parenteral administration comprising a chimeric antigen receptor according to any one of items 66 to 72.
   74. An antibody molecule comprising a binding domain according to any one of items 1 to 65.
   75. An antibody molecule according to item 74, wherein the antibody molecule is selected from a multispecific antibody (such as a bispecific antibody), a full-length antibody or an antibody binding fragment.
   76. An antibody molecule according to item 75, where the multispecific antibody is a bispecific T cell engager (BiTe®).
   77. An antibody molecule according to item 75, wherein the multispecific antibody comprises two different antigen specific binding domains.
   78. An antibody molecule according to item 77, wherein the two different antigen specific binding domains are specific for different epitopes.
   79. An antibody molecule according to item 78, comprising an antigen specific binding domain comprising CDRH1, H2 and H3 as shown in SEQ ID NO: 10, 11 and 12, respectively and CDRL1, L2 and L3 shown in SEQ ID NO: 1, 2 and 3, respectively (in particular employed in variable regions shown in SEQ ID NO: 17 and 8, 58 and 8, or 96 and 93, or a variant of the VH and/or VL domain with at least 95% identity to said sequence).
   80. An antibody molecule according to item 78 or 79, comprising an antigen specific binding domain comprising CDRH1, H2 and H3 as shown in SEQ ID NO: 28, 29 and 30, respectively and CDRL1, L2 and L3 shown in SEQ ID NO: 19, 20 and 21, respectively (in particular employed in variable regions shown in SEQ ID NO: 35 and 26, or a variant of the VH and/or VL domain with at least 95% identity to said sequence).
   81. An antibody molecule according to any one of items 78 to 80, comprising an antigen specific binding domain comprising CDRH1, H2 and H3 as shown in SEQ ID NO: 46, 47 and 48, respectively and CDRL1, L2 and L3 show in SEQ ID NO: 37, 38 and 39, respectively (in particular employed in variable regions shown in SEQ ID NO: 53 and 44, or a variant of the VH and/or VL with at least 95% identity thereto).
   82. An antibody molecule according to any one of items 78 to 81, comprising an antigen specific binding domain comprising CDRH1, H2 and H3 as shown in SEQ ID NO: 67, 68 and 69, respectively and CDRL1, L2 and L3 show in SEQ ID NO: 60, 20 and 61 (in particular employed in variable regions shown in SEQ ID NO: 73 and 65), or a variant of the VH and/or VL with at least 95% identity thereto).
   83. An antibody molecule according to any one of items 78 to 82, comprising an antigen specific binding domain comprising CDRH1, H2 and H3 as shown in SEQ ID NO: 83, 84 and 85, respectively and CDRL1, L2 and L3 show in SEQ ID NO: 75, 76 and 77 (in particular employed in variable regions shown in SEQ ID NO: 90 and 81), or a variant of the VH and/or VL with at least 95% identity thereto).
   84. An antibody molecule according to any one of items 78 to 83, comprising an antigen specific binding domain comprising CDRH1, H2 and H3 as shown in SEQ ID NO: 107, 108 and 109, respectively and CDRL1, L2 and L3 show in SEQ ID NO: 98, 99 and 100 (in particular employed in variable regions shown in SEQ ID NO: 113 and 105), or a variant of the VH and/or VL with at least 95% identity thereto).
   85. An antibody molecule according to any one of items 78 to 84, comprising an antigen specific binding domain comprising CDRH1, H2 and H3 as shown in SEQ ID NO: 124, 125 and 126, respectively and CDRL1, L2 and L3 show in SEQ ID NO: 115, 116 and 117 (in particular employed in variable regions shown in SEQ ID NO: 130 and 122), or a variant of the VH and/or VL with at least 95% identity thereto).
   86. An antibody molecule according to any one of items 78 to 85, comprising an antigen specific binding domain comprising CDRH1, H2 and H3 as shown in SEQ ID NO: 140, 141 and 142, respectively and CDRL1, L2 and L3 show in SEQ ID NO: 132, 133 and 134 (in particular employed in variable regions shown in SEQ ID NO: 146 and 138), or a variant of the VH and/or VL with at least 95% identity thereto), or a variant of the VH and/or VL with at least 95% identity thereto).
   87. An antibody molecule according to any one of items 74 to 86, wherein the antibody binding fragment is selected from a Fab, a modified Fab, a Fab′, a modified Fab′, a F(ab′)2, an Fv, ds-FV, Fab-Fv, Fab-dsFv, a single domain antibody (e.g. VH or VL or VHH), a scFv, and a ds-scFv.
   88. An antibody molecule according to any one of items 74 to 87, wherein the antibody molecule comprises an effector function.
   89. An antibody molecule according to item 88, wherein the effector function is provided by an antibody constant region or an active fragment thereof, for example a full-length IgG isotype, such as IgG1, IgG2, IgG3 or IgG4 (in particular IgG1 or IgG4).
   90. An antibody molecule according to any one of items 74 to 89, wherein the antibody molecule is conjugated to a payload.
   91. An antibody molecule according to item 90, wherein the payload is selected from a toxin, a polymer (for example synthetic or naturally occurring polymers), biologically active proteins (for example enzymes, other antibody or antibody fragments), nucleic acids and fragments thereof (for example DNA, RNA and fragments thereof) radionuclides (particularly radioiodide, radioisotopes) chelated metals, nanoparticles and reporter groups such as fluorescent or luminescent labels or compounds which may be detected by NMR or ESR spectroscopy.
   92. An antibody molecule according to item 91, wherein the toxin in selected from an auristatin (for example MMAE (monomethyl auristatin E), MMAF (monomethyl auristatin F)), pyrrolobenzodiazepine (PBD), doxorubicin, duocarmycin, a maytansinoid (for example N 2′-deacetyl-N 2′-(3-mercapto-1-oxopropyl)-maytansine (DM1), N 2′-deacetyl-N2′-(4-mercapto-1-oxopentyl)-maytansine (DM3) and N 2′-deacetyl-N 2′(4-methyl-4-mercapto-1-oxopentyl)-maytansine (DM4)), calocheamicin, dolastatin, maytansine, α-amanitin, and a tubulysin.
   93. A pharmaceutical composition comprising an antibody molecule according to any one of items 74 to 92 and an excipient, diluent and/or carrier.
   94. A pharmaceutical composition according to item 93, comprising at least two monoclonal antibody molecules in admixture, for example two RON specific antibody molecules.
   95. A pharmaceutical composition according to item 94, wherein at least one of the RON specific antibody molecules comprises an antigen specific binding domain comprising CDRH1, H2 and H3 as shown in SEQ ID NO: 10, 11 and 12, respectively and CDRL1, L2 and L3 shown in SEQ ID NO: 1, 2 and 3, respectively (in particular employed in variable regions shown in SEQ ID NO: 17 and 8, 58 and 8, or 96 and 93), or a variant of the VH and/or VL with at least 95% identity thereto).
   96. A pharmaceutical composition according to item 94 or 95, wherein at least one of the RON specific antibody molecules comprises an antigen specific binding domain comprising CDRH1, H2 and H3 as shown in SEQ ID NO: 28, 29, 30, respectively and CDRL1, L2 and L3 show in SEQ ID NO: 19, 20 and 21 (in particular employed in variable regions shown in SEQ ID NO: 35 and 26), or a variant of the VH and/or VL with at least 95% identity thereto).
   97. A pharmaceutical composition according to any one of items 94 to 96, wherein at least one of the RON specific antibody molecules comprises an antigen specific binding domain comprising CDRH1, H2 and H3 as shown in SEQ ID NO: 46, 47 and 48, respectively and CDRL1, L2 and L3 shown in SEQ ID NO: 37, 38 and 39, respectively (in particular employed in variable regions shown in SEQ ID NO: 53 and 44), or a variant of the VH and/or VL with at least 95% identity thereto).
   98. A pharmaceutical composition according to any one of items 94 to 97, wherein at least one of the RON specific antibody molecules comprises an antigen specific binding domain comprising CDRH1, H2 and H3 as shown in SEQ ID NO: 67, 68 and 69, respectively and CDRL1, L2 and L3 show in SEQ ID NO: 60, 20 and 61 (in particular employed in variable regions shown in SEQ ID NO: 73 and 65), or a variant of the VH and/or VL with at least 95% identity thereto).
   99. A pharmaceutical composition according to any one of items 94 to 98, wherein at least one of the RON specific antibody molecules comprises an antigen specific binding domain comprising CDRH1, H2 and H3 as shown in SEQ ID NO: 83, 84 and 85, respectively and CDRL1, L2 and L3 show in SEQ ID NO: 75, 76 and 77 (in particular employed in variable regions shown in SEQ ID NO: 90 and 81), or a variant of the VH and/or VL with at least 95% identity thereto).
   100. A pharmaceutical composition according to any one of items 94 to 99, wherein at least one of the RON specific antibody molecules comprises an antigen specific binding domain comprising CDRH1, H2 and H3 as shown in SEQ ID NO: 107, 108 and 109, respectively and CDRL1, L2 and L3 show in SEQ ID NO: 98, 99 and 100 (in particular employed in variable regions shown in SEQ ID NO: 113 and 105), or a variant of the VH and/or VL with at least 95% identity thereto).
   101. A pharmaceutical composition according to any one of items 94 to 100, wherein at least one of the RON specific antibody molecules comprises an antigen specific binding domain comprising CDRH1, H2 and H3 as shown in SEQ ID NO: 124, 125 and 126, respectively and CDRL1, L2 and L3 show in SEQ ID NO: 115, 116 and 117 (in particular employed in variable regions shown in SEQ ID NO: 130 and 122), or a variant of the VH and/or VL with at least 95% identity thereto).
   102. A pharmaceutical composition according to any one of items 94 to 101, wherein at least one of the RON specific antibody molecules comprises an antigen specific binding domain comprising CDRH1, H2 and H3 as shown in SEQ ID NO: 140, 141 and 142, respectively and CDRL1, L2 and L3 show in SEQ ID NO: 132, 133 and 134 (in particular employed in variable regions shown in SEQ ID NO: 146 and 138), or a variant of the VH and/or VL with at least 95% identity thereto).
   103. A pharmaceutical composition according to any one of items 101 to 105, wherein the formulation is a parenteral.
   104. A antigen specific binding domain according to any one of items 1 to 65, for use in treatment.
   105. A chimeric antigen receptor according to any one of items 66 to 72 or a pharmaceutical composition according to item 73 for use in treatment.
   106. An antibody molecule according to any one of items 86 to 100, or a composition according to any one of items 101 to 106 for use in treatment.
   107. Use of an antigen specific binding domain according to any one of items 1 to 65, in the manufacture of a medicament for the treatment of cancer).
   108. Use of a chimeric antigen receptor according to any one of items 66 to 72 or a pharmaceutical composition according to item 73 for the manufacture of a medicament for the treatment of cancer.
   109. Use of an antibody molecule according to any one of items 74 to 92, or a composition according to any one of items 93 to 103 for the manufacture of medicament for the treatment of cancer (such as a solid cancer mass and/or metastatic cancer).
   110. A method of treating a patient for cancer comprising administering a therapeutic amount of an antigen specific binding domain according to any one of items 1 to 65.
   111. A method of treating a patient for cancer comprising administering a therapeutic amount of a chimeric antigen receptor according to any one of items 66 to 72 or a composition according to item 73.
   112. A method of treating a patient for cancer comprising administering a therapeutic amount of an antibody molecule according to any one of items 74 to 92 or a composition according to any one of items 93 to 103.
   113. A method of treating a patient population with a chimeric antigen receptor as defined in any one of items 66 to 76, a composition comprising the same according to item 76, antibody molecule as defined in any one of items 74 to 92 or a composition comprising the same according to any one of items 93 to 103, wherein the population is characterised in that patients making up the population have a RON positive tumour.
   114. A method of treating a patient population according to item 113, wherein the patients making up the population have a RON positive and MET positive tumour.
   115. A method of treating a patient population according to item 113 or 114, which comprises the step of identifying the patient as in the population before treatment according to the present disclosure is administered.
   116. A method of downregulating RON expression in a patient by administering a therapeutically effective amount of a binding domain according to the present disclosure (in particular an antibody molecule as disclosed herein).
   117. An antibody molecule which cross-blocks or binds the same epitope as an antibody molecule comprising a VH of SEQ ID NO: 17, SEQ ID NO: 35, SEQ ID NO: 53, SEQ ID NO: 51, SEQ ID NO: 63, SEQ ID NO: 79, SEQ ID NO: 95, SEQ ID NO: 106, SEQ ID NO: 121, SEQ ID NO: 130, SEQ ID NO: 133 or 142.
   118. An antibody molecule, which cross-blocks or binds the same epitope as an antibody molecule comprising a VH/VL pair selected from SEQ ID NO: 8 and 25, SEQ ID NO: 16 and 25, SEQ ID NO: 34 and 43, SEQ ID NO: 51 and 54, SEQ ID NO: 63 and 71, SEQ ID NO: 79 and 87, SEQ ID NO: 95 and 98, SEQ ID NO: 106 and 114, SEQ ID NO: 121 and 130 or SEQ ID NO: 142 and 150.

In one embodiment the epitope bound by an antibody molecule of the present disclosure binds a conformational epitope.

In one embodiment the epitope bound by an antibody molecule of the present disclosure binds a linear epitope.

In one independent aspect there is provided an antibody molecule which specifically binds at least 5 amino acids, such as 6, 7, 8, 9, 10, 11, 12, 13, 14 or 15 amino acids in a peptide sequence.

In one embodiment, the antibody molecule of the present disclosure binds to the extracellular domain (ECD) of RON. Thus, in one embodiment, the target antigen of an antibody of the present disclosure is the extracellular domain of RON.

In one embodiment, the antibody molecule of the present disclosure binds to a target antigen comprising the amino acid sequence as set forth in SEQ ID NO: 149 or a sequence at least 95% identical thereto.

In one independent aspect of the present disclosure there is provided a binding domain comprises SEQ ID NO: 8 or a sequence at least 95% identical thereto, and SEQ ID NO: 17 or a sequence at least 95% identical thereto. In one embodiment the binding domain of the present disclosure comprises SEQ ID NO: 26 or a sequence at least 95% identical thereto, and SEQ ID NO: 35 or a sequence at least 95% identical thereto. In one embodiment the binding domain of the present disclosure comprises SEQ ID NO: 44 or a sequence at least 95% identical thereto, and SEQ ID NO: 53 or a sequence at least 95% identical thereto. In one embodiment the binding domain of the present disclosure comprises SEQ ID NO: 8 or a sequence at least 95% identical thereto, and SEQ ID NO: 58 or a sequence at least 95% identical thereto. In one embodiment the binding domain of the present disclosure comprises SEQ ID NO: 65 or a sequence at least 95% identical thereto, and SEQ ID NO: 73 or a sequence at least 95% identical thereto. In one embodiment the binding domain of the present disclosure comprises SEQ ID NO: 81 or a sequence at least 95% identical thereto, and SEQ ID NO: 90 or a sequence at least 95% identical thereto. In one embodiment the binding domain of the present disclosure comprises SEQ ID NO: 93 or a sequence at least 95% identical thereto, and SEQ ID NO: 96 or a sequence at least 95% identical thereto. In one embodiment the binding domain of the present disclosure comprises SEQ ID NO: 105 or a sequence at least 95% identical thereto, and SEQ ID NO: 113 or a sequence at least 95% identical thereto. In one embodiment the binding domain of the present disclosure comprises SEQ ID NO: 122 or a sequence at least 95% identical thereto, and SEQ ID NO: 130 or a sequence at least 95% identical thereto. In one embodiment the binding domain of the present disclosure comprises SEQ ID NO: 138 or a sequence at least 95% identical thereto, and SEQ ID NO: 146 or a sequence at least 95% identical thereto.

In one embodiment the binding domain according to the present disclosure, such as an antibody molecule, which is humanized

In one embodiment there is provided a polynucleotide (such as DNA) encoding a binding domain, chimeric receptor, or an antibody molecule according to the present disclosure, for example where heavy and light chains are encoded in the same polynucleotide (such as DNA) molecule or on different polynucleotide (such as DNA) molecules.

In one embodiment there is provided a vector comprising a polynucleotide (such as DNA) according to the present disclosure.

A cell comprising a polynucleotide (such as DNA) according to the present disclosure or a vector as defined herein, for example a mammalian cell. In one embodiment the cell is a host cell, simply designed for expression of the encoded protein, for example a CHO, HEK, PerC6, E. coli or similar cells. In one embodiment the cell is a therapeutic mammalian cell, which is engineered to express a binding domain according to the present disclosure, for example T cell (such as a cytotoxic T cell), an NK cell or an NKT cell.

In one embodiment, the polynucleotide disclosed herein comprises a nucleotide sequence as set forth in one or more of the following: SEQ ID NO: 9, SEQ ID NO: 18, SEQ ID NO: 27, SEQ ID NO: 36, SEQ ID NO: 45, SEQ ID NO: 54, SEQ ID NO: 55, SEQ ID NO: 59, SEQ ID NO: 66, SEQ ID NO: 74, SEQ ID NO: 82, SEQ ID NO: 91, SEQ ID NO: 94, SEQ ID NO: 97, SEQ ID NO: 106, SEQ ID NO: 114, SEQ ID NO: 123, SEQ ID NO: 131, SEQ ID NO: 139 and SEQ ID NO: 147.

In one embodiment, the polynucleotide disclosed herein comprises the nucleotide sequences as set forth in SEQ ID NOs: 9 and 18. In one embodiment, the polynucleotide comprises the nucleotide sequences as set forth in SEQ ID NOs: 27 and 36. In one embodiment, the polynucleotide comprises the nucleotide sequences as set forth in SEQ ID NOs: 45 and 54. In one embodiment, the polynucleotide comprises the nucleotide sequences as set forth in SEQ ID NOs: 55 and 59. In one embodiment, the polynucleotide comprises the nucleotide sequences as set forth in SEQ ID NOs: 66 and 74. In one embodiment, the polynucleotide comprises the nucleotide sequences as set forth in SEQ ID NOs: 82 and 91. In one embodiment, the polynucleotide comprises the nucleotide sequences as set forth in SEQ ID NOs: 94 and 97. In one embodiment, the polynucleotide comprises the nucleotide sequences as set forth in SEQ ID NOs: 106 and 114. In one embodiment, the polynucleotide comprises the nucleotide sequences as set forth in SEQ ID NOs: 123 and 131. In one embodiment, the polynucleotide comprises the nucleotide sequences as set forth in SEQ ID NOs: 139 and 147.

In one embodiment there is provide a mammalian lymphocyte cell (in particular an NKT cell or a T cell), such as cytotoxic T cell with an engineered T cell receptor, wherein said receptor comprises 6 CDRs from an antibody as disclosed herein.

Thus in one embodiment there is provided a method for the treatment of human cancer comprising administering a monoclonal antibody that binds to the RON protein; the antibody having the CDR sequences listed.

In one embodiment there is provided a cell according to the present disclosure for use in treatment, in particular for the treatment of cancer. Advantageously, RON is expressed and may be over-expressed by many cancers, in particular pancreatic and many epithelial cancers. The binding domains of the present disclosure can target the surface expressed RON. However, the data generated by the present inventions suggests that the antibody molecules comprising said binding domains are taken into the cell (expressing the RON) by a process involving active transport known as endocytosis. Once in the cells the antibody molecule may be localised in the cytoplasm or a cell nucleus. This is likely to be really beneficial in that the toxins or biological molecule conjugated to the antibody molecule may be internalized into the cancerous cell. Thus it may be possible to minimise the systemic toxicity and off target effects, for example by employing a toxin that is only activated inside the cell.

The binding domains (and antibody molecules) of the present disclosure are specific for human RON. However, they also recognise (i.e. are cross-reactive with) at least one non-human RON protein, for example cynomologus monkey RON or mouse RON. Thus, in one embodiment, the antibodies cross react with monkey and human protein. In another embodiment, the antibodies cross react with mouse and human RON. In another embodiment, the antibodies cross react with mouse, monkey and human RON. This is a very useful unexpected property as it allows easy preclinical analysis of potential toxicity to normal tissue and organs in an animal therapeutic module allowing determination of the therapeutic index. Thus cross-reactivity is beneficial because it allows preliminary toxicology and in vivo analysis to be performed to evaluate the safety of the molecule before it is administered to a human and negates for the generation of a surrogate antibody to perform such studies with.

In one embodiment the binding domains of the present disclosure, such as an antibody molecule, downregulates RON expression on a tumour cells. This may for example render the cancer more susceptible or sensitive to a cancer treatment, such as sensitising the patient to chemotherapy.

Thus in one independent aspect there is provided a method of changing the prognosis of a cancer patient comprising converting the patient from a RON positive patient population to a RON negative patient population by administering an antibody molecule treatment according to the present disclosure.

The present inventors have generated and characterized new mouse monoclonal antibodies to the cell surface expressed receptor tyrosine kinase RON. The two antibodies H and L chains have been cloned and the VH and VL regions transferred to a human IgG scaffold. The chimeric antibodies are also active against RON confirming that that VH and VL sequences are correct.

In one embodiment the binding domains of the present disclosure (including antibody molecules) are useful as a research reagent or for use in a diagnostic. Thus among the disclosed set of antibodies, some work very well as research reagents (e.g. Western blotting/ELISA pairing), some will be useful as diagnostic tools (for example for use in immunohistochemistry).

The novelty lies in the unique sequences of the antibodies and the proof of their efficacy in the xenograft models and their effectiveness in imaging of tumours in live animals

Also provided are:

121. An isolated antibody, or fragment thereof, wherein the antibody, or fragment thereof, comprises the heavy chain CDRs of CDRH1, CDRH2, and CDRH3 and/or the light chain CDRs of CDRL1, CDRL2, and CDRL3, wherein
(i) CDRH1 is selected from the group consisting of SEQ ID NO: 10, 28, 46, 67, 83, 107, 124, 140, or a CDRH1 sequence differing 1 or 2 amino acids therefrom;
(ii) CDRH2 is selected from the group consisting of SEQ ID NO: 11, 29, 47, 68, 84, 108, 125, 141, or a CDRH2 sequence differing 1 or 2 amino acids therefrom;
(iii) CDRH3 is selected from the group consisting of SEQ ID NO: 12, 30, 48, 69, 85, 109, 126, 142, or a CDRH3 sequence differing 1 or 2 amino acids therefrom;
(iv) CDRL1 is selected from the group consisting of SEQ ID NO: 1, 19, 37, 60, 75, 98, 115, 132, or a CDRL1 sequence differing 1 or 2 amino acids therefrom;
(v) CDRL2 is selected from the group consisting of SEQ ID NO: 2, 20, 38, 76, 99, 116, 133, or a CDRL2 sequence differing 1 or 2 amino acids therefrom; and
(vi) CDRL3 is selected from the group consisting of SEQ ID NO: 3, 21, 39, 61, 77, 100, 117, 134, or a CDRL2 sequence differing 1 or 2 amino acids therefrom.
   122. The antibody of item 121, wherein the heavy chain CDRs are selected from the group consisting of:
(i) a CDRH1 having the sequence SEQ ID NO: 10, a CDRH2 having the sequence SEQ ID NO: 11, and a CDRH3 having the sequence SEQ ID NO: 12, or a CDRH1, CDRH2, or CDRH3 sequence differing 1 or 2 amino acids therefrom;
(ii) a CDRH1 having the sequence SEQ ID NO: 28, a CDRH2 having the sequence SEQ ID NO: 29, and a CDRH3 having the sequence SEQ ID NO: 30; or a CDRH1, CDRH2, or CDRH3 sequence differing 1 or 2 amino acids therefrom;
(iii) a CDRH1 having the sequence SEQ ID NO: 46, a CDRH2 having the sequence SEQ ID NO: 47, and a CDRH3 having the sequence SEQ ID NO: 48; or a CDRH1, CDRH2, or CDRH3 sequence differing 1 or 2 amino acids therefrom;
(iv) a CDRH1 having the sequence SEQ ID NO: 67, a CDRH2 having the sequence SEQ ID NO: 68, and a CDRH3 having the sequence SEQ ID NO: 69; or a CDRH1, CDRH2, or CDRH3 sequence differing 1 or 2 amino acids therefrom;
(v) a CDRH1 having the sequence SEQ ID NO: 83, a CDRH2 having the sequence SEQ ID NO: 84, and a CDRH3 having the sequence SEQ ID NO: 85; or a CDRH1, CDRH2, or CDRH3 sequence differing 1 or 2 amino acids therefrom;
(vi) a CDRH1 having the sequence SEQ ID NO: 107, a CDRH2 having the sequence SEQ ID NO: 108, and a CDRH3 having the sequence SEQ ID NO: 109; or a CDRH1, CDRH2, or CDRH3 sequence differing 1 or 2 amino acids therefrom;
(vii) a CDRH1 having the sequence SEQ ID NO: 124, a CDRH2 having the sequence SEQ ID NO: 125, and a CDRH3 having the sequence SEQ ID NO: 126; or a CDRH1, CDRH2, or CDRH3 sequence differing 1 or 2 amino acids therefrom; and
(viii) a CDRH1 having the sequence SEQ ID NO: 140, a CDRH2 having the sequence SEQ ID NO: 141, and a CDRH3 having the sequence SEQ ID NO: 142; or a CDRH1, CDRH2, or CDRH3 sequence differing 1 or 2 amino acids therefrom.
   123. The isolated antibody of item 121 or 122, wherein the light chain CDRs are selected from the group consisting of:
(i) a CDRL1 having the sequence SEQ ID NO: 1, a CDRL2 having the sequence SEQ ID NO: 2, and a CDRL3 having the sequence SEQ ID NO: 3; or a CDRL1, CDRL2, or CDRL3 sequence differing 1 or 2 amino acids therefrom;
(ii) a CDRL1 having the sequence SEQ ID NO: 19, a CDRL2 having the sequence SEQ ID NO: 20, and a CDRL3 having the sequence SEQ ID NO: 21; or a CDRL1, CDRL2, or CDRL3 sequence differing 1 or 2 amino acids therefrom;
(iii) a CDRL1 having the sequence SEQ ID NO: 37, a CDRL2 having the sequence SEQ ID NO: 38, and a CDRL3 having the sequence SEQ ID NO: 39; or a CDRL1, CDRL2, or CDRL3 sequence differing 1 or 2 amino acids therefrom;
(iv) a CDRL1 having the sequence SEQ ID NO: 60, a CDRL2 having the sequence SEQ ID NO: 20, and a CDRL3 having the sequence SEQ ID NO: 61; or a CDRL1, CDRL2, or CDRL3 sequence differing 1 or 2 amino acids therefrom;
(v) a CDRL1 having the sequence SEQ ID NO: 75, a CDRL2 having the sequence SEQ ID NO: 76, and a CDRL3 having the sequence SEQ ID NO: 77; or a CDRL1, CDRL2, or CDRL3 sequence differing 1 or 2 amino acids therefrom;
(vi) a CDRL1 having the sequence SEQ ID NO: 98, a CDRL2 having the sequence SEQ ID NO: 99, and a CDRL3 having the sequence SEQ ID NO: 100; or a CDRL1, CDRL2, or CDRL3 sequence differing 1 or 2 amino acids therefrom;
(vii) a CDRL1 having the sequence SEQ ID NO: 115, a CDRL2 having the sequence SEQ ID NO: 116, and a CDRL3 having the sequence SEQ ID NO:117; or a CDRL1, CDRL2, or CDRL3 sequence differing 1 or 2 amino acids therefrom; and
(viii) a CDRL1 having the sequence SEQ ID NO: 132, a CDRL2 having the sequence SEQ ID NO: 133, and a CDRL3 having the sequence SEQ ID NO:134; or a CDRL1, CDRL2, or CDRL3 sequence differing 1 or 2 amino acids therefrom.
   124. The isolated antibody of items 122 or 123, wherein the heavy chain CDRs and light chain CDRs are selected from the group consisting of:
(i) a CDRH1 having the sequence SEQ ID NO: 10, a CDRH2 having the sequence SEQ ID NO: 11, and a CDRH3 having the sequence SEQ ID NO: 12, and a CDRL1 having the sequence SEQ ID NO: 1, a CDRL2 having the sequence SEQ ID NO: 2, and a CDRL3 having the sequence SEQ ID NO: 3;
(ii) a CDRH1 having the sequence SEQ ID NO: 28, a CDRH2 having the sequence SEQ ID NO: 29, and a CDRH3 having the sequence SEQ ID NO: 30, and a CDRL1 having the sequence SEQ ID NO: 19, a CDRL2 having the sequence SEQ ID NO: 20, and a CDRL3 having the sequence SEQ ID NO: 21;
(iii) a CDRH1 having the sequence SEQ ID NO: 46, a CDRH2 having the sequence SEQ ID NO: 47, and a CDRH3 having the sequence SEQ ID NO: 48, and a CDRL1 having the sequence SEQ ID NO: 37, a CDRL2 having the sequence SEQ ID NO: 38, and a CDRL3 having the sequence SEQ ID NO: 39;
(iv) a CDRH1 having the sequence SEQ ID NO: 67, a CDRH2 having the sequence SEQ ID NO: 68, and a CDRH3 having the sequence SEQ ID NO: 69, and a CDRL1 having the sequence SEQ ID NO: 60, a CDRL2 having the sequence SEQ ID NO: 20, and a CDRL3 having the sequence SEQ ID NO: 61;
(v) a CDRH1 having the sequence SEQ ID NO: 83, a CDRH2 having the sequence SEQ ID NO: 84, and a CDRH3 having the sequence SEQ ID NO: 85, and a CDRL1 having the sequence SEQ ID NO: 75, a CDRL2 having the sequence SEQ ID NO: 76, and a CDRL3 having the sequence SEQ ID NO: 77;
(vi) a CDRH1 having the sequence SEQ ID NO: 107, a CDRH2 having the sequence SEQ ID NO: 108, and a CDRH3 having the sequence SEQ ID NO: 109, and a CDRL1 having the sequence SEQ ID NO: 98, a CDRL2 having the sequence SEQ ID NO: 99, and a CDRL3 having the sequence SEQ ID NO:100;
(vii) a CDRH1 having the sequence SEQ ID NO: 124, a CDRH2 having the sequence SEQ ID NO: 125, and a CDRH3 having the sequence SEQ ID NO: 126, and a CDRL1 having the sequence SEQ ID NO: 115, a CDRL2 having the sequence SEQ ID NO: 116, and a CDRL3 having the sequence SEQ ID NO: 117; and
(viii) a CDRH1 having the sequence SEQ ID NO: 140, a CDRH2 having the sequence SEQ ID NO: 141, and a CDRH3 having the sequence SEQ ID NO: 142, and a CDRL1 having the sequence SEQ ID NO: 132, a CDRL2 having the sequence SEQ ID NO: 133, and a CDRL3 having the sequence SEQ ID NO: 134.
   125. The isolated antibody of any one of the preceding items, wherein the antibody comprises a heavy chain variable region encoded by a nucleotide sequence having at least 80%, or at least 85% or at least 90%, or at least 95%, or at least 96%, or at least 97%, or at least 98%, or at least 99% sequence identity to one of the sequences selected from the group consisting of SEQ ID NO: 18, SEQ ID NO: 36, SEQ ID NO:54, SEQ ID NO: 59, SEQ ID NO: 74, SEQ ID NO: 91, SEQ ID NO: 97, SEQ ID NO: 114, SEQ ID NO: 131 and SEQ ID NO: 117.
   126. The isolated antibody of any one of the preceding items, wherein the antibody comprises a light chain variable region encoded by a nucleotide sequence having at least 80%, or at least 85% or at least 90%, or at least 95%, or at least 96%, or at least 97%, or at least 98%, or at least 99% sequence identity to one of selected from the group consisting of SEQ ID NO: 9, SEQ ID NO: 27, SEQ ID NO: 45, SEQ ID NO: 55, SEQ ID NO: 66, SEQ ID NO: 82, SEQ ID NO: 94, SEQ ID NO: 106, SEQ ID NO: 123, and SEQ ID NO: 139.
   127. The isolated antibody of any one of the preceding items, wherein the antibody comprises a heavy chain variable region encoded by a nucleotide sequence to one of selected from the group consisting of SEQ ID NO: 18, SEQ ID NO: 36, SEQ ID NO:54, SEQ ID NO: 59, SEQ ID NO: 74, SEQ ID NO: 91, SEQ ID NO: 97, SEQ ID NO: 114, SEQ ID NO: 131 and SEQ ID NO: 117.
   128. The isolated antibody of any one of the preceding items, wherein the antibody comprises a light chain variable region encoded by a nucleotide sequence to one of selected from the group consisting of SEQ ID NO: 9, SEQ ID NO: 27, SEQ ID NO: 45, SEQ ID NO: 55, SEQ ID NO: 66, SEQ ID NO: 82, SEQ ID NO: 94, SEQ ID NO: 106, SEQ ID NO: 123, and SEQ ID NO: 139.
   129. The isolated antibody of any one of the preceding items, wherein the antibody comprises a heavy chain variable region selected from the group consisting of:
(i) an amino acid sequence of SEQ ID NO: 17, SEQ ID NO:58, or SEQ ID NO: 96;
(ii) an amino acid sequence SEQ ID NO: 35;
(iii) an amino acid sequence SEQ ID NO: 53;
(iv) an amino acid sequence SEQ ID NO: 58;
(v) an amino acid sequence SEQ ID NO: 73;
(vi) an amino acid sequence SEQ ID NO: 90;
(vii) an amino acid sequence SEQ ID NO: 96;
(viii) an amino acid sequence SEQ ID NO: 130;
(ix) an amino acid sequence SEQ ID NO: 146; and
(x) an amino acid sequence having a sequence identity of at least 85%, or at least 90%, or at least 95%, or at least 96%, or at least 97%, or at least 98%, or at least 99%, or at least 99.95%, or 100% of (i) to (ix).
   130. The isolated antibody of any one of the preceding items, wherein the antibody comprises a light chain variable region selected from the group consisting of:
(i) an amino acid sequence of SEQ ID NO: 8;
(ii) an amino acid sequence SEQ ID NO: 26;
(iii) an amino acid sequence SEQ ID NO: 44;
(iv) an amino acid sequence SEQ ID NO: 65;
(v) an amino acid sequence SEQ ID NO: 81;
(vi) an amino acid sequence SEQ ID NO: 93;
(vii) an amino acid sequence SEQ ID NO: 105;
(viii) an amino acid sequence SEQ ID NO: 122;
(ix) an amino acid sequence SEQ ID NO: 138; and
(viii) an amino acid sequence having a sequence identity of at least 85%, or at least 90%, or at least 95%, or at least 96%, or at least 97%, or at least 98%, or at least 99%, or at least 99.95%, or 100% of (i) to (ix).
   131. The isolated antibody of any one of the preceding items, further comprising a framework sequence.
   132. The isolated antibody of item 131, wherein the heavy chain framework sequence comprises at least 90%, or at least 95%, or at least 96%, or at least 97%, or at least 98%, or at least 99%, or 100% sequence identity to framework selected from the group consisting of:
(i) an FR1 having the sequence SEQ ID NO: 13, an FR2 having the sequence SEQ ID NO: 14, an FR3 having the sequence SEQ ID NO: 15, and an FR4 having the sequence SEQ ID NO: 16; or
an FR1 having the sequence of SEQ ID NO: 56, an FR2 having the sequence SEQ ID NO: 57, an FR3 having the sequence SEQ ID NO: 15, and an FR4 having the sequence SEQ ID NO: 52; or
an FR1 having the sequence of SEQ ID NO: 95, an FR2 having the sequence SEQ ID NO: 14, an FR3 having the sequence SEQ ID NO: 15, and an FR4 having the sequence SEQ ID NO: 52;
(ii) an FR1 having the sequence SEQ ID NO: 31, an FR2 having the sequence SEQ ID NO: 32, an FR3 having the sequence SEQ ID NO: 33, and an FR4 having the sequence SEQ ID NO: 34;
(iii) an FR1 having the sequence SEQ ID NO: 46, an FR2 having the sequence SEQ ID NO: 47, an FR3 having the sequence SEQ ID NO: 48, and an FR4 having the sequence SEQ ID NO: 49;
(iv) an FR1 having the sequence SEQ ID NO:70, an FR2 having the sequence SEQ ID NO: 71, an FR3 having the sequence SEQ ID NO: 72, and an FR4 having the sequence SEQ ID NO: 52;
(v) an FR1 having the sequence SEQ ID NO: 86, an FR2 having the sequence SEQ ID NO: 87, an FR3 having the sequence SEQ ID NO: 88, and an FR4 having the sequence SEQ ID NO: 89;
(vi) an FR1 having the sequence SEQ ID NO: 110, an FR2 having the sequence SEQ ID NO: 111, an FR3 having the sequence SEQ ID NO: 112, and an FR4 having the sequence SEQ ID NO: 89;
(vii) an FR1 having the sequence SEQ ID NO: 127, an FR2 having the sequence SEQ ID NO: 128, an FR3 having the sequence SEQ ID NO: 129, and an FR4 having the sequence SEQ ID NO: 34; and
(viii) an FR1 having the sequence SEQ ID NO: 143, an FR2 having the sequence SEQ ID NO: 144, an FR3 having the sequence SEQ ID NO: 145, and an FR4 having the sequence SEQ ID NO: 34.
   133. The isolated antibody of item 131 or 132, wherein the light chain framework sequence comprises at least 90%, or at least 95%, or at least 96%, or at least 97%, or at least 98%, or at least 99%, or 100% sequence identity to framework selected from the group consisting of:
(i) an FR1 having the sequence SEQ ID NO: 4, an FR2 having the sequence SEQ ID NO: 5, an FR3 having the sequence SEQ ID NO: 6, and an FR4 having the sequence SEQ ID NO: 7; or an FR1 having the sequence SEQ ID NO: 92, an FR2 having the sequence SEQ ID NO: 5, an FR3 having the sequence SEQ ID NO: 6, and an FR4 having the sequence SEQ ID NO: 7;
(ii) an FR1 having the sequence SEQ ID NO: 22, an FR2 having the sequence SEQ ID NO: 23, an FR3 having the sequence SEQ ID NO: 24, and an FR4 having the sequence SEQ ID NO: 25;
(iii) an FR1 having the sequence SEQ ID NO: 40, an FR2 having the sequence SEQ ID NO: 41, an FR3 having the sequence SEQ ID NO: 42, and an FR4 having the sequence SEQ ID NO: 43;
(iv) an FR1 having the sequence SEQ ID NO: 62, an FR2 having the sequence SEQ ID NO: 63, an FR3 having the sequence SEQ ID NO: 64, and an FR4 having the sequence SEQ ID NO: 25;
(v) an FR1 having the sequence SEQ ID NO: 78, an FR2 having the sequence SEQ ID NO: 79, an FR3 having the sequence SEQ ID NO: 80, and an FR4 having the sequence SEQ ID NO:7;
(vi) an FR1 having the sequence SEQ ID NO: 92, an FR2 having the sequence SEQ ID NO: 5, an FR3 having the sequence SEQ ID NO: 6, and an FR4 having the sequence SEQ ID NO: 7;
(vii) an FR1 having the sequence SEQ ID NO: 101, an FR2 having the sequence SEQ ID NO: 102, and an FR3 having the sequence SEQ ID NO: 103, and an FR4 having the sequence SEQ ID NO: 104;
(viii) an FR1 having the sequence SEQ ID NO: 118, an FR2 having the sequence SEQ ID NO: 119, an FR3 having the sequence SEQ ID NO: 120, and an FR4 having the sequence SEQ ID NO: 121; and
(ix) an FR1 having the sequence SEQ ID NO: 135, an FR2 having the sequence SEQ ID NO: 136, and an FR3 having the sequence SEQ ID NO: 137, and an FR4 having the sequence SEQ ID NO: 121.
   134. The isolated antibody of any one of the preceding items, wherein the antibody, or fragment thereof, binds to a receptor tyrosine kinase RON (Recepteur d'Origine Nantais (RON), or Macrophage Stimulating Protein Receptor (MSP R, or MST1-R)).
   135. The isolated antibody of any one of the preceding items, wherein the antibody binds specifically to a human and/or a mouse and/or monkey RON.
   136. The isolated antibody of any of the preceding items, wherein the antibody binds to a cell surface expressed RON.
   137. The isolated antibody of any one of the preceding items, wherein the antibody is capable of being (or is) endocytosed by a target cell. This may be particular advantageous when the antibody molecule is conjugated to a payload because the payload may be delivered into the cell (not just to the exterior of the cell). Thus the use of payloads that are activated by enzymes inside the cancer cell are envisaged.
   138. The isolated antibody of any one of the preceding items, wherein the antibody is one of selected from the group consisting of a monoclonal antibody.
   139. The isolated antibody of any one of the preceding items, wherein the antibody is a chimeric antibody and/or multispecific antibody.
   140. The isolated antibody of any one of the preceding items, wherein the antibody is a humanized antibody.
   141. The isolated antibody of any one of the preceding items, wherein the fragment is selected from the group consisting of a Fab fragment, a Fab′ fragment, a F(ab′)2 fragment, a Fv fragment, a diabody, and a single chain antibody molecule.
   142. The isolated antibody of any one of the preceding items, wherein the antibody is an IgG1 antibody, an IgG2 antibody, an IgG3 antibody, or an IgG4 antibody.
   143. The isolated antibody of any one of the preceding items, wherein the antibody is transferred to a human IgG scaffold.
   144. The isolated antibody of any one of the preceding items, wherein the antibody is conjugated to a chemical moiety selected from the group consisting of a therapeutic agent, an immunoadhesion molecule, and a detection label.
   145. The isolated antibody of item 144, wherein the detection label is selected from the group consisting of a radiolabel, an enzyme, a fluorescent label, a luminescent label, a bioluminescent label, a magnetic label and a biotin.
   146. The isolated antibody of item 145, wherein the therapeutic agent is an anti-cancer treatment.
   147. A pharmaceutical composition comprising the antibody or fragment thereof of any one of the preceding items.
   148. The pharmaceutical composition of item 147 further comprising a pharmaceutically acceptable carrier, excipient, and/or stabilizer.
   149. A kit, comprising the isolated antibody of any one of item 121 to 146.
   150. An isolated nucleic acid comprising any one selected from the group consisting of:
(a) a nucleotide sequence encoding the antibody or fragment thereof of any one of items 121 to 146;
(b) a nucleotide sequence encoding:
   (i) a heavy chain region having at least 80%, or at least 90%, or at least 95%, or at least 96%, or at least 97%, or at least 98%, or at least 99%, or 100% of one selected from the group consisting of SEQ ID NO: 18, SEQ ID NO: 36, SEQ ID NO:54, SEQ ID NO: 59, SEQ ID NO: 74, SEQ ID NO: 91, SEQ ID NO: 97, SEQ ID NO: 114, SEQ ID NO: 131 and SEQ ID NO: 117; and
   (ii) a light chain region having at least 80%, or at least 90%, or at least 95%, or at least 96%, or at least 97%, or at least 98%, or at least 99%, or 100% of one selected from the group consisting of SEQ ID NO: 9, SEQ ID NO: 27, SEQ ID NO: 45, SEQ ID NO: 55, SEQ ID NO: 66, SEQ ID NO: 82, SEQ ID NO: 94, SEQ ID NO: 106, SEQ ID NO: 123, and SEQ ID NO: 139.
(c) a nucleic acid complementary to any one of the sequences in (a) or (b); and
(d) a nucleic acid sequence capable of hybridizing to (a), (b), or (c) under stringent conditions.
   151. An expression vector comprising the nucleic acid of item 150.
   152. A host cell comprising the expression vector of item 151.
   153. A method of producing a polypeptide comprising an immunoglobulin heavy chain variable region and/or an immunoglobulin light chain variable region, the method comprising:
(a) growing the host cell of item 152 under conditions so that the host cell expresses the polypeptide comprising the immunoglobulin heavy chain variable region and/or the immunoglobulin heavy chain variable region.
   154. The method of item 153, wherein the method further comprises the step of:
(b) purifying the polypeptide comprising the immunoglobulin heavy chain variable region and/or the immunoglobulin light chain variable region.
   155. An isolated antibody or fragment thereof of items 121 to 146, or pharmaceutical composition of item 147 or 148, for use in medicine.
   156. An isolated antibody or fragment thereof of items 121 to 146, or pharmaceutical composition of item 147 or 148, for use in treating cancer.
   157. A method of treating cancer in a subject in need thereof, comprising administering an isolated antibody, or fragment thereof, of items 121 to 146 to the subject.
   158. The method of item 157, further comprising administering a therapeutic agent selected from the group consisting of an anti-cancer agent (such as a second anti-cancer antibody), and a chemotherapeutic agent.
   159. The method of item 158, wherein the anti-cancer agent is a drug conjugate (such as mertansine or DM1 (N2′-deacetyl-N2′-(3-mercapto-1-oxopropyl)-maytansine).
   160. The method of any one of items 157 to 159, wherein the cancer is a RON expressing cancer (such as, but is not limited to colorectal cancer, non-small cell lung cancer, breast cancer, ovarian cancer, prostate cancer, cervical cancer, lung cancer, renal cancer, bladder cancer, gastrointestinal tumors, liver cancer, pancreatic cancer, gastric cancer, and head and neck cancers).
   161. The method of any one of items 157 to 160, wherein the isolated antibody or fragment thereof, is administered in a pharmaceutically effective amount.
   162. A method of detecting a tumor cell in a subject, comprising detecting the expression of RON in a sample obtained from the subject using an antibody according to any one of items 121 to 146. 163. The method of item 162, wherein the tumor cell is a cancer cell.
   164. The method of item 163, wherein the cancer is a RON expressing cancer (such as, but is not limited to colorectal cancer, non-small cell lung cancer, breast cancer, ovarian cancer, prostate cancer, cervical cancer, lung cancer, renal cancer, bladder cancer, gastrointestinal tumors, liver cancer, pancreatic cancer, gastric cancer, and head and neck cancers).

### EXAMPLES

### Example 1: Immunization of Full Length RON ECD (Extracellular Domain) into Mice

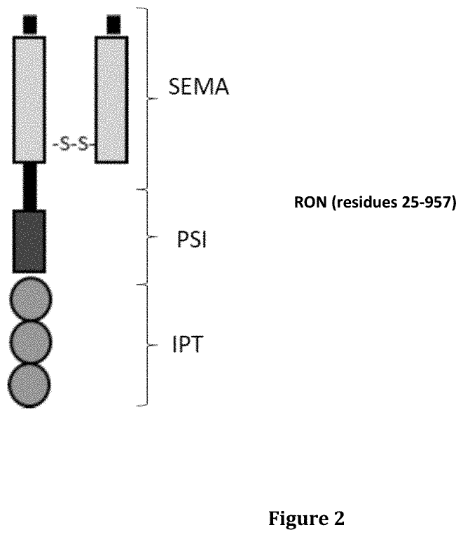

ExpiCHO cells were used for expression of extracellular human RON protein from amino acids Gly25 to Thr957 fused to an N-terminal His-tag. The expressed protein was purified by Nickel affinity purification using fast purification liquid chromatography (FPLC) and used as an immunogen, following an optimized mouse immunization schedule. Five 8 weeks old Balb/c female mice were obtained from Biological Resource Center (Singapore) and inoculated with the full-length RON extracellular domain (ECD), which corresponds to residues 25 to 957 of the entire RON protein, with a C-terminal 6×HIS tag. See FIG. 2. The mice were immunised by subcutaneous injections using the antigen mixed with Sigma Adjuvant System (Sigma) as adjuvant (1:1 ratio), at 3-week intervals.

### Example 2: Prescreening—1^{st} Bleed

One week after the third immunization, blood was drawn from each mouse via cheek bleed using a lancet (MEDIpoint International Inc.). Approximately 10 μl of blood was centrifuged for 10 min at 1600 rpm and serum was aspirated and stored at 4° C. RON ECD protein and a protein expressing HIS tag (control protein) was coated in individual wells and tested for their reactivity with serum collected from individual mouse by enzyme-linked immunosorbent assay (ELISA). Serum was diluted 10× from 1 in 1000. A 2^{nd} sera collection was also performed, this time one week after the 4^{th} immunization.

### Example 3: Fusion of Mice Spleen with Myeloma Cells to Obtain Hybridoma Cells

The mice received a final boost by injection of the RON ECD antigen without adjuvant. The mouse with the highest serum antibody titer, was selected as the spleen donor for fusion with myeloma cell line SP2/0. Thus, one week before fusion, cells were cultured in RPMI (Gibco) and 10% FBS until they attained >70% confluency in the logarithmic phase. The spleen cells of the immunised mouse were removed under sterile conditions. Generation, selection and cloning of hybridoma cells were performed using the ClonaCell-HY Hybridoma Cloning kit (STEMCELL Technologies) following the manufacturer's protocol.

ELISA screening was performed for 1600 hybridoma clones picked. From the hybridoma clones, 12 antibodies were identified to have comparable affinity for the RON ECD antigen as the polyclonal mouse serum control (O.D. 650 nm of 0.458), and are completely non-cross reactive to the control protein. The control protein used was an irrelevant protein expressing HIS tag.

### Example 4: Screening of Hybridoma Cells for RON Antibodies

Hybridoma clones secreting mAbs targeting human RON were selected by ELISA assay with the use of 96-well Maxisorp plates (Nunc) coated separately with RON ECD and His tag protein. Supernatant collected from individual hybridoma wells were tested on ELISA plates. 10% fetal bovine serum (FBS) was used for blocking and antibody dilution. 1×PBS with 0.05% Tween 20 (PBST) was used for washes. After washing, IgGs were detected using 1:5000 goat anti-mouse IgG conjugated to HRP (Biorad) in PBST with 10% FBS. After washing, plates were developed with 1×TMB ELISA substrate solution (Sigma). Absorbance was measured at 650 nm with EnVision Plate Reader (Perkin Elmer).

### Example 5: AlphaLISA SureFire Ultra p-ERK1/2 (Thr202/Tyr204) Assay

To assess the ability of the anti-RON antibodies to inhibit downstream Ras/Raf/MEK/ERK signalling pathway, an AlphaLISA SureFire Ultra p-ERK1/2 assay (Perkin Elmer) was performed.\

he T47D cell line shows MSP-dependent p-ERK expression and was therefore selected for use in the AlphaLISA assays. T47D cells seeded into 96 well plate and allowed to adhere overnight on Day 1. Next, the T47D cells were serum starved overnight on Day 2. The following day, cells were treated with the anti-RON antibodies (10 ug/ml) for an hour. As positive controls, the cells were also treated with the PI3K inhibitor wortmannin (250 nM) and immunized mouse serum (1:500). Next, the cells were stimulated with or without MSP (400 ng/ml) for 30 mins before harvesting for AlphaLISA assay. The experiments were run in duplicates.

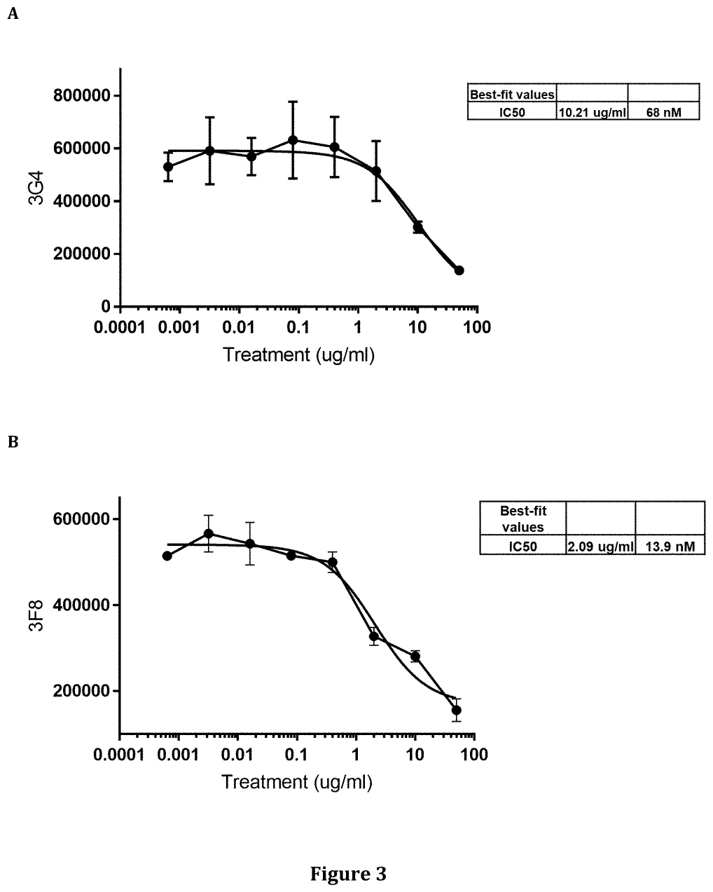

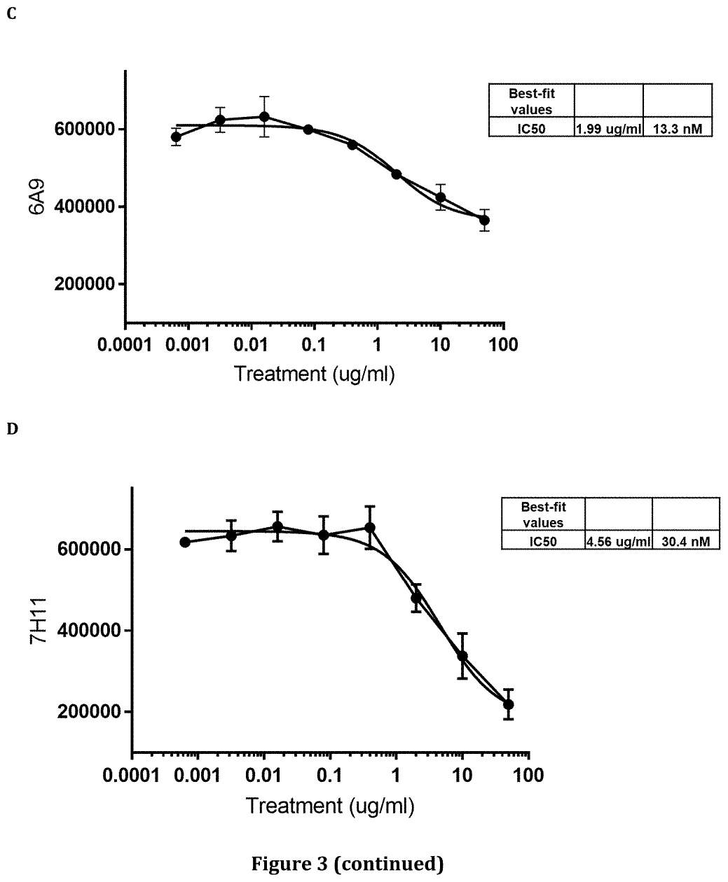

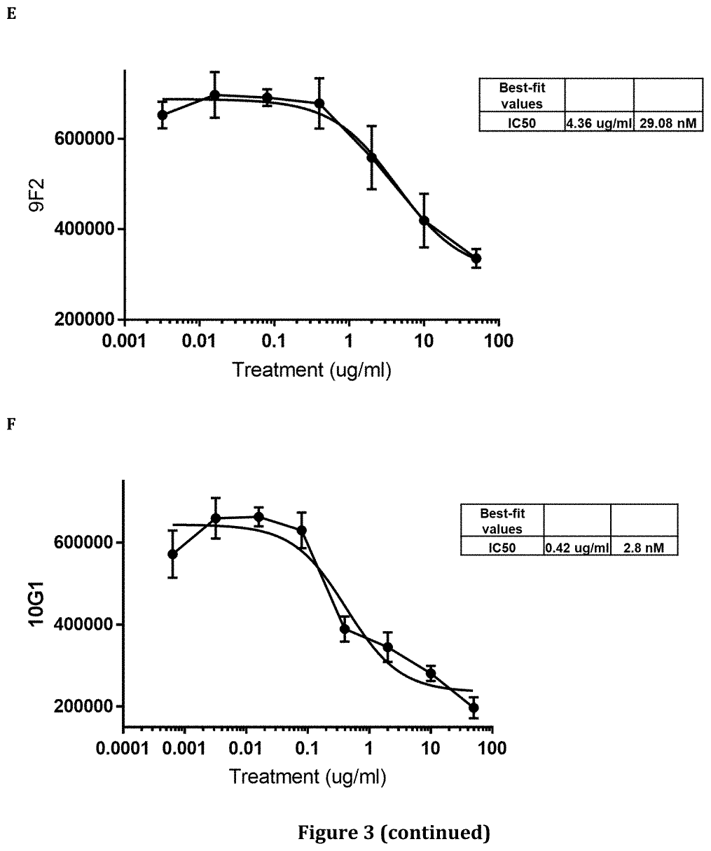

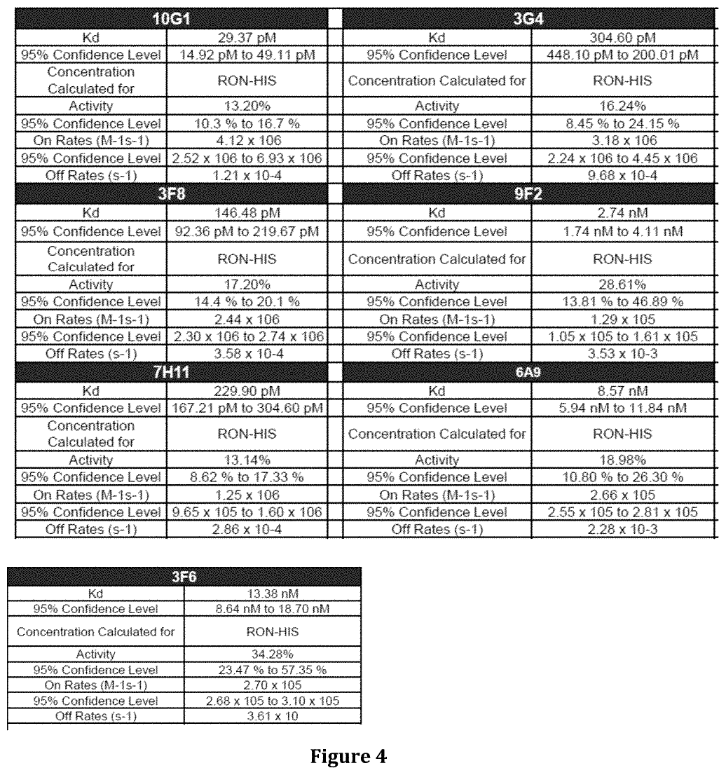

FIG. 3 is a summary of results obtained for the anti-RON antibodies vs the Wortmannin and mouse serum controls. FIGS. 4A to F shows the detailed results for the anti-RON antibodies of the present disclosure.

As can be seen from FIG. 3, the anti-RON antibodies were able to significantly reduce the alpha signal for T47D cells that were treated with MSP. In particular, there was very little difference in the alpha signal measured for the T47D cells that were treated with antibody 10G1 with or without MSP. This suggests that antibody 10G1 was able to almost completely suppress ERK activation in the T47D cells.

### Example 6—Affinity Measurement of Anti-RON Antibodies

The KinExA assay is used to measure binding affinities of tight binders (at least nanomolar range) and is able to measure the free concentration of either the receptor or the ligand without perturbing the equilibrium. Sample of receptor and ligand is prepared and the free fraction is repeatedly measured over time as it approaches equilibrium, this allows the on rate (kₒₙ) to be calculated from the curve.

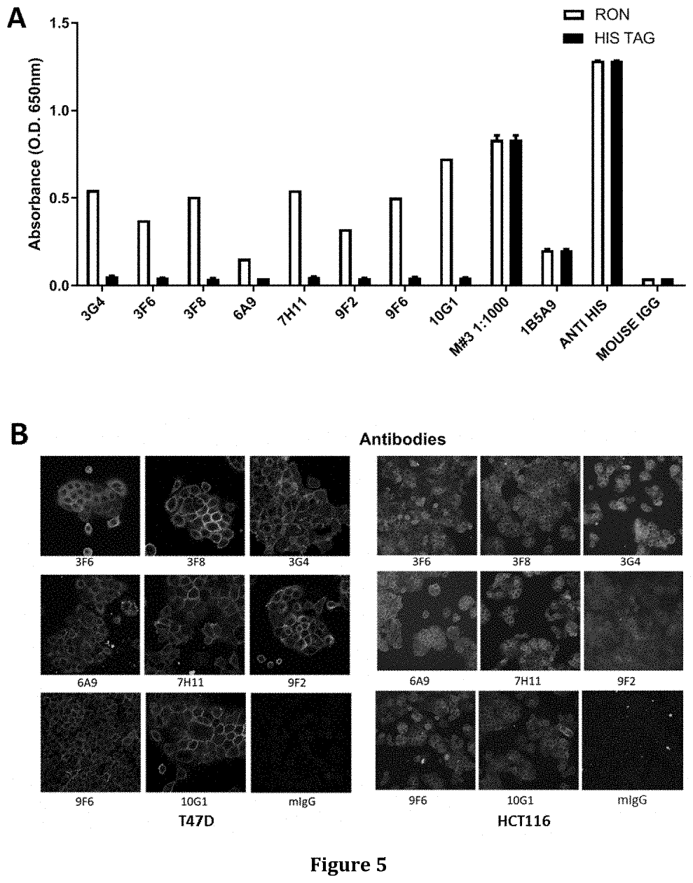

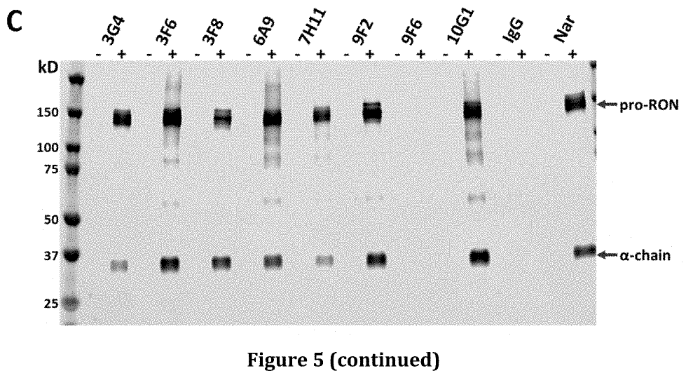

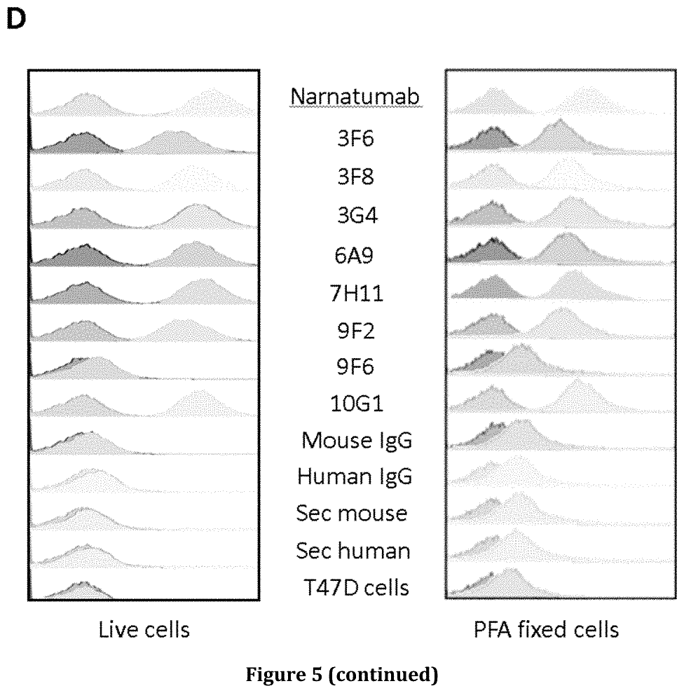

Using the KinExA assay, the binding affinities of the anti-RON antibodies for RON were determined. The results are shown in FIG. 5.

In summary, this example demonstrates that the anti-RON antibodies of the present disclosure have high binding affinities to RON and are able to significantly inhibit MSP/RON-dependent downstream Ras/Raf/MEK/ERK signalling pathway.

### Example 7—Screening and Characterization of Antibodies Targeting Membrane Bound RON

We set out to make a panel of anti-RON monoclonal antibodies using the mammalian produced His-tagged RON preparation as the immunogen. The immunogen is the ECD of RON made in, and purified from, mammalian cells. This protein extended from amino acid Gly25 to Thr 957 and contained a N terminal His-tag. In order to select for novel antibodies, we used the immunogen in an ELISA assay to screen the mice after immunization and to screen the hybridoma fusion and supplemented this assay with the immunofluorescent staining of two human cancer cell lines that express RON (HCT116 and T47D) in a high throughput 96 well based assay using a Incell Analyzer (GE Healthcare). The CRISPR technique was used to “knock-out” the RON gene from three cell lines (HCT116, HT29 and T47D cells) to use as controls in counter screening assays. Mice were immunized with the mammalian produced RON protein in adjuvant and mouse sera from one of the animals showed a high titre in the ELISA assay (FIG. 5A). In this assay we compared binding to the His-tagged RON protein to binding to another His-tagged protein. While the commercial anti-RON antibody and the mouse serum from our immunized donor mouse showed binding to both proteins as did a commercial antibody to the His-tag, the eight selected new mouse monoclonal antibodies were shown to bind strongly to the mammalian cell produced His-tagged RON but not to the control His-tagged protein. We next examined the binding of the antibodies to the cell surface of live unfixed human cancer cell lines T47D and HCT116. To block receptor mediated endocytosis, we fixed the cells with 4% paraformaldehyde after the primary antibody addition and wash step before detecting bound antibody with an Alex Fluor conjugated anti mouse IgG antibody. All of the eight monoclonal antibodies showed strong cell surface staining by confocal microscopy, while a mouse IgG control gave only a weak background stain (FIG. 5B). As an additional control we used the CRISPR RON knockout cells in the same protocol and showed that none of the new antibodies could stain these cells providing strong evidence that the antibodies were RON specific and devoid of non-specific cell surface binding. We next tested the antibodies for their ability to immunoprecipitate the RON protein from cell extracts. Extracts from T47D cells and T47D RON knock out cells were prepared using RIPA buffer and the after overnight incubation with the monoclonal antibody the immune complex was collected using protein G magnetic dynabeads. After washing the samples were eluted in SDS sample buffer separated by SDS polyacrylamide gel electrophoresis and transferred on a nitrocellulose membrane. To probe the blot, we used the anti-RON alpha chain monoclonal antibody 6E6 (WO 2018/038684) directly conjugated to peroxidase This method gave extremely clean results. The control anti-RON antibody, Narnatumab, immunoprecipitated both the free alpha chain of RON at 35 Kda and uncleaved pro-RON at 150 KDa respectively. As expected, nothing was detected in the RON knock-out cell immune precipitates (FIG. 5C). Our new monoclonal antibodies showed strong and specific immunoprecipitation of the pro-RON and the RON alpha chain species. Finally, we tested the antibodies ability to bind to cell surface RON using flow cytometry. The antibody 6E6 only bound to the cell surface after mild fixation with paraformaldehyde but could not bind to unfixed live cells. We therefore compared binding of this panel of antibodies to both live cells and PFA fixed cells. In the live cell assay Narnatumab and seven of the eight new antibodies showed a clear completely right shifted population of strongly positive T47D cells (lighter peak) but not RON knockout T47D cells (lighter peak). It was also not seen with the new antibody 9F6 nor with any of the control antibodies. Upon PFA treatment, all of the Narnatumab stained cells were right shifted apart from 9F6 which did not show binding. When we used the same reagents to stain the T47D knock out cells all right shifted peaks were lost suggesting that the staining seen on both live and PFA fixed cells is dependent on the expression of the RON protein (FIG. 5D).

### Example 8—Determination of the Binding Affinities of the Antibodies by KinEXa

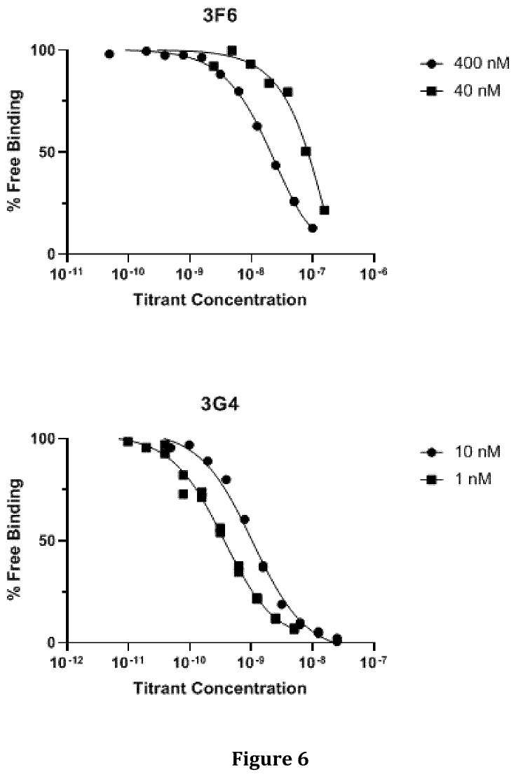

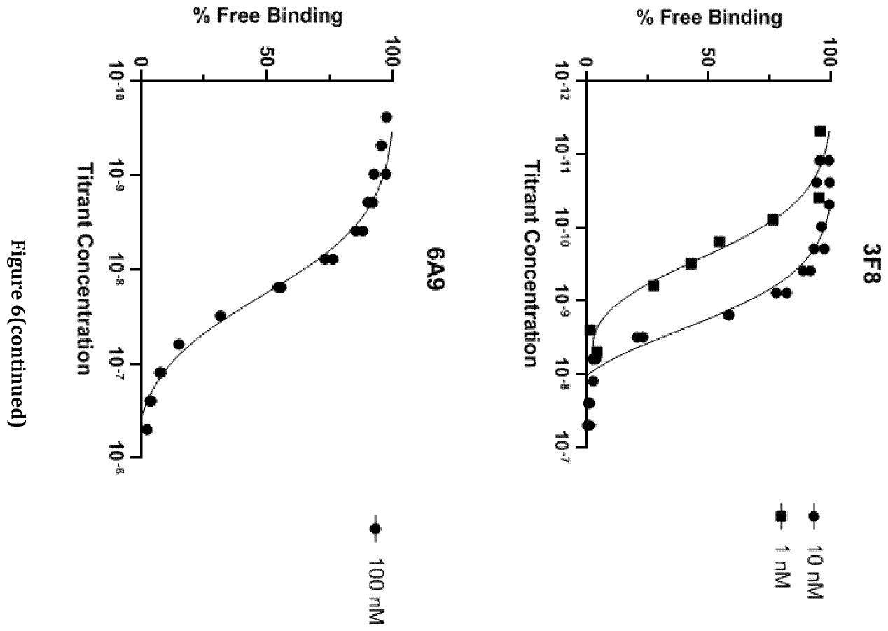

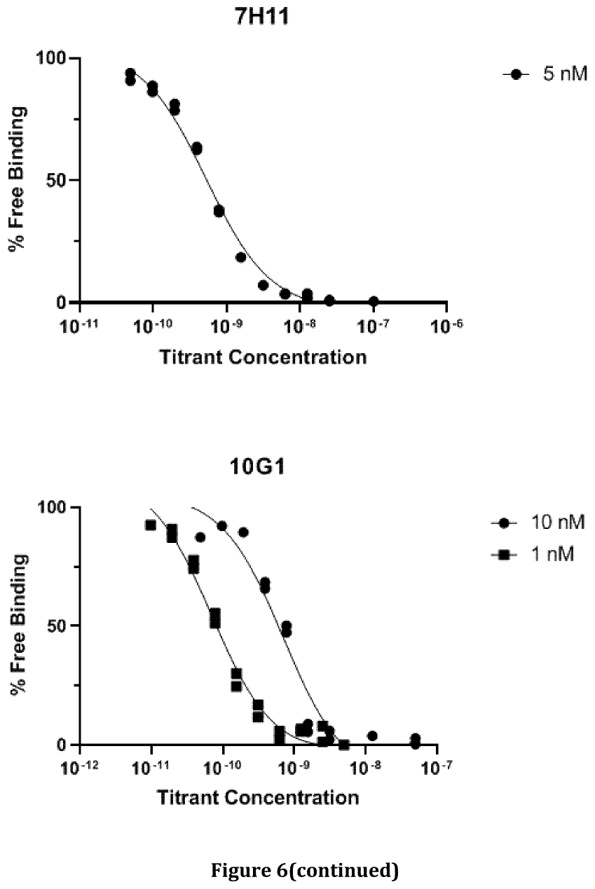

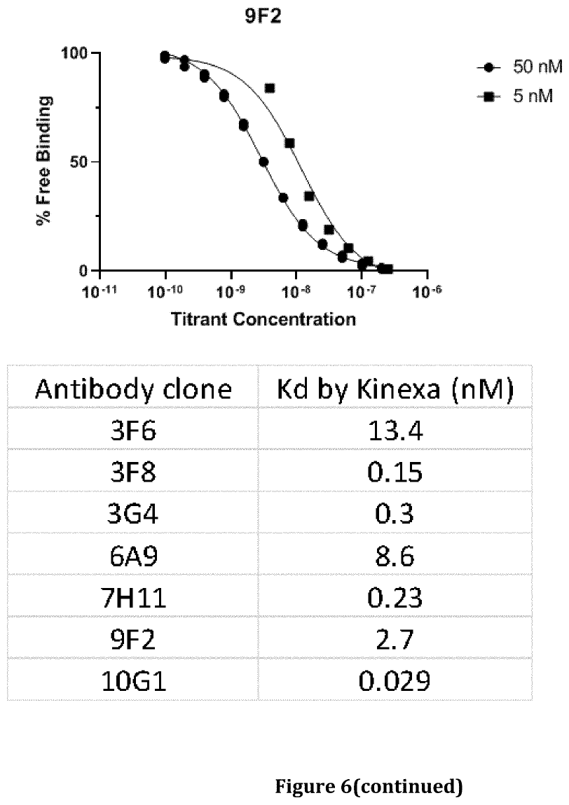

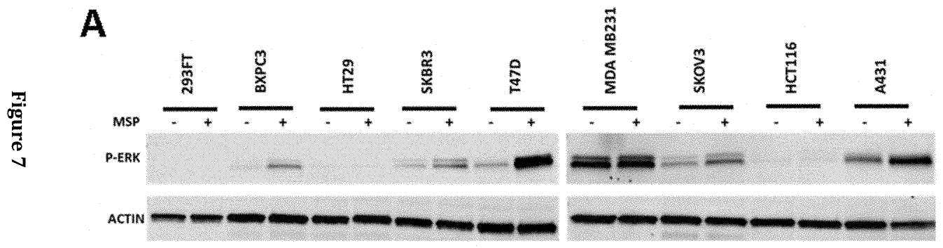

An initial analysis of the binding of the new antibodies using Surface Plasmon Resonance revealed that many of the new antibodies had exceptionally slow off rates making a true determination of their Kds problematic. This limitation of the SPR method for highly avid antibodies has been described previously and the use of kinetic exclusion assay has been proposed as a way to overcome this limitation allowing measurements in the sub nanomolar range. We employed the Kinetic Exclusion assay (KinEXa) method to measure the Kd values of all of the new antibodies using a fixed concentration of the recombinant mammalian expressed protein as the target antigen and a twelve-point dilution curve of each antibody. We dropped antibody 9F6, in this assay as it seemed less avid than the other antibodies in preliminary SPR binding assays. The remaining seven antibodies gave exactly accurate results in duplicate analysis (FIG. 6). The binding data and the resultant Kd values showed a wide range of affinities with four (3F8, 3G4, 7H11 and 10G1) of the seven antibodies binding with sub-nanomolar affinities. The 10G1 antibody stood out with the exceptionally high binding affinity of 29 pM.

### Example 9—Extracellular RON Antibodies Blocks MSP Induced Downstream Signaling Pathways

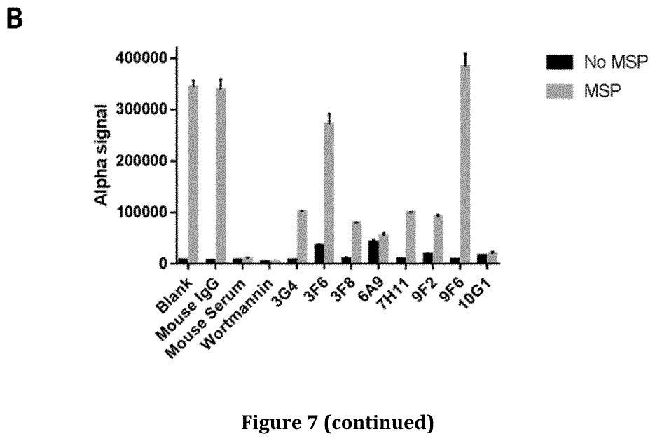

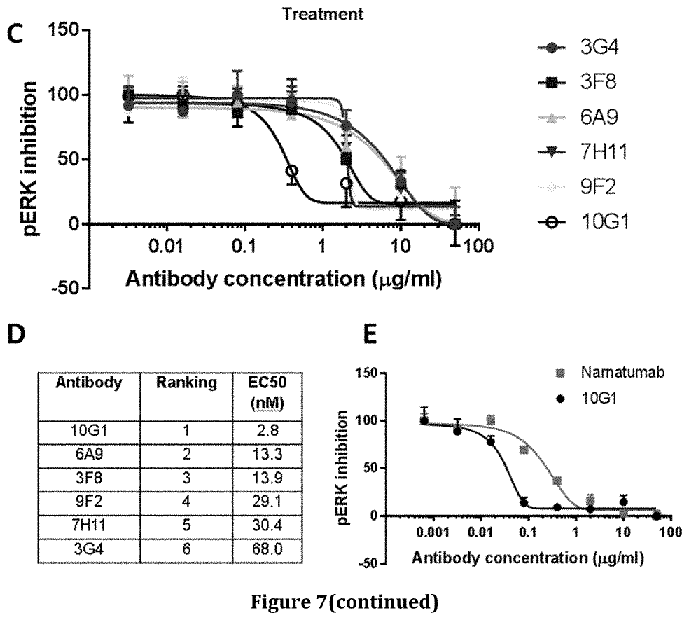

It was investigated whether this panel of antibodies to RON could inhibit the activation of the RON signaling pathway by MSP. First, we analyzed a panel of cell lines and studied the response to MSP by uPsing immunoblotting with an antibody specific to phosphorylated ERK 2 (Thr202/Tyr204). While several of the cell lines responded to MSP stimulation with increased levels of phosphorylated ERK the clearest signal was seen with the T47D cells line. Other cell lines showed weaker responses (HCT116 cells) or had high levels of ligand independent pERK (MDA MB231 cells). To measure the downstream response to MSP accurately in the T47D cells, we used a sensitive alpha screen sure fire assay which provides a quantitative measure of levels of phosphorylated ERK. The assay performed very well with a large dynamic range. We pretreated the cells with antibody (10 μg/ml) an hour before adding MSP for 30 minutes and then processed the samples. Two of the antibodies (3F6 and 9F6) failed to substantially inhibit MSP induced ERK phosphorylation (FIG. 7B) but we were able to use the sure fire assay to titrate the inhibitory activity of the remaining six antibodies (FIG. 7C). The antibodies showed a range of potency from 2.8 nM of 10G1 to 68 nM for 3G4 (FIG. 7D). We used Narnatumab as a control in this assay where it showed an IC50 value of 14.9 nM and is thus a log less potent than 10G1 (FIG. 7E).

In the screening studies, western blot was performed on full length RON transfected 293FT cells or HCT116 cells expressing endogenous RON with our hybridoma supernatants as primary antibodies. However, none of the eight antibodies, unlike 6E6, were able to detect the RON bands on either RON transfected cell lysates or lysates expressing endogenous RON, implying that the antibodies are unable to detect the denatured form of RON produced in the immunoblotting method and detect only folded RON protein in applications like cell staining, ADCC or flow cytometry analyses. (Table 2)

### Example 10—Antibody Dependent Cellular Cytotoxicity as One of the Working Mechanisms of Extracellular RON Antibodies

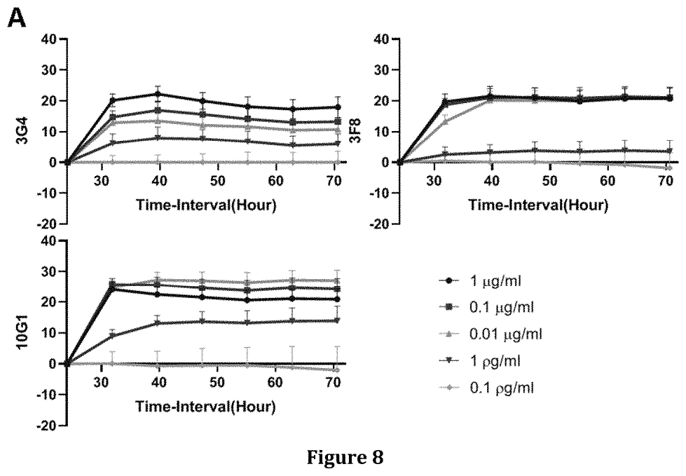

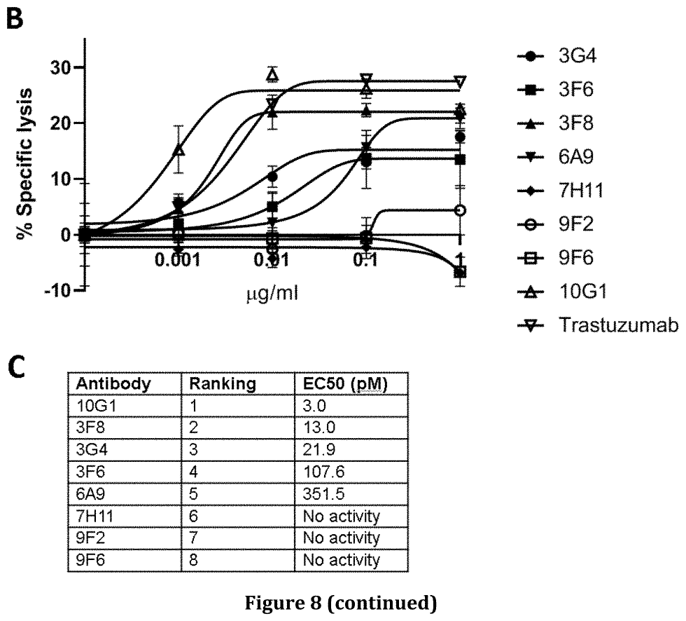

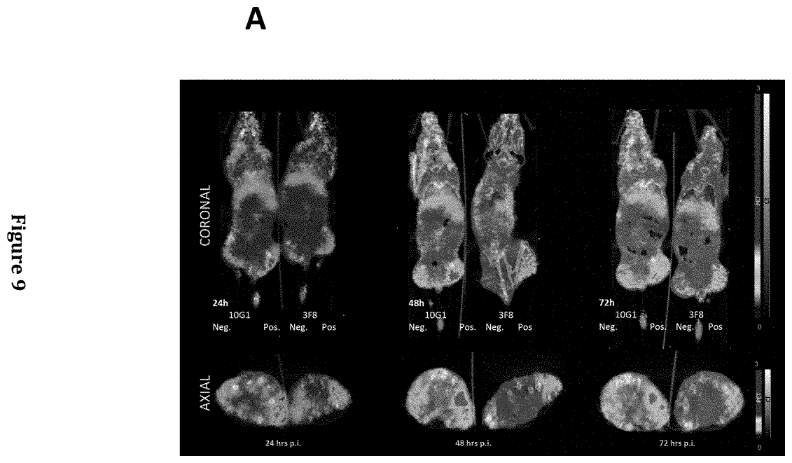

One key mechanism by which therapeutic monoclonal antibodies can exert their effects is through the Fc dependent recruitment of immune effector cells that mediate antibody dependent cell cytotoxicity (ADCC). For this assay we used purified human NK cells and titrated the eight new antibodies over a wide range of concentrations (from 0.1×10⁻¹² g/ml to 1×10⁻⁶ g/ml) using an impedance-based cell cytotoxicity assay (xCELLigence). The assay allows real time monitoring of target cell lysis after the addition of antibodies and effector cells. The plots show (FIG. 8B) that five of the antibodies showed ADCC activity, with dose dependent at cell killing detectable at the first time point (40 minutes) but varied greatly in their potency. Specific lysis data (FIGS. 8A and 8B) were calculated from normalized cell index and baseline (no effector cells) subtracted curves using the xCELLigence RTCA data analysis software. We compared the antibodies to the clinically important anti-HER2 antibody Traztuzumab in this assay and found in keeping with literature that it had an EC₅₀ of around 13 pM. Two of our antibodies (3F8 and 3G4) showed similar values to Traztuzumab while again the 10G1 antibody showed exceptional activity with an EC₅₀ value of 3.0 pM.

### Example 11—Radiolabeled RON Antibodies are Specifically Localized in RON Expressing Tumours

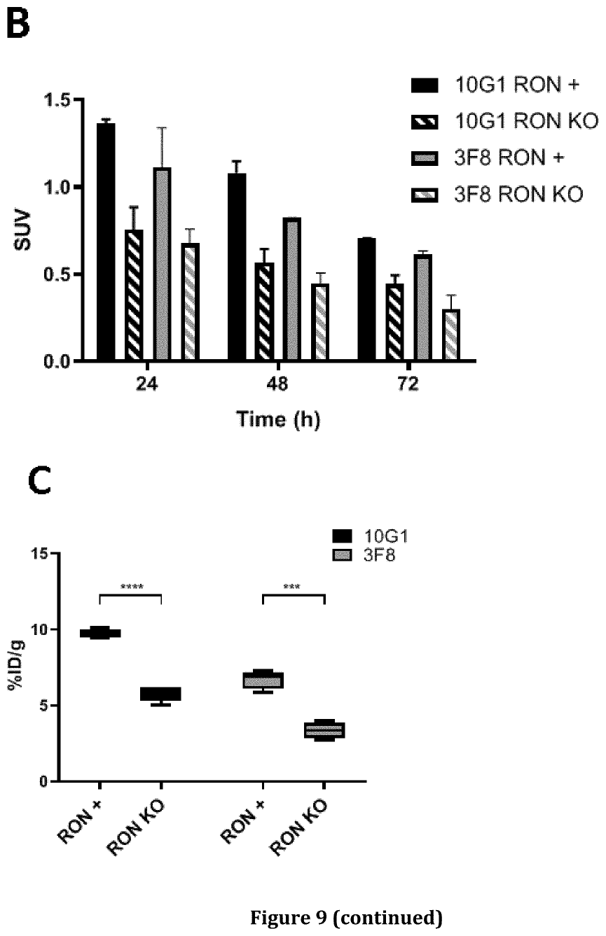

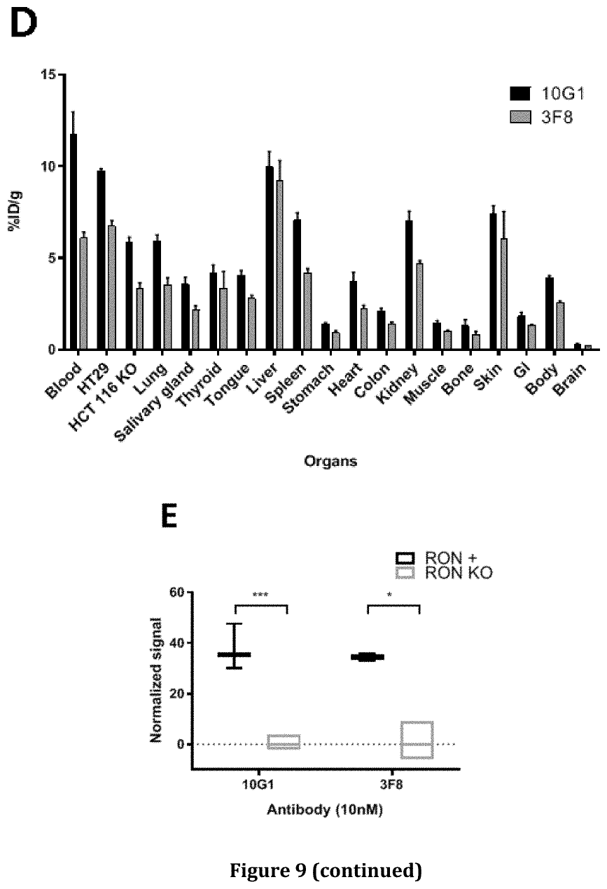

The use of radiolabeled antibodies both for imaging as well as therapeutic radiopharmaceuticals is gaining increased interest. We labeled the 10G1 and 3F8 antibody directed to a surface exposed epitope with ⁸⁹Zr using metal chelate chemistry after DFO-conjugation. Both ⁸⁹Zr-labeled 10G1 and 3F8 antibodies displayed a significantly higher signal on RON positive (HT29) cells compared to RON negative (RON null HCT116) cells in vitro (FIG. 9E).

For the in vivo imaging study, we prepared mice with two xenografts on each flank. The tumour on the right flank was the RON wild type protein expressing cell HT29, while the tumour on the left flank was formed of RON knockout HCT116 cells. PET imaging of the mice at 24, 48 and 72 h post injection of the ⁸⁹Zr-labeled antibodies, as well as biodistribution studies ex vivo at 72 h post injection, showed selective accumulation of the ⁸⁹Zr-labeled antibodies in the RON positive tumors on the right flank (FIG. 5), significantly lower than for the RON-negative tumors on the left flank. Biodistribution studies confirmed the specific uptake in the RON positive tumors, as well as high uptake in the blood and blood-rich organs like liver and spleen, probably due to the long circulation time of the antibodies (FIG. 9D).

Statistical analysis of differences in uptake between antigen-positive and antigen-negative tumors was performed with Graph Pad Prism 8 (GraphPad Software, San Diego, USA), using unpaired student's t-test, with p<0.05 (*), p<0.01 (**), p<0.001 (***) and p<0.0001 (****).

### Methods

### Immunofluorescence Staining of RON Binders in Live Cancer Cells Expressing Endogenous RON

Breast cancer cell line T47D and colorectal cancer cell line HCT116 expressing endogenous RON were seeded in individual wells in a 96 well plate and allowed to adhdksjdf sdlkjsdsdf slkjsdf ere overnight. Cell culture supernatants from hybridoma cells were applied as primary antibodies for an hour following fixation by 4% paraformaldehyde. IgGs were detected with Alexa Fluor 488 conjugated anti-mouse IgG. Cells were counterstained with DAPI and viewed with the Incell Analyzer (GE Healthcare).

### Western Blot Analysis of RON Antibody Clones in Cell Lysates Expressing Endogenous and Transfected RON

Cells were harvested and lysed by sonication in 0.1% Triton X PBS supplemented with protease inhibitor cocktail (Roche). The QuickStart Bradford protein assay (BioRad) was used to determine protein concentration. BSA was used as protein standards. 20 μg of cell lysates were mixed with NuPAGE lithium dodecyl sulphate (LDS) and sample reducing buffer (Thermo Scientific), heated for 5 min at 95° C. and loaded into 4-12% Mini-PROTEAN precast gels (Biorad) for electrophoresis. Separated cell lysates were transferred onto nitrocellulose membranes using the Trans-Blot turbo transfer system device (Biorad). Blocking was performed with 5% milk or bovine serum albumin (BSA) in PBS supplemented with 0.1% tween (TBST). Hybridoma supernatants were applied to individual cell lysate strips as primary antibody and detected with goat anti-mouse IgG (H+L) (Jackson Laboratories). The enhanced chemiluminescence (ECL) reagent used was SuperSignal West Dura Extended Duration Substrate (Thermo Scientific, #34076). Imaging and acquisition were performed with Licor Odyssey Fc and Image Studio (Li-Cor Biosciences).

### Generation of RON Knockout Cell Lines in HCT116, HT29 and T47D by CRISPR Cas9

HCT116, HT29 and T47D cells were chosen for knockout of RON MST1R by CRISPR. A guide RNA containing the spacer (GGCGGGAGGAGCTCCATCG (SEQ ID NO: 152)) that directs Cas9 to cut at the ATG initiation codon of MST1R was cloned into pX458, a plasmid, which contains the gRNA scaffold, spCas9-3×NLS and an EGFP reporter. This plasmid was transfected into HT29 and T47D cells using Lipofectamine 3000, and EGFP-positive cells were selected by FACS. Single clones were isolated and screened for MST1R knockout by directed Sanger sequencing near the Cas9 cut site with the following primers (Forward: ggtccgctatcttggggc (SEQ ID NO: 150); Reverse: ctgggcaccacgtacttcac (SEQ ID NO: 151)).

Flow Cytometry Analysis of RON Antibodies with Fixed and Live Cancer Cells

1×10⁶ cells were harvested from T47D wildtype and T47D RON KO cell lines and washed in PBS. Cells were blocked and either paraformaldehyde fixed or stained live. Primary antibody staining was performed using 10 μg/ml of purified anti-RON antibodies. Cells were subsequently incubated with goat anti-mouse FITC-conjugated secondary antibodies (Invitrogen). FACS analysis was performed using FACS LSRII machine (Becton Dickinson). FlowJo (Tree Star Inc. USA) software was used for data analysis.

### Binding Affinity Studies of RON Antibodies Using Kinetic Exclusion Assay

Mammalian produced recombinant extracellular His-tag RON protein was used for affinity measurements. Affinity determinations were carried out in the fixed antigen format. Antibodies were titrated as two-fold dilutions into a fixed concentration of RON antigen. RON protein was detected using mouse monoclonal to 6×His-Tag (Dylight@650, Thermo Fisher). All affinity measurements were carried out using the KinExa 4000 (Sapidyne Instruments).

### Study of Downstream RON Antibodies Signaling Using Phosphorylation Assays

60000 T47D cells and T47D RON−/− cells were seeded into individual wells in a 96 well plate. Varying concentrations of antibodies, wortmannin or control mouse sera were added to individual wells for an hour before addition of MSP (10 nM) for half an hour. Experiments were ran in triplicates. The cells were harvested for p-ERK level analyses using the AlphaLISA SureFire Ultra p-ERK1/2 (Thr202/Tyr204) Assay Kit (Perkin Elmer) as per manufacturer's protocol.

### Antibody Dependent Cellular Cytotoxicity Assay on RON Antibodies

All antibodies used for the antibody dependent cellular cytotoxicity assay were carried out with engineered chimeric RON antibodies which retained the mouse Fabs with a human Fc backbone and expressed recombinantly. Blood was collected from individuals that provided consent and all protocols were approved by the institutional review board (IRB). NK cells were isolated using the EasySep™ Direct Human NK Isolation Kit (Stemcell Technologies). The effector to target ratio used was 10:1 based on target T47D cells initial seeding. ADCC activities were measured using the xCelligence platform (Roche Applied Science) using plates with detector electrodes that quantify the number of cells attached to the bottom of the wells, reflected by a calculated cell index (CI). The CI was measured every 15 minutes over 72 hours after the antibody treatment. Treatments were performed in triplicates, with averages and standard deviations calculated by the instrument.

### In Vivo Animal Imaging Studies on 3F8 and 10G1

### In Vivo Xenograft Model

Female nu/nu Balb/c mice (n=8) were housed under standard laboratory conditions and fed ad libitum. All experiments complied with Swedish law and were performed with permission from the Uppsala Committee of Animal Research Ethics. Tumor xenografts were formed by subcutaneous inoculation of approximately 1×10⁶ RON-positive HT29 cells on the right posterior leg and 1×10⁶ RON null HCT116 cells on the left posterior leg.

### DFO-Conjugation and ⁸⁹Zr Radiolabeling of 10G1 and 3F8

Conjugation of p-SCN-Bn-Deferoxamine (DFO) to Ab's and ⁸⁹Zr-labelling was performed as described previously (Vosjan, M. J., et al., Nat Protoc, 2010. 5(4): p. 739-43). In brief, Ab's (2 mg/ml dissolved in 0.07 M borax buffer, pH 9.4) were incubated with the bifunctional chelator DFO (B-705, Macrocyclics Dallas, Tex., USA) in the molar ratio of 1:3 (antibody to DFO) for 1 h at 37° C. using a thermomixer at 350 rpm. Unbound-DFO and Ab-DFO were separated with a NAP-5 column equilibrated with 0.25M ammonium acetate (pH 5.4-5.6).

20 MBq ⁸⁹Zr-oxalic (solid target production, clinical grade; kindly provided by Dr. Thuy Tran, KI, Stockholm) acid solution was added to 400 μg Ab at pH 6.8-7.2 (0.1 M Na₂CO₃ and 0.5 M HEPES were added for pH adjustment) and incubated for 1 h at room temperature while gently shaking at 350 rpm. Radiolabeling efficiency and radionuclidic purity (typically >96%) was determined by chromatography strips (ITLC) using 0.2 M citric acid (pH 4.9-5.1) as mobile phase and analysis using a Fujifilm Bas-180011 phosphorimager (Fuji, Tokyo, Japan).

### In Vitro Radioimmunoassay

1 nM of ⁸⁹Zr-10G1 or ⁸⁹Zr-3F8 was added to approximately 0.5*10⁶ HT29 (RON+) or HCT116 (RON KO) cells, and incubated at 37° C., 5% CO₂. After 24 h, cells were washed, trypsinized and counted. Cell-associated radioactivity was measured in a gamma counter (1480 Wizard 3″, Wallace, Turku, Finland). Radioactivity count was adjusted for cell number, and the signal on HT29 cells was normalized to HCT116 signal using GraphPad Prism 8 (GraphPad Software, San Diego, Calif., USA). Statistical analysis of differences in uptake between antigen-positive and antigen-negative cells was performed using Graph Pad Prism 8, using unpaired student's t-test, with p<0.05 (*), p<0.01 (**), and p<0.001 (***).

PET imaging of ⁸⁹Zr-10G1 and ⁸⁹Zr-3F8

Xenografted mice were injected via the tail vein with 50 μg of ⁸⁹Zr-10G1 (n=4) or ⁸⁹Zr-3F8 (n=4) antibodies (injected activity 1.1 and 0.8 MBq), respectively. Whole-body PET/MRI/CT studies were performed under general anesthesia (sevoflurane 2.0-3.5% in 50%/50% medical oxygen+air at 60 ml/min) after 24 h, 48 h and 72 p.i. (i.v.) for ⁸⁹Zr-10G1 (n=2) and ⁸9 Zr-3F8 (n=2). Pre-injected mice were placed under sedation in the gantry of a small-animal nanoScan PET/MR scanner (Mediso Medical Imaging Systems Ltd., Hungary) and a whole-body PET scan was performed for 60 min in list mode followed by a CT scan in nanoScan SPECT/CT scanner (Mediso Medical Imaging Systems Ltd., Hungary) for 5 min. The breathing rate was monitored and animals were placed on the heated bed to prevent hypothermia. PET data was reconstructed into a static image and corrected for the time of injection using the Tera-Tomo™ 3 D reconstruction (6 subsets and 4 iterations). The raw CT data was reconstructed using filtered back projection. PET and CT Dicom files were analyzed with PMOD v3.510 (PMOD Technologies Ltd, Zurich, Switzerland).

Biodistribution of ⁸⁹Zr-10G1 and ⁸⁹Zr-3F8

Biodistribution of RON antibodies in xenografts was studied 72 hours p.i. following PET analyses for ⁸⁹Zr-10G1 (n=4) and ⁸⁹Zr-3F8 (n=4) Animals were euthanized with a mixture of ketamine and xylazine followed by heart puncture. Blood was collected, HCT116 tumors, thyroid (en bloc with larynx), heart, liver, kidneys, spleen, urinary bladder, colon, upper gastrointestinal tract, skin, bone and muscle were excised, weighed and measured in a gamma well-counter (1480 Wizard; Wallace Oy, Turku, Finland). Injection standards were measured for each time point. Radioactivity uptake in the organ was calculated as the percentage of injected dose per gram of tissue (% ID/g). Thyroid uptake was calculated as the percentage of injected dose per organ (% ID/organ).

Statistical analysis of differences in uptake between antigen-positive and antigen-negative tumors was performed with Graph Pad Prism 8 (GraphPad Software, San Diego, USA), using unpaired student's t-test, with p<0.05 (*), p<0.01 (**), p<0.001 (***) and p<0.0001 (****).

### DISCUSSION

### A Panel of New Antibodies to RON

While a vast array of recombinant methods has been devised to produce therapeutic antibodies, these are generally complex and expensive. The use of huge phage libraries has allowed the production of clinically remarkably successful antibodies such as Humira while the use of humanized mice has also been successful. More recently B cell cloning using antigen selections has become effective. However, despite these advances the traditional methods of hybridoma production are very well established and can take advantage of the years of experience gained in immunization protocols and screening methods. Indeed, many of the best-selling therapeutic antibodies have been developed this way, using recombinant methods to humanize the initial mouse antibodies and improve their biophysical properties. Most recently the use of antibody drug conjugates, bi-specific antibodies, and recycling antibodies along with the development of the CAR T cell method has enormously enhanced the potential of developing tumour specific immunotherapies. Receptor tyrosine kinases overexpressed and mutated in cancer cells have proven to be exceptionally good targets for such approaches and antibodies to the EGFR and HER2 receptors have proved very useful in the treatment of breast cancer and colon cancer. However, to date no successful clinical antibodies to the related Met and RON receptors have been produced. The reasons for failure can be many and the tested molecules were only tried as simple antibodies and not as drug conjugates or in recycling configurations for example so that efficacy depends on blocking signaling function or recruiting complement components or NK cells and macrophages through Fc interactions. Here we generate a panel of new antibodies to RON and show superior binding and biological activity to the previous clinical candidate Narnatumab. In particular, the 10G1 antibody binds with picomolar affinity (Kd) and is single figure picomolar active in ADCC assays. 10G1 also performs exceptionally well as an in vivo imaging agent and is a suitable candidate for further development and antibody engineering.

### RON Antibodies Display Profiles Suitable for Clinical Development as Cancer Therapeutics

The cost of bringing an antibody molecule from ‘bench to bedside’ is remarkable. A study done from collecting data from 13 big pharmaceuticals estimated the cost of development of one new biologic molecule to be around $1.8 billion USD. As such, it is critical that most effort should be put into the selection of lead antibody candidates at the early discovery stage, to ensure the highest success rates for the antibody to reach the clinics. Despite the initial promising biological functions shown by RON targeting antibody Narnatumab in preclinical models, the failure of the molecule when tested in phase I clinical trials was partially attributed to the molecule's poor biophysical properties. Lead monoclonal antibody candidates for drug development are typically selected for their high affinities and specificities to the target, potency and biological activities, and abilities to evoke Fc receptor functions. From our panel of new anti-RON antibodies, our antibody with the highest binding affinity of 29 pM 10G1 was chosen as the lead candidate for its ability to specifically bind to and immunoprecipitated RON expressed on the surface of cancer cells, potently block MSP stimulated downstream signaling of RON receptor, and was able to elicit strong antibody dependent cellular cytotoxicity (ADCC) responses which can aid in tumour cell elimination. Moreover, the antibody can be recombinantly expressed and purified at a higher yield than Narnatumab, and is stable and soluble at high concentrations, without the biophysical problems of Narnatumab.

Zirconium Radiolabeled-10G1 can be Developed into Companion Diagnostics for Clinical Imaging of RON Positive Tumours

The use of radiolabeled antibodies for the non-invasive diagnostic and detection of tumours biomarkers on primary tumours and metastases across different tumour types has been long established since the 1980s. Our preliminary study using ⁸⁹Zr-10G1 showed that the antibody was able to specifically colocalize in the xenograft tumour expressing RON but with little cross reactivity to the RON knockout tumour, excellent uptake in the RON-positive tumour and demonstrated a slower excretion from blood compared to 3F8, the other antibody tested in the in vivo assay. Even though our study showed promising data with 10G1 as in a whole IgG format, it will be logical for us to explore the use of 10G1 as an immunoPET agent as smaller antibody fragments like a minibody, ScFv or Fab-fragment, specifically for a few reasons. Firstly, murine based antibodies have a risk of inducing human anti-mouse antibody (HAMA) activity in human patients, using a smaller antibody fragment will reduce the likelihood of this incident. Also, antibodies that are smaller in size may have better tissue penetration, ensuring that the antibodies are taken up into the tumours rather than pool in the blood and hence, gives better contrast immunoPET images. Thirdly, the antibodies will have a shorter circulation time in blood, allowing for a higher tumour to blood ratio and a shorter duration for maximum contrast imaging. As clinical research in oncology moves towards precision medicine, the development of immunoPET agents like 10G1 as companion diagnostics is critical to aid in the achievement of this goal, allowing for an improvement in the method for diagnosing RON related malignancies in the clinic.

1. An antigen specific binding domain which binds to RON (Macrophage stimulating protein receptor or Recepteur d'Origine Nantais), comprising a light chain variable region (VL) and a heavy chain variable region (VH), wherein the heavy chain variable region (VH) comprises Complementarity Determining Regions (CDRs) CDRH1, CDRH2, CDRH3, and the light chain variable region comprises CDRs CDRL1, CDRL2, and CDRL3; wherein:
i. CDRH1 is selected from the group consisting of SEQ ID NO: 140, 10, 28, 46, 67, 83, 107, 124, and a CDRH1 differing from any one of the same in that 1 or 2 amino acids are replaced, deleted or added;
ii. CDRH2 is selected from the group consisting of SEQ ID NO: 141, 11, 29, 47, 68, 84, 108, 125, and a CDRH2 differing from any one of the same in that 1 or 2 amino acids are replaced, deleted or added;
iii. CDRH3 is selected from the group consisting of SEQ ID NO: 142, 12, 30, 48, 69, 85, 109, 126, and a CDRH3 differing from any one of the same in that 1 or 2 amino acids are replaced, deleted or added;
iv. CDRL1 is selected from the group consisting of SEQ ID NO: 132, 1, 19, 37, 60, 75, 98, 115, and a CDRL1 differing from any one of the same in that 1 or 2 amino acids are replaced, deleted or added;
v. CDRL2 is selected from the group consisting of SEQ ID NO: 133, 2, 20, 38, 76, 99, 116, and a CDRL2 differing from any one of the same in that 1 or 2 amino acids are replaced, deleted or added; and
vi. CDRL3 is selected from the group consisting of SEQ ID NO: 134, 3, 21, 39, 61, 77, 100, 117, and a CDRL3 differing from any one of the same in that 1 or 2 amino acids are replaced, deleted or added.

2. The antigen specific binding domain according to claim 1, wherein the CDRs of the light chain variable region are selected from the group consisting of
(a) the CDRL1 is SEQ ID NO: 132, the CDRL2 is SEQ ID NO: 133, the CDRL3 is SEQ ID NO: 134;
(b) the CDRL1 is SEQ ID NO: 1, the CDRL2 is SEQ ID NO: 2, the CDRL3 is SEQ ID NO: 3;
(c) the CDRL1 is SEQ ID NO: 19, the CDRL2 is SEQ ID NO: 20, the CDRL3 is SEQ ID NO: 21;
(d) the CDRL1 is SEQ ID NO: 37, the CDRL2 is SEQ ID NO: 38, the CDRL3 is SEQ ID NO: 39;
(e) the CDRL1 is SEQ ID NO: 60, the CDRL2 is SEQ ID NO: 20, the CDRL3 is SEQ ID NO: 61;
(f) the CDRL1 is SEQ ID NO: 75, the CDRL2 is SEQ ID NO: 76, the CDRL3 is SEQ ID NO: 77;
(g) the CDRL1 is SEQ ID NO: 98, the CDRL2 is SEQ ID NO: 99, the CDRL3 is SEQ ID NO: 100; and
(h) the CDRL1 is SEQ ID NO: 115, the CDRL2 is SEQ ID NO: 116, the CDRL3 is SEQ ID NO: 117.

3. The antigen specific binding domain according to claim 1, wherein the CDRs of the heavy chain variable region are selected from the group consisting of
(a) the CDRH1 is SEQ ID NO: 140, the CRDH2 is SEQ ID NO: 141, the CRDH3 is SEQ ID NO: 142;
(b) the CDRH1 is SEQ ID NO: 10, the CRDH2 is SEQ ID NO: 11, the CRDH3 is SEQ ID NO: 12;
(c) the CDRH1 is SEQ ID NO: 28, the CRDH2 is SEQ ID NO: 29, the CRDH3 is SEQ ID NO: 30;
(d) the CDRH1 is SEQ ID NO: 46, the CRDH2 is SEQ ID NO: 47, the CRDH3 is SEQ ID NO: 48;
(e) the CDRH1 is SEQ ID NO: 67, the CRDH2 is SEQ ID NO: 68, the CRDH3 is SEQ ID NO: 69;
(f) the CDRH1 is SEQ ID NO: 83, the CRDH2 is SEQ ID NO: 84, the CRDH3 is SEQ ID NO: 85;
(g) the CDRH1 is SEQ ID NO: 107, the CRDH2 is SEQ ID NO: 108, the CRDH3 is SEQ ID NO: 109;
(h) the CDRH1 is SEQ ID NO: 124, the CRDH2 is SEQ ID NO: 125, the CRDH3 is SEQ ID NO: 126.

4. The antigen specific binding domain according to claim 1, wherein the CDRs are selected from the group consisting of
(a) the CDRL1 is SEQ ID NO: 132, the CDRL2 is SEQ ID NO: 133, the CDRL3 is SEQ ID NO: 134, the CDRH1 is SEQ ID NO: 140, the CRDH2 is SEQ ID NO: 141, and the CRDH3 is SEQ ID NO: 142;
(b) the CDRL1 is SEQ ID NO: 1, the CDRL2 is SEQ ID NO: 2, the CDRL3 is SEQ ID NO: 3, the CDRH1 is SEQ ID NO: 10, the CRDH2 is SEQ ID NO: 11, and the CRDH3 is SEQ ID NO: 12;
(c) the CDRL1 is SEQ ID NO: 19, the CDRL2 is SEQ ID NO: 20, the CDRL3 is SEQ ID NO: 21 the CDRH1 is SEQ ID NO: 28, the CRDH2 is SEQ ID NO: 29, the CRDH3 is SEQ ID NO: 30;
(d) the CDRL1 is SEQ ID NO: 37, the CDRL2 is SEQ ID NO: 38, the CDRL3 is SEQ ID NO: 39, the CDRH1 is SEQ ID NO: 46, the CRDH2 is SEQ ID NO: 47, and the CRDH3 is SEQ ID NO: 48;
(e) the CDRL1 is SEQ ID NO: 60, the CDRL2 is SEQ ID NO: 20, the CDRL3 is SEQ ID NO: 61, the CDRH1 is SEQ ID NO: 67, the CRDH2 is SEQ ID NO: 68, and the CRDH3 is SEQ ID NO: 69;
(f) the CDRL1 is SEQ ID NO: 75, the CDRL2 is SEQ ID NO: 76, the CDRL3 is SEQ ID NO: 77, the CDRH1 is SEQ ID NO: 83, the CRDH2 is SEQ ID NO: 84, and the CRDH3 is SEQ ID NO: 85;
(g) the CDRL1 is SEQ ID NO: 98, the CDRL2 is SEQ ID NO: 99, the CDRL3 is SEQ ID NO: 100, the CDRH1 is SEQ ID NO: 107, the CRDH2 is SEQ ID NO: 108, and the CRDH3 is SEQ ID NO: 109;
(h) the CDRL1 is SEQ ID NO: 115, the CDRL2 is SEQ ID NO: 116, the CDRL3 is SEQ ID NO: 117, the CDRH1 is SEQ ID NO: 124, the CRDH2 is SEQ ID NO: 125, and the CRDH3 is SEQ ID NO: 126.

5. The antigen specific binding domain according to claim 1, wherein the heavy chain variable region comprises a sequence selected from the group consisting of
(i) SEQ ID NO: 146;
(ii) SEQ ID NO: 130;
(iii) SEQ ID NO: 113;
(iv) SEQ ID NO: 96;
(v) SEQ ID NO: 90;
(vi) SEQ ID NO: 73;
(vii) SEQ ID NO: 58;
(viii) SEQ ID NO: 53;
(ix) SEQ ID NO: 35; and
(x) SEQ ID NO: 17.

6. The antigen specific binding domain according to claim 1, wherein the light chain variable region comprises a sequence selected from the group consisting of
(i) SEQ ID NO: 138;
(ii) SEQ ID NO: 122;
(iii) SEQ ID NO: 105
(iv) SEQ ID NO: 93;
(v) SEQ ID NO: 81
(vi) SEQ ID NO: 65;
(vii) SEQ ID NO: 8;
(viii) SEQ ID NO: 44;
(ix) SEQ ID NO: 26; and
(x) SEQ ID NO: 8.

7. The antigen specific binding domain according to claim 1, comprising a VH/VL pair selected from the group consisting of:
(i) SEQ ID NO: 146 and SEQ ID NO: 138;
(ii) SEQ ID NO: 130 and SEQ ID NO: 122;
(iii) SEQ ID NO: 113 and SEQ ID NO: 105;
(iv) SEQ ID NO: 96 and SEQ ID NO: 93;
(v) SEQ ID NO: 90 and SEQ ID NO: 81;
(vi) SEQ ID NO: 73 and SEQ ID NO: 65;
(vii) SEQ ID NO: 58 and SEQ ID NO: 8;
(viii) SEQ ID NO: 53 and SEQ ID NO: 44
(ix) SEQ ID NO: 35 and SEQ ID NO: 26; and
(x) SEQ ID NO: 17 and SEQ ID NO: 8.

8.-9. (canceled)

10. An antibody molecule comprising the antigen specific binding domain according to claim 1.

11. The antibody molecule according to claim 10, wherein the antibody molecule is a full-length antibody or an antigen binding fragment; or wherein the antibody molecule is a multispecific antibody, optionally a bispecific antibody.

12. (canceled)

13. The antibody molecule according to claim 11, wherein the antibody molecule comprises a first antigen binding domain specifically binding to a first RON (Macrophage stimulating protein receptor or Recepteur d'Origine Nantais) epitope, and a second antigen binding domain specifically binding to a second RON epitope which is different from the first RON epitope.

14. The antibody molecule according to claim 13, wherein each of the first and second antigen binding domains is an antigen specific binding domain, wherein the first and second antigen binding domains are different, and the antigen specific binding domain binds to RON (Macrophage stimulating protein receptor or Recepteur d'Origine Nantais), comprises a light chain variable region (VL) and a heavy chain variable region (VH), wherein the heavy chain variable region (VH) comprises Complementarity Determining Regions (CDRs) CDRH1, CDRH2, CDRH3, and the light chain variable region comprises CDRs CDRL1, CDRL2, and CDRL3; wherein:
i. CDRH1 is selected from the group consisting of SEQ ID NO: 140, 10, 28, 46, 67, 83, 107, 124, and a CDRH1 differing from any one of the same in that 1 or 2 amino acids are replaced, deleted or added;
ii. CDRH2 is selected from the group consisting of SEQ ID NO: 141, 11, 29, 47, 68, 84, 108, 125, and a CDRH2 differing from any one of the same in that 1 or 2 amino acids are replaced, deleted or added;
iii. CDRH3 is selected from the group consisting of SEQ ID NO: 142, 12, 30, 48, 69, 85, 109, 126, and a CDRH3 differing from any one of the same in that 1 or 2 amino acids are replaced, deleted or added;
iv. CDRL1 is selected from the group consisting of SEQ ID NO: 132, 1, 19, 37, 60, 75, 98, 115, and a CDRL1 differing from any one of the same in that 1 or 2 amino acids are replaced, deleted or added;
v. CDRL2 is selected from the group consisting of SEQ ID NO: 133, 2, 20, 38, 76, 99, 116, and a CDRL2 differing from any one of the same in that 1 or 2 amino acids are replaced, deleted or added; and
vi. CDRL3 is selected from the group consisting of SEQ ID NO: 134, 3, 21, 39, 61, 77, 100, 117, and a CDRL3 differing from any one of the same in that 1 or 2 amino acids are replaced, deleted or added.

15. The antibody molecule according to claim 11, wherein the antibody binding fragment is selected from a Fab, a modified Fab, a Fab′, a modified Fab′, a F(ab′)2, an Fv, ds-FV, Fab-Fv, Fab-dsFv, a single domain antibody (e.g. VH or VL or VHH), a scFv, a ds-scFv, and a minibody.

16. The antibody molecule according to claim 10, wherein the antibody molecule binds with RON with a Kd of 15 nM or less, a Kd of 10 nM or less, a Kd of 5 nM or less, a Kd of 1 nM or less, a Kd of 0.6 nM or less, a Kd of 0.3 nM or less, a Kd of 0.15 nM or less, a Kd of 0.1 nM or less, a Kd of 0.03 nM or less, or a Kd of 0.029 nM or less, or a Kd of 0.02 nM or less, or a Kd of 0.01 nM or less, optionally wherein the Kd is determined using a kinetic exclusion assay.

17. The antibody molecule according to claim 10, wherein the antibody molecule inhibits macrophage stimulating protein (MSP) induced pERK activation with an IC₅₀ of 100 nM or less, or with an IC₅₀ of 70 nM or less, or with an IC₅₀ of 31 nM or less, or with an IC₅₀ of 20 nM or less, or with an IC₅₀ of 14.8 nM or less, or with an IC₅₀ of 13.9 nM or less, or with an IC₅₀ of 13.3 nM or less, or with an IC₅₀ of 8 nM or less, or with an IC₅₀ of 5 nM or less, or with an IC₅₀ of 3 nM or less, or with an IC₅₀ of 2.8 nM or less.

18. The antibody molecule according to claim 10, wherein the antibody molecule exhibits antibody dependent cellular cytotoxicity against cancer cells.

19. The antibody molecule according to claim 10, wherein the antibody molecule is a chimeric antibody or a humanized antibody.

20. The antibody molecule according to claim 10, wherein the antibody molecule is conjugated to a payload.

21. The antibody molecule according to claim 20, wherein the payload is selected from any one of a toxin, a polymer, biologically active proteins, nucleic acids and fragments thereof radionuclides chelated metals, nanoparticles or reporter groups.

22.-25. (canceled)

26. A method of treating a patient for cancer comprising administering a therapeutically effective amount of the antigen binding domain according to claim 1.

27. The method according to claim 26, wherein the cancer is RON and C-MET positive.

28.-34. (canceled)
